SEQUENCE LISTING (821 sequences)

SEQ ID NO: 1 - DNA, 90 bp, Escherichia coli
  source 1..90: mol_type = genomic DNA
ttcagccaaaaaacttaagaccgccggtcttgtccactaccttgcagtaatgcggtggacaggatcggcggttttcttttctcttctcaa

SEQ ID NO: 2 - DNA, 57 bp, Escherichia coli
  source 1..57: mol_type = genomic DNA
ggaaacacagaaaaaagcccgcacctgacagtgcgggctttttttttcgaccaaagg

SEQ ID NO: 3 - DNA, 52 bp, Escherichia coli
  source 1..52: mol_type = genomic DNA
gacgaacaataaggcctcccaaatcggggggccttttttattgataacaaaa

SEQ ID NO: 4 - DNA, 57 bp, Escherichia coli
  source 1..57: mol_type = genomic DNA
ctcggtaccaaattccagaaaagagacgctgaaaagcgtcttttttcgttttggtcc

SEQ ID NO: 5 - DNA, 53 bp, Escherichia coli
  source 1..53: mol_type = genomic DNA
aacgcatgagaaagcccccggaagatcaccttccgggggcttttttattgcgc

SEQ ID NO: 6 - DNA, 54 bp, Escherichia coli
  source 1..54: mol_type = genomic DNA
gtcagtttcacctgttttacgtaaaaacccgcttcggcgggtttttacttttgg

SEQ ID NO: 7 - DNA, 47 bp, Escherichia coli
  source 1..47: mol_type = genomic DNA
ccaattattgaacaccctaacgggtgtttttttgtttctggtctccc

SEQ ID NO: 8 - DNA, 49 bp, Escherichia coli
  source 1..49: mol_type = genomic DNA
tccggcaattaaaaaagcggctaaccacgccgctttttttacgtctgca

SEQ ID NO: 9 - DNA, 41 bp, Escherichia coli
  source 1..41: mol_type = genomic DNA
cagataaaaaaaatccttagctttcgctaaggatgatttct

SEQ ID NO: 10 - DNA, 60 bp, Escherichia coli
  source 1..60: mol_type = genomic DNA
gttatgagtcaggaaaaaaggcgacagagtaatctgtcgccttttttctttgcttgcttt

SEQ ID NO: 11 - DNA, 55 bp, Escherichia coli
  source 1..55: mol_type = genomic DNA
taaggttgaaaaataaaaacggcgctaaaaagcgccgttttttttgacggtggta

SEQ ID NO: 12 - DNA, 47 bp, Escherichia coli
  source 1..47: mol_type = genomic DNA
acaattttcgaaaaaacccgcttcggcgggtttttttatagctaaaa

SEQ ID NO: 13 - DNA, 35 bp, Escherichia coli
  source 1..35: mol_type = genomic DNA
taacgtaaaaacccgcttcggcgggtttttttatg

SEQ ID NO: 14 - DNA, 80 bp, Escherichia coli
  source 1..80: mol_type = genomic DNA
ccaggcatcaaataaaacgaaaggctcagtcgaaagactgggcctttcgttttatctgttgtttgtcggtgaacgctctc

SEQ ID NO: 15 - DNA, 44 bp, Escherichia coli
  source 1..44: mol_type = genomic DNA
gatctaactaaaaaggccgctctgcggccttttttcttttcact

SEQ ID NO: 16 - DNA, 47 bp, Escherichia coli
  source 1..47: mol_type = genomic DNA
cgcagatagcaaaaaagcgcctttagggcgcttttttacattggtgg

SEQ ID NO: 17 - DNA, 42 bp, Escherichia coli
  source 1..42: mol_type = genomic DNA
gtaacaacggaaaccggccattgcgccggttttttttggcct

SEQ ID NO: 18 - DNA, 45 bp, Escherichia coli
  source 1..45: mol_type = genomic DNA
agttaaccaaaaaggggggattttatctcccctttaatttttcct

SEQ ID NO: 19 - DNA, 37 bp, Escherichia coli
  source 1..37: mol_type = genomic DNA
aaaaaagcccgcacctgacagtgcgggcttttttttt

SEQ ID NO: 20 - DNA, 47 bp, Escherichia coli
  source 1..47: mol_type = genomic DNA
aggcgactgacgaaacctcgctccggcggggttttttgttatctgca

SEQ ID NO: 21 - DNA, 64 bp, Escherichia coli
  source 1..64: mol_type = genomic DNA
caggcatcaaataaaacgaaaggctcagtcgaaagactgggcctttcgttttatctgttgtttg

SEQ ID NO: 22 - DNA, 42 bp, Escherichia coli
  source 1..42: mol_type = genomic DNA
actgatttttaaggcgactgatgagtcgccttttttttgtct

SEQ ID NO: 23 - DNA, 55 bp, Escherichia coli
  source 1..55: mol_type = genomic DNA
cctgttgagtaatagtcaaaagcctccggtcggaggcttttgactttctgcttac

SEQ ID NO: 24 - DNA, 52 bp, Escherichia coli
  source 1..52: mol_type = genomic DNA
gctgatgccagaaagggtcctgaatttcagggccctttttttacatggattg

SEQ ID NO: 25 - DNA, 52 bp, Bacillus subtilis
  source 1..52: mol_type = genomic DNA
gcttgctgaggatcctaaagccccgaattttttataaattcggggctttttt

SEQ ID NO: 26 - DNA, 34 bp, Escherichia coli
  source 1..34: mol_type = genomic DNA
tgtgaaaaagcccgcgcaagcgggtttttttatg

SEQ ID NO: 27 - DNA, 29 bp, Escherichia coli
  source 1..29: mol_type = genomic DNA
agcccctcaatcgaggggcttttttttgc

SEQ ID NO: 28 - DNA, 54 bp, Escherichia coli
  source 1..54: mol_type = genomic DNA
taccaccgtcaaaaaaaacggcgctttttagcgccgtttttatttttcaacctt

SEQ ID NO: 29 - DNA, 52 bp, Escherichia coli
  source 1..52: mol_type = genomic DNA
acatttaataaaaaaagggcggtcgcaagatcgcccttttttacgtatgaca

SEQ ID NO: 30 - DNA, 42 bp, Escherichia coli
  source 1..42: mol_type = genomic DNA
acctgtaaaaaaggcagccatctggctgccttagtctcccca

SEQ ID NO: 31 - DNA, 36 bp, Escherichia coli
  source 1..36: mol_type = genomic DNA
aagacccccgcaccgaaaggtccgggggtttttttt

SEQ ID NO: 32 - DNA, 45 bp, Escherichia coli
  source 1..45: mol_type = genomic DNA
aagaacgagtaaaaggtcggtttaaccggcctttttattttgtga

SEQ ID NO: 33 - DNA, 38 bp, Escherichia coli
  source 1..38: mol_type = genomic DNA
cagtgaaaaatggcgcccatcggcgccatttttttatg

SEQ ID NO: 34 - DNA, 46 bp, Escherichia coli
  source 1..46: mol_type = genomic DNA
tgaaaacgaaggccggagcatgctccggccttttttatctcttaca

SEQ ID NO: 35 - DNA, 47 bp, Escherichia coli
  source 1..47: mol_type = genomic DNA
caacaatgacaagcggtggagatcttctctgccgctttttttttcat

SEQ ID NO: 36 - DNA, 43 bp, Escherichia coli
  source 1..43: mol_type = genomic DNA
aagcacaaaagccagtctggaaacaggctggcttttttttgcg

SEQ ID NO: 37 - DNA, 47 bp, Escherichia coli
  source 1..47: mol_type = genomic DNA
gttataagacaagggagcgataattcatcgctccctttttcgtgctt

SEQ ID NO: 38 - DNA, 43 bp, Escherichia coli
  source 1..43: mol_type = genomic DNA
aaaaggattcgcggctctgctcttcagagctgcttttatgata

SEQ ID NO: 39 - DNA, 48 bp, Escherichia coli
  source 1..48: mol_type = genomic DNA
caaattaagcagaaggccatcctgacggatggcctttttgcgtttcta

SEQ ID NO: 40 - DNA, 50 bp, Escherichia coli
  source 1..50: mol_type = genomic DNA
ccgccttcacaaatgccgccactcaaacagagcggcatttttcttccccg

SEQ ID NO: 41 - DNA, 39 bp, Escherichia coli
  source 1..39: mol_type = genomic DNA
tgcctcaattagcggctcatgtagccgctttttctgcgc

SEQ ID NO: 42 - DNA, 40 bp, Escherichia coli
  source 1..40: mol_type = genomic DNA
ggcagcatgctgccaggtgatccccctggccacctctttt

SEQ ID NO: 43 - DNA, 34 bp, Escherichia coli
  source 1..34: mol_type = genomic DNA
tacgtaaaaacccgcttcggcgggtttttacttt

SEQ ID NO: 44 - DNA, 48 bp, Escherichia coli
  source 1..48: mol_type = genomic DNA
taatcattcttagcgtgaccgggaagtcggtcacgctacctcttctga

SEQ ID NO: 45 - DNA, 49 bp, Escherichia coli
  source 1..49: mol_type = genomic DNA
cttattccataacaaagccgggtaattcccggctttgttgtatctgaac

SEQ ID NO: 46 - DNA, 41 bp, Escherichia coli
  source 1..41: mol_type = genomic DNA
aataaatggatgccctgcgtaagcggggcatttttcttcct

SEQ ID NO: 47 - DNA, 35 bp, Escherichia coli
  source 1..35: mol_type = genomic DNA
atcgaacaaagggcctgcgggcccttttttcattg

SEQ ID NO: 48 - DNA, 55 bp, Escherichia coli
  source 1..55: mol_type = genomic DNA
agaaacagcaaacaatccaaaacgccgcgttcagcggcgttttttctgcttttct

SEQ ID NO: 49 - DNA, 42 bp, Escherichia coli
  source 1..42: mol_type = genomic DNA
cgtgttcctgaacgcccgcatatgcgggcgttttgctttttg

SEQ ID NO: 50 - DNA, 47 bp, Escherichia coli
  source 1..47: mol_type = genomic DNA
tgctcgtaccaggcccctgcaatttcaacaggggcctttttttatcc

SEQ ID NO: 51 - DNA, 38 bp, Escherichia coli
  source 1..38: mol_type = genomic DNA
aattccatcgggtccgaattttcggaccttttctccgc

SEQ ID NO: 52 - DNA, 52 bp, Escherichia coli
  source 1..52: mol_type = genomic DNA
ttcctgatgtaatgccggatgaccttcgtgtcatccggcatttttcttttca

SEQ ID NO: 53 - DNA, 76 bp, Escherichia coli
  source 1..76: mol_type = genomic DNA
atgggaggcgtttcgtcgtgtgaaacagaatgcgaagacgaacaataaaggcctcccaaatcggggggcctttttt

SEQ ID NO: 54 - DNA, 78 bp, Escherichia coli
  source 1..78: mol_type = genomic DNA
tagcgtaaaagcaaaacacaaatctatccatgcaagcattcaccgccggtttactggcggttttttttcgccgtcata

SEQ ID NO: 55 - DNA, 49 bp, Escherichia coli
  source 1..49: mol_type = genomic DNA
gtgaagtaatacaaaacaggcccaggcggcctgttttgtctttttaatg

SEQ ID NO: 56 - DNA, 47 bp, Escherichia coli
  source 1..47: mol_type = genomic DNA
atctcctttcacggcccattcctcatggatgggccgtttatttcccc

SEQ ID NO: 57 - DNA, 36 bp, Escherichia coli
  source 1..36: mol_type = genomic DNA
cccgcacttaacccgcttcggcgggtttttgttttt

SEQ ID NO: 58 - DNA, 45 bp, Escherichia coli
  source 1..45: mol_type = genomic DNA
caaacaatccaaaacgccgcgttcagcggcgttttttctgctttt

SEQ ID NO: 59 - DNA, 40 bp, Escherichia coli
  source 1..40: mol_type = genomic DNA
ctctgcacaaacgccaccttttcggtggcgttttttatcg

SEQ ID NO: 60 - DNA, 40 bp, Escherichia coli
  source 1..40: mol_type = genomic DNA
gatggctccgatggataaccagcgccgcttaagtcaggaa

SEQ ID NO: 61 - DNA, 37 bp, Escherichia coli
  source 1..37: mol_type = genomic DNA
agcgtcaaaaggccggattttccggccttttttatta

SEQ ID NO: 62 - DNA, 51 bp, Escherichia coli
  source 1..51: mol_type = genomic DNA
gcataatgacaaaaaagggcgctttcactagcgccttttttatttacgcgt

SEQ ID NO: 63 - DNA, 44 bp, Escherichia coli
  source 1..44: mol_type = genomic DNA
tctgaatgcgtgcccattcctgacggaatgggcatttctgcgca

SEQ ID NO: 64 - DNA, 41 bp, Escherichia coli
  source 1..41: mol_type = genomic DNA
agaaatcatccttagcgaaagctaaggattttttttatctg

SEQ ID NO: 65 - DNA, 46 bp, Aliivibrio fischeri
  source 1..46: mol_type = genomic DNA
aaataataaaaaagccggattaataatctggctttttatattctct

SEQ ID NO: 66 - DNA, 39 bp, Escherichia coli
  source 1..39: mol_type = genomic DNA
aacgagaaaagccaacctgcgggttggcttttttatgca

SEQ ID NO: 67 - DNA, 47 bp, Escherichia coli
  source 1..47: mol_type = genomic DNA
gtcagtcgtcagacgccggttaatccggcgttttttttgacgcccac

SEQ ID NO: 68 - DNA, 53 bp, Escherichia coli
  source 1..53: mol_type = genomic DNA
ctaatcttttcaacgcctggcactgccgggcgttgttctttttaacttcaggc

SEQ ID NO: 69 - DNA, 48 bp, Escherichia coli
  source 1..48: mol_type = genomic DNA
aagtcgcaccaaaggggagcgggaaaccgctccccttttatatttagc

SEQ ID NO: 70 - DNA, 42 bp, Escherichia coli
  source 1..42: mol_type = genomic DNA
cagaaatcatccttagcgaaagctaaggattttttttatctg

SEQ ID NO: 71 - DNA, 54 bp, Escherichia coli
  source 1..54: mol_type = genomic DNA
tgcttgattaaaaaggcgctactcggcatggggaagcgccttttttataggtgt

SEQ ID NO: 72 - DNA, 54 bp, Escherichia coli
  source 1..54: mol_type = genomic DNA
tgaacaacatcgcgcttatcgttaaggtaagcgcgtattttttttacccgccag

SEQ ID NO: 73 - DNA, 36 bp, Escherichia coli
  source 1..36: mol_type = genomic DNA
ttatataaagccccgatttatcggggttttttgtta

SEQ ID NO: 74 - DNA, 48 bp, Escherichia coli
  source 1..48: mol_type = genomic DNA
atttgtaccgaaaaccccggggcgtgctccggggttttttcttatcaa

SEQ ID NO: 75 - DNA, 41 bp, Escherichia coli
  source 1..41: mol_type = genomic DNA
atcaaataatgcctgatagcacatatcaggcgttgtcctca

SEQ ID NO: 76 - DNA, 38 bp, Escherichia coli
  source 1..38: mol_type = genomic DNA
cattgtttgatggggctgaaaggccccatttttattgg

SEQ ID NO: 77 - DNA, 42 bp, Escherichia coli
  source 1..42: mol_type = genomic DNA
tttaaagagcaaacccctcaaacgaggggttttttgttgttt

SEQ ID NO: 78 - DNA, 41 bp, Escherichia coli
  source 1..41: mol_type = genomic DNA
cctctacctgcttcggccgataaagccgacgataatactcc

SEQ ID NO: 79 - DNA, 40 bp, Escherichia coli
  source 1..40: mol_type = genomic DNA
gccagttgaaagagggagctagtctccctcttttcgtttc

SEQ ID NO: 80 - DNA, 51 bp, Escherichia coli
  source 1..51: mol_type = genomic DNA
gcccggaccaggccgcaggggggaaactctgcggcctttttcgttcttact

SEQ ID NO: 81 - DNA, 80 bp, Escherichia coli
  source 1..80: mol_type = genomic DNA
tgtaattagatttgactgaaatcgtacagtaaaaagcgtacaataaaggctccacgaaagtggggccttttttagcgcga

SEQ ID NO: 82 - DNA, 38 bp, Escherichia coli
  source 1..38: mol_type = genomic DNA
atcaaaaaggagccgcctgagggcggcttctttttgtg

SEQ ID NO: 83 - DNA, 47 bp, Escherichia coli
  source 1..47: mol_type = genomic DNA
cgagtttcagaaaagggggcctgagtggccccttttttcaagctgac

SEQ ID NO: 84 - DNA, 39 bp, Escherichia coli
  source 1..39: mol_type = genomic DNA
tacgaataaacggctcagaaatgagccgtttattttttc

SEQ ID NO: 85 - DNA, 49 bp, Escherichia coli
  source 1..49: mol_type = genomic DNA
acgagccaataaaaataccggcgttatgccggtatttttttacgaaaga

SEQ ID NO: 86 - DNA, 48 bp, Escherichia coli
  source 1..48: mol_type = genomic DNA
aagttatgcgaaaatgccggtcttgttaccggcattttttatggagaa

SEQ ID NO: 87 - DNA, 46 bp, Escherichia coli
  source 1..46: mol_type = genomic DNA
gaaacgaaccgggagcgctgtgaatacagtgctccctttttttatt

SEQ ID NO: 88 - DNA, 48 bp, Escherichia coli
  source 1..48: mol_type = genomic DNA
ctctttgacgggccaatagcgatattggccatttttttagcgcaacat

SEQ ID NO: 89 - DNA, 35 bp, Escherichia coli
  source 1..35: mol_type = genomic DNA
ggctcaaagacccgctgcggcgggtttttttgtct

SEQ ID NO: 90 - DNA, 37 bp, Escherichia coli
  source 1..37: mol_type = genomic DNA
tattttttgggccggcatgatgccggcttttttttat

SEQ ID NO: 91 - DNA, 29 bp, Escherichia fergusonii
  source 1..29: mol_type = genomic DNA
aaaaggggcctgagtggccccttttttca

SEQ ID NO: 92 - DNA, 44 bp, Escherichia coli
  source 1..44: mol_type = genomic DNA
atgcaggaaagcaggctggagctacccagcctgcagtgaaatta

SEQ ID NO: 93 - DNA, 40 bp, Escherichia coli
  source 1..40: mol_type = genomic DNA
gttattaaatagcctgccatctggcaggctttttttatcg

SEQ ID NO: 94 - DNA, 35 bp, Escherichia coli
  source 1..35: mol_type = genomic DNA
aagtcaaaagcctccggtcggaggcttttgacttt

SEQ ID NO: 95 - DNA, 42 bp, Escherichia coli
  source 1..42: mol_type = genomic DNA
agaataaattcaaccgcccgtcagggcggttgtcatatggag

SEQ ID NO: 96 - DNA, 53 bp, Escherichia coli
  source 1..53: mol_type = genomic DNA
caaccatccgaaaccgctctcatccattcgatgagagcggtttttttaattac

SEQ ID NO: 97 - DNA, 29 bp, Escherichia coli
  source 1..29: mol_type = genomic DNA
cggcccgcctatggcgggccgttttgtat

SEQ ID NO: 98 - DNA, 41 bp, Escherichia coli
  source 1..41: mol_type = genomic DNA
gcctttcaaaagtaagcaacgtctgcttactgcccctctac

SEQ ID NO: 99 - DNA, 39 bp, Escherichia coli
  source 1..39: mol_type = genomic DNA
tccctgtcttcccccacatgctgtgggggttttttttat

SEQ ID NO: 100 - DNA, 40 bp, Escherichia coli
  source 1..40: mol_type = genomic DNA
gcactgatataacgggcctgatggcccgttttagtgtttg

SEQ ID NO: 101 - DNA, 56 bp, Escherichia coli
  source 1..56: mol_type = genomic DNA
ggtattcattgcctgatgcgacgcttacgcgtcttatcatgcctacgggaacctga

SEQ ID NO: 102 - DNA, 48 bp, Escherichia coli
  source 1..48: mol_type = genomic DNA
cacagaatctaagatccctgccatttggcggggatttttttatttgtt

SEQ ID NO: 103 - DNA, 59 bp, Escherichia coli
  source 1..59: mol_type = genomic DNA
caagcactgcaaaaaacagccggacggttttcacctccggctatttttttaattgtgat

SEQ ID NO: 104 - DNA, 64 bp, Escherichia coli
  source 1..64: mol_type = genomic DNA
ccccgatttatcggggttttttgttatctgactacagaataactgggctttaggcccttttttt

SEQ ID NO: 105 - DNA, 43 bp, Escherichia coli
  source 1..43: mol_type = genomic DNA
ctcaccctaaccctctccccagaggggcgaggggaccgaccga

SEQ ID NO: 106 - DNA, 51 bp, Escherichia coli
  source 1..51: mol_type = genomic DNA
tgtagttgtaaggccgtgcttccgaaaggaatgcgcggcttattttcgttt

SEQ ID NO: 107 - DNA, 41 bp, Escherichia coli
  source 1..41: mol_type = genomic DNA
gggaagaggatagtctgccgtctccagactaataaaccgtt

SEQ ID NO: 108 - DNA, 42 bp, Escherichia coli
  source 1..42: mol_type = genomic DNA
agtatgcacacgggcagcacgacgctgcccgatttttttgca

SEQ ID NO: 109 - DNA, 47 bp, Escherichia coli
  source 1..47: mol_type = genomic DNA
tagcaacaaaaaagccgactcacttgcagtcggctttctcattttaa

SEQ ID NO: 110 - DNA, 28 bp, Escherichia coli
  source 1..28: mol_type = genomic DNA
agcccgcctaatgagcgggctttttttt

SEQ ID NO: 111 - DNA, 43 bp, Escherichia coli
  source 1..43: mol_type = genomic DNA
gttgccatttgccctccgctgcggcggggggcttttaaccggg

SEQ ID NO: 112 - DNA, 41 bp, Escherichia coli
  source 1..41: mol_type = genomic DNA
aagcagagaaccctggatgagagtccggggtttttgttttt

SEQ ID NO: 113 - DNA, 35 bp, Escherichia coli
  source 1..35: mol_type = genomic DNA
aaagtcaaaagcctccgaccggaggcttttgactt

SEQ ID NO: 114 - DNA, 39 bp, Escherichia coli
  source 1..39: mol_type = genomic DNA
ctatcctcttcccggtcccctatgccgggttttttttat

SEQ ID NO: 115 - DNA, 47 bp, Escherichia coli
  source 1..47: mol_type = genomic DNA
gtattcgcgcaccccggtctagccggggtcattttttagtggctttt

SEQ ID NO: 116 - DNA, 45 bp, Escherichia coli
  source 1..45: mol_type = genomic DNA
tattgattataaagggctttaatttttggcccttttatttttggt

SEQ ID NO: 117 - DNA, 41 bp, Escherichia coli
  source 1..41: mol_type = genomic DNA
cgtctgcgtatggaacgtggtaacggttctactgaagattt

SEQ ID NO: 118 - DNA, 39 bp, Escherichia coli
  source 1..39: mol_type = genomic DNA
tgatcatcaaggcttccttcgggaagcctttctacgtta

SEQ ID NO: 119 - DNA, 48 bp, Escherichia coli
  source 1..48: mol_type = genomic DNA
atgaatgaacaaaaccctctgttactacagagggttttttatcttcaa

SEQ ID NO: 120 - DNA, 42 bp, Escherichia coli
  source 1..42: mol_type = genomic DNA
gtaaaaatacagggctggaatcatccggcccttttttctgat

SEQ ID NO: 121 - DNA, 72 bp, Escherichia coli
  source 1..72: mol_type = genomic DNA
ctaagcgggcaaaacctgaaaaaaattgcttgattcacgtcaggccgtttttttcaggtttttttttggagt

SEQ ID NO: 122 - DNA, 51 bp, Escherichia coli
  source 1..51: mol_type = genomic DNA
gctgttcagatcactggtgcgcgcggagtcgccgccagtgagcaaacgctg

SEQ ID NO: 123 - DNA, 61 bp, Escherichia coli
  source 1..61: mol_type = genomic DNA
gtgaattgtagctgacctgggacttgtacccgggtcggtatttttttgcttctggtcccgg

SEQ ID NO: 124 - DNA, 44 bp, Escherichia coli
  source 1..44: mol_type = genomic DNA
agtttgttcgcccggtagttgtgacgctaccgggttcttttcga

SEQ ID NO: 125 - DNA, 66 bp, Escherichia coli
  source 1..66: mol_type = genomic DNA
ttagtgcccagggttccctctcaccctaaccctctccccggtggggcgaggggactgaccgagcgc

SEQ ID NO: 126 - DNA, 47 bp, Escherichia coli
  source 1..47: mol_type = genomic DNA
gctcataagtaaaaaacggcacctggtgccgtttttttgtctgaaac

SEQ ID NO: 127 - DNA, 43 bp, Escherichia coli
  source 1..43: mol_type = genomic DNA
ccgtttaacacgttctggatgaaatccatatcgcgatagcgca

SEQ ID NO: 128 - DNA, 36 bp, Escherichia coli
  source 1..36: mol_type = genomic DNA
gcgtaaaaaagcacctttttaggtgcttttttgtgg

SEQ ID NO: 129 - DNA, 59 bp, Escherichia coli
  source 1..59: mol_type = genomic DNA
gaaccacctccttagcctgtgtaatctcccttacacgggcttattttttacgcgtaata

SEQ ID NO: 130 - DNA, 40 bp, Escherichia coli
  source 1..40: mol_type = genomic DNA
gaagttcaagggcgcatctactgatgtgccttttttattt

SEQ ID NO: 131 - DNA, 40 bp, Escherichia coli
  source 1..40: mol_type = genomic DNA
actattttctaaaggcgcttcggcgcctttttagtcagat

SEQ ID NO: 132 - DNA, 55 bp, Escherichia coli
  source 1..55: mol_type = genomic DNA
gataaatcagaaacataaaggcgctttcgggtgcctttattatttccagtgaaac

SEQ ID NO: 133 - DNA, 41 bp, Escherichia coli
  source 1..41: mol_type = genomic DNA
ccataaaaaagcgccattcagcgcctttttatcatcccctt

SEQ ID NO: 134 - DNA, 48 bp, Escherichia coli
  source 1..48: mol_type = genomic DNA
ttccgctgaaggcgtaattgtttaaataacattacgccgcctggcctt

SEQ ID NO: 135 - DNA, 38 bp, Escherichia coli
  source 1..38: mol_type = genomic DNA
atgaccttttgcaccgctttgcggtgctttcctggaag

SEQ ID NO: 136 - DNA, 54 bp, Escherichia coli
  source 1..54: mol_type = genomic DNA
taagacgtggcgcatcaggcatcgtgcaccgaatgccggatgcggcgtgaacgc

SEQ ID NO: 137 - DNA, 37 bp, Escherichia coli
  source 1..37: mol_type = genomic DNA
actgttattcggcgctccacggagcgcctttttttct

SEQ ID NO: 138 - DNA, 40 bp, Escherichia coli
  source 1..40: mol_type = genomic DNA
agttttaacgaaggggtggtttcaccccttttgtctttct

SEQ ID NO: 139 - DNA, 48 bp, Escherichia coli
  source 1..48: mol_type = genomic DNA
ccggctcattgcagcgaaataatcctctctttatctgctatacctggt

SEQ ID NO: 140 - DNA, 42 bp, Escherichia coli
  source 1..42: mol_type = genomic DNA
gattttatgaaaaacggcctgcgggccgttttgttttgtctg

SEQ ID NO: 141 - DNA, 41 bp, Escherichia coli
  source 1..41: mol_type = genomic DNA
aaggacaccagagcctgccaatggcaggctcagactgatga

SEQ ID NO: 142 - DNA, 38 bp, Escherichia coli
  source 1..38: mol_type = genomic DNA
agtcaaaagcctccgaccggaggcttttgactattact

SEQ ID NO: 143 - DNA, 41 bp, Escherichia coli
  source 1..41: mol_type = genomic DNA
aaagtcaaaatgcccgatcgaggatcgggcatttttgtagc

SEQ ID NO: 144 - DNA, 51 bp, Escherichia coli
  source 1..51: mol_type = genomic DNA
aagatgaacaaaactaaagcgccacaagggcgctttagtttgttttccggt

SEQ ID NO: 145 - DNA, 45 bp, Escherichia coli
  source 1..45: mol_type = genomic DNA
atctctctacgccctcacccgtacagggtgagggcaataatcttt

SEQ ID NO: 146 - DNA, 26 bp, Escherichia coli
  source 1..26: mol_type = genomic DNA
agcctcccagtggaggctttttttgt

SEQ ID NO: 147 - DNA, 33 bp, Escherichia coli
  source 1..33: mol_type = genomic DNA
aaagcccccggaagatcaccttccgggggcttt

SEQ ID NO: 148 - DNA, 45 bp, Escherichia coli
  source 1..45: mol_type = genomic DNA
cacaatgatgcccggttgcttttcacaaccgggcatttttttaac

SEQ ID NO: 149 - DNA, 35 bp, Escherichia coli
  source 1..35: mol_type = genomic DNA
cctgtcctgatcgtttcctgaacgataaattgtga

SEQ ID NO: 150 - DNA, 47 bp, Escherichia coli
  source 1..47: mol_type = genomic DNA
gcgcgggtaggcctgataagcgaagcgctatcaggcatttttcccta

SEQ ID NO: 151 - DNA, 72 bp, Escherichia coli
  source 1..72: mol_type = genomic DNA
tccggcaaaaaaacgggcaaggtgtcaccaccctgccctttttctttaaaaccgaaaagattacttcgcgtt

SEQ ID NO: 152 - DNA, 45 bp, Escherichia coli
  source 1..45: mol_type = genomic DNA
tacttcttactcgcccatctgcaacggatgggcgaatttataccc

SEQ ID NO: 153 - DNA, 53 bp, Escherichia coli
  source 1..53: mol_type = genomic DNA
gaggtgatttaaattcatccccggcggcaagccggggagatttcattacggca

SEQ ID NO: 154 - DNA, 52 bp, Escherichia coli
  source 1..52: mol_type = genomic DNA
gtaagcagaaagtcaaaagcctccgaccggaggcttttgactattactcaac

SEQ ID NO: 155 - DNA, 36 bp, Escherichia coli
  source 1..36: mol_type = genomic DNA
cagattgctgacaacgtgcgcgttgttcatgccgga

SEQ ID NO: 156 - DNA, 47 bp, Escherichia coli
  source 1..47: mol_type = genomic DNA
ctgattatgaaaatgccgggatttattcccggcatttctgattgtta

SEQ ID NO: 157 - DNA, 46 bp, Escherichia coli
  source 1..46: mol_type = genomic DNA
cagccctaatcagcgttgcaggataaagcaccgctcactcttcaac

SEQ ID NO: 158 - DNA, 98 bp, Escherichia coli
  source 1..98: mol_type = genomic DNA
acggcttcccacgtcagaccaaaacgcgccaggtatttgcgtagccgatccgcgtcattgacgctggctttgccctggcgcgaaacgtcaaaaagctg

SEQ ID NO: 159 - DNA, 60 bp, Escherichia coli
  source 1..60: mol_type = genomic DNA
actgcgttaagttataccgcctcggtcagttccggctgaggcgtttccactccctccgca

SEQ ID NO: 160 - DNA, 39 bp, Escherichia coli
  source 1..39: mol_type = genomic DNA
aactccgctgttgccctgtttcagggcaattttgcaacc

SEQ ID NO: 161 - DNA, 55 bp, Escherichia coli
  source 1..55: mol_type = genomic DNA
agtaagcgcgaatatgcctgatggtgcaacaccatcaggcatattaaattatgct

SEQ ID NO: 162 - DNA, 48 bp, Escherichia coli
  source 1..48: mol_type = genomic DNA
aatcgcgttacggggaggaagttttttcagatactcccggaacgcctg

SEQ ID NO: 163 - DNA, 41 bp, Escherichia coli
  source 1..41: mol_type = genomic DNA
taatcgcctcgccccggtatcgtgccggggctttttcactt

SEQ ID NO: 164 - DNA, 46 bp, Escherichia coli
  source 1..46: mol_type = genomic DNA
ttacccgcaaagtgcgctgctgcttagcagcgcactgcttggtggg

SEQ ID NO: 165 - DNA, 33 bp, synthetic construct
  source 1..33: mol_type = other DNA
aaaatcaaaggatcttcttgagatccttttttt

SEQ ID NO: 166 - DNA, 64 bp, Escherichia coli
  source 1..64: mol_type = genomic DNA
ttaaagcaccttaattatcgtcgcattcagaacagtctggatgcgatgcgttaattctttcttt

SEQ ID NO: 167 - DNA, 36 bp, Escherichia coli
  source 1..36: mol_type = genomic DNA
ctgcccttttccctgctcctggacggttttacccct

SEQ ID NO: 168 - DNA, 168 bp, Escherichia coli
  source 1..168: mol_type = genomic DNA
cccaatgactacttccatgctcaacgcaaaactactaccaactgcgccatccgccgcagtggtcgtcgtgcgtgtggtggtggtcgtcggcaatgcgccgtagggactggaacaacacacgattccaaaaccccgccggcgcaaaccgggcggggtttttcgtttaag

SEQ ID NO: 169 - DNA, 39 bp, Escherichia coli
  source 1..39: mol_type = genomic DNA
ccgtattaccccgcgccgggaatgcgcggccgccaattt

SEQ ID NO: 170 - DNA, 51 bp, Escherichia coli
  source 1..51: mol_type = genomic DNA
taccatgtccttattgaccccgtatattacggggtcgtttttgtgcggaat

SEQ ID NO: 171 - DNA, 39 bp, Escherichia coli
  source 1..39: mol_type = genomic DNA
agtcagaaagccgccgacatgctcggcggtttttctgaa

SEQ ID NO: 172 - DNA, 49 bp, Escherichia coli
  source 1..49: mol_type = genomic DNA
ctgatgaaaaggtgccggatgatgtgaatcatccggcactggattatta

SEQ ID NO: 173 - DNA, 52 bp, Escherichia coli
  source 1..52: mol_type = genomic DNA
taatcggatgcaggcaggggaagtgtctgtttaccctgcctggtctgatacg

SEQ ID NO: 174 - DNA, 46 bp, Escherichia coli
  source 1..46: mol_type = genomic DNA
gtgtaagtttagccggataacgcgccagatccggcttacatctctg

SEQ ID NO: 175 - DNA, 43 bp, Escherichia coli
  source 1..43: mol_type = genomic DNA
atgctttagtaagggcgcgacgtttgcgccctttgtaggccgg

SEQ ID NO: 176 - DNA, 40 bp, Escherichia coli
  source 1..40: mol_type = genomic DNA
aaaaaatcacagggcagggaaacctgcccttgtttcagcg

SEQ ID NO: 177 - DNA, 65 bp, Escherichia coli
  source 1..65: mol_type = genomic DNA
gctccgccgtcacggttgcaggaaagctaagggacttagcctgcggcggttttgtttggcttcag

SEQ ID NO: 178 - DNA, 43 bp, Escherichia coli
  source 1..43: mol_type = genomic DNA
ctgaaatatccagcggatcaagaaaattcgttggatatttttt

SEQ ID NO: 179 - DNA, 49 bp, Escherichia coli
  source 1..49: mol_type = genomic DNA
ctttcttttgcagcagactggcaggagtgcgagtctgctcgcataatca

SEQ ID NO: 180 - DNA, 42 bp, Escherichia coli
  source 1..42: mol_type = genomic DNA
cgattgagccttccagtccttcgggactggaatttttttgtt

SEQ ID NO: 181 - DNA, 47 bp, Escherichia coli
  source 1..47: mol_type = genomic DNA
ccgacgtaaaaagacggtaagtatcgctttcagtcttatgaatatcg

SEQ ID NO: 182 - DNA, 42 bp, Escherichia coli
  source 1..42: mol_type = genomic DNA
gtttctcgcgcaggcgctgaaaatagcgcctgtttttatttc

SEQ ID NO: 183 - DNA, 43 bp, Escherichia coli
  source 1..43: mol_type = genomic DNA
ggctggaaaacctggctcgtatgcgtaagcaggacattatttt

SEQ ID NO: 184 - DNA, 45 bp, Escherichia coli
  source 1..45: mol_type = genomic DNA
gaacttgtaggcctgataagcgcagcgtatcaggcaatttttata

SEQ ID NO: 185 - DNA, 42 bp, Escherichia coli
  source 1..42: mol_type = genomic DNA
tcaaacgaaaccaggctatactcaagcctggttttttgatgg

SEQ ID NO: 186 - DNA, 38 bp, Escherichia coli
  source 1..38: mol_type = genomic DNA
cagtaatgctggcgcgccccctcgcgcctgaaaattac

SEQ ID NO: 187 - DNA, 52 bp, Escherichia coli
  source 1..52: mol_type = genomic DNA
ctgagtgaactcccatgagcatagataactatgtgaatgggatgagcgaagg

SEQ ID NO: 188 - DNA, 47 bp, Escherichia coli
  source 1..47: mol_type = genomic DNA
aataagcaataacggtacgacagctgtgtcgtgccgtttgttttttc

SEQ ID NO: 189 - DNA, 45 bp, Escherichia coli
  source 1..45: mol_type = genomic DNA
aaacacgtaggcctgataagcgaagcgcatcaggcagttttgcgt

SEQ ID NO: 190 - DNA, 51 bp, Escherichia coli
  source 1..51: mol_type = genomic DNA
ggcaataactcttttcgccgagcaggatgctcgcgaaaagaaactgtgatt

SEQ ID NO: 191 - DNA, 51 bp, Escherichia coli
  source 1..51: mol_type = genomic DNA
agcaggaaagagtaaggctgaaccttcatgttcaaccttactctcatttac

SEQ ID NO: 192 - DNA, 50 bp, Escherichia coli
  source 1..50: mol_type = genomic DNA
tgcgttattttcggcaccttttatgtagcgaaggtgccggaatatattct

SEQ ID NO: 193 - DNA, 50 bp, Escherichia coli
  source 1..50: mol_type = genomic DNA
tgatgtaagtaaaagagccggcccaacggccggcttttttctggtaatct

SEQ ID NO: 194 - DNA, 42 bp, Escherichia coli
  source 1..42: mol_type = genomic DNA
caaaataacaaaacccaccttaaggtgggtttcgccagagaa

SEQ ID NO: 195 - DNA, 43 bp, Escherichia coli
  source 1..43: mol_type = genomic DNA
atattctgaaatatccagcggatcaagaaaattcgttggatat

SEQ ID NO: 196 - DNA, 33 bp, Escherichia coli
  source 1..33: mol_type = genomic DNA
ctaagcgttgtccccagtggggatgtgacgaag

SEQ ID NO: 197 - DNA, 45 bp, Escherichia coli
  source 1..45: mol_type = genomic DNA
gttatcggtgcagagcccgggcgaaccgggctttgttttgggtgt

SEQ ID NO: 198 - DNA, 75 bp, Escherichia coli
  source 1..75: mol_type = genomic DNA
cgaataatccgattacggctacgcttctaatgttccccttgaatggagtcgaagaatgcgtaatcccacgctgtt

SEQ ID NO: 199 - DNA, 46 bp, Aliivibrio fischeri
  source 1..46: mol_type = genomic DNA
agagaatataaaaagccagattattaatccggcttttttattattt

SEQ ID NO: 200 - DNA, 37 bp, Escherichia coli
  source 1..37: mol_type = genomic DNA
aatgatctgacgggggaacctcccccgttaaaaaaat

SEQ ID NO: 201 - DNA, 33 bp, Escherichia coli
  source 1..33: mol_type = genomic DNA
cagaataactgggctttaggccctttttttatg

SEQ ID NO: 202 - DNA, 26 bp, Escherichia coli
  source 1..26: mol_type = genomic DNA
gccgccagttccgctggcggcatttt

SEQ ID NO: 203 - DNA, 44 bp, Escherichia coli
  source 1..44: mol_type = genomic DNA
ttaacccctaccccacgcgtacaaccgcgtggggagacgacgcg

SEQ ID NO: 204 - DNA, 49 bp, Escherichia coli
  source 1..49: mol_type = genomic DNA
cttgaaaaataaagtattagcgttctgcgttaagacttttttcatgggt

SEQ ID NO: 205 - DNA, 45 bp, Escherichia coli
  source 1..45: mol_type = genomic DNA
aataatttccgcgtcatgcttcacgccgcagatgcgttggctgcg

SEQ ID NO: 206 - DNA, 39 bp, Escherichia coli
  source 1..39: mol_type = genomic DNA
ttctgtgctgtgccattgccgccagcgcattaatttcca

SEQ ID NO: 207 - DNA, 37 bp, Escherichia coli
  source 1..37: mol_type = genomic DNA
taagaaaagcgagagttacagctctcacttatttgtt

SEQ ID NO: 208 - DNA, 31 bp, Escherichia coli
  source 1..31: mol_type = genomic DNA
ccctcttgataacccaagagggcatttttta

SEQ ID NO: 209 - DNA, 36 bp, Escherichia coli
  source 1..36: mol_type = genomic DNA
ggcgtgagattggaatacaatttcgcgccttttgtt

SEQ ID NO: 210 - DNA, 50 bp, Escherichia coli
  source 1..50: mol_type = genomic DNA
cgaaccgtaggtcggataaggcgctcgcgccgcatccgacaaatgtgttc

SEQ ID NO: 211 - DNA, 48 bp, Escherichia coli
  source 1..48: mol_type = genomic DNA
agtaaaacccatgccggatgcgccagcatccggcataataccgattac

SEQ ID NO: 212 - DNA, 45 bp, Escherichia coli
  source 1..45: mol_type = genomic DNA
ataacagaaaactcccccgcgagaagcgggggagtcgctggttaa

SEQ ID NO: 213 - DNA, 45 bp, Escherichia coli
  source 1..45: mol_type = genomic DNA
aattattggcaaaaggcaaccacaggctgcctttttctttgactc

SEQ ID NO: 214 - DNA, 44 bp, Escherichia coli
  source 1..44: mol_type = genomic DNA
cagccactgctctgaccacaagtaattgttcagattgataaaac

SEQ ID NO: 215 - DNA, 51 bp, Escherichia coli
  source 1..51: mol_type = genomic DNA
ccggctggacaatgttaccgataacagttacccgtaacatttttaattctt

SEQ ID NO: 216 - DNA, 45 bp, Escherichia coli
  source 1..45: mol_type = genomic DNA
atgctaaattgcccgatgcgctgcgcttatcgggccttcatggtt

SEQ ID NO: 217 - DNA, 43 bp, Escherichia coli
  source 1..43: mol_type = genomic DNA
aggaaagtaagtgccggatatgaaatccggcacctgtcagact

SEQ ID NO: 218 - DNA, 53 bp, Escherichia coli
  source 1..53: mol_type = genomic DNA
tttaatatgacaccggactccgttcctcgatggggtccggttgttttattcac

SEQ ID NO: 219 - DNA, 61 bp, Escherichia coli
  source 1..61: mol_type = genomic DNA
aacagtaacttttccggcttcccgttcgtcagtacctcgggaagccgccaaccaggataaa

SEQ ID NO: 220 - DNA, 33 bp, Escherichia coli
  source 1..33: mol_type = genomic DNA
ccctcttgggttatcaagagggtcattatattt

SEQ ID NO: 221 - DNA, 44 bp, Escherichia coli
  source 1..44: mol_type = genomic DNA
taaagaaactcgccggatgaaaagtcatccggcgtcatattact

SEQ ID NO: 222 - DNA, 49 bp, Escherichia coli
  source 1..49: mol_type = genomic DNA
gggtaaaaaaatgcctgactgctttgtgcgatcaggcattctcgaatta

SEQ ID NO: 223 - DNA, 34 bp, Escherichia coli
  source 1..34: mol_type = genomic DNA
ccgattatttacgcaaatttgcgtgccaaaattt

SEQ ID NO: 224 - DNA, 51 bp, Escherichia coli
  source 1..51: mol_type = genomic DNA
aaaactccaggccgggtacggtgttttacgccgcatccggcattacaaaat

SEQ ID NO: 225 - DNA, 41 bp, Escherichia coli
  source 1..41: mol_type = genomic DNA
acgcgtacaaccgcgtggggagacgacgcggatttttaact

SEQ ID NO: 226 - DNA, 48 bp, Escherichia coli
  source 1..48: mol_type = genomic DNA
gtttaacggctctggcggagctcccaggctccgccagatttatttact

SEQ ID NO: 227 - DNA, 47 bp, Escherichia coli
  source 1..47: mol_type = genomic DNA
agaaattgagattcatccactacttgcatggatgagtaatgattaat

SEQ ID NO: 228 - DNA, 71 bp, Escherichia coli
  source 1..71: mol_type = genomic DNA
taaagtatgtatccccaaaataattcgagtcattgcatctgtggctagaagtatgaagggattaaccataa

SEQ ID NO: 229 - DNA, 41 bp, Escherichia coli
  source 1..41: mol_type = genomic DNA
acaagaaaaaaggcacgtcatctgacgtgccttttttattt

SEQ ID NO: 230 - DNA, 54 bp, Escherichia coli
  source 1..54: mol_type = genomic DNA
aaggttgaaaaataaaaacggcgctaaaaagcgccgttttttttgacggtggta

SEQ ID NO: 231 - DNA, 71 bp, Escherichia coli
  source 1..71: mol_type = genomic DNA
cgcaaataaccaggagataaaaccgaccacggcaccaggcagtgaccatgtggtttcttcatcctcagtaa

SEQ ID NO: 232 - DNA, 51 bp, Escherichia coli
  source 1..51: mol_type = genomic DNA
ctctggtagtcctggtaagacgcgaacagcgtcgcatcaggcatattgcca

SEQ ID NO: 233 - DNA, 51 bp, Escherichia coli
  source 1..51: mol_type = genomic DNA
accggaaaacaaactaaagcgcccttgtggcgctttagttttgttcatctt

SEQ ID NO: 234 - DNA, 46 bp, Escherichia coli
  source 1..46: mol_type = genomic DNA
tgtaagagataaaaaaggccggagcatgctccggccttcgttttca

SEQ ID NO: 235 - DNA, 41 bp, Escherichia coli
  source 1..41: mol_type = genomic DNA
aacggtttattagtctggagacggcagactatcctcttccc

SEQ ID NO: 236 - DNA, 35 bp, Escherichia coli
  source 1..35: mol_type = genomic DNA
agacaaaaaaacccgccgcagcgggtctttgagcc

SEQ ID NO: 237 - DNA, 39 bp, Escherichia coli
  source 1..39: mol_type = genomic DNA
tgcataaaaaagccaacccgcaggttggcttttctcgtt

SEQ ID NO: 238 - DNA, 41 bp, Escherichia coli
  source 1..41: mol_type = genomic DNA
aaaaacaaaaaccccggactctcatccagggttctctgctt

SEQ ID NO: 239 - DNA, 36 bp, Escherichia coli
  source 1..36: mol_type = genomic DNA
aggggtaaaaccgtccaggagcagggaaaagggcag

SEQ ID NO: 240 - DNA, 47 bp, Escherichia coli
  source 1..47: mol_type = genomic DNA
gtcagcttgaaaaaaggggccactcaggcccccttttctgaaactcg

SEQ ID NO: 241 - DNA, 60 bp, Escherichia coli
  source 1..60: mol_type = genomic DNA
tgcggagggagtggaaacgcctcagccggaactgaccgaggcggtataacttaacgcagt

SEQ ID NO: 242 - DNA, 47 bp, Escherichia coli
  source 1..47: mol_type = genomic DNA
ggggaaataaacggcccatccatgaggaatgggccgtgaaaggagat

SEQ ID NO: 243 - DNA, 42 bp, Escherichia coli
  source 1..42: mol_type = genomic DNA
tggggagactaaggcagccagatggctgccttttttacaggt

SEQ ID NO: 244 - DNA, 44 bp, Escherichia coli
  source 1..44: mol_type = genomic DNA
agtgaaaagaaaaaaggccgcagagcggcctttttagttagatc

SEQ ID NO: 245 - DNA, 40 bp, Escherichia coli
  source 1..40: mol_type = genomic DNA
ttcctgacttaagcggcgctggttatccatcggagccatc

SEQ ID NO: 246 - DNA, 36 bp, Escherichia coli
  source 1..36: mol_type = genomic DNA
tccggcatgaacaacgcgcacgttgtcagcaatctg

SEQ ID NO: 247 - DNA, 56 bp, Escherichia coli
  source 1..56: mol_type = genomic DNA
aaaaatatgaatatattccggcgcttaatgccacgccggaacatatcgaaatgatg

SEQ ID NO: 248 - DNA, 48 bp, Escherichia coli
  source 1..48: mol_type = genomic DNA
ttgaagataaaaaaccctctgtagtaacagagggttttgttcattcat

SEQ ID NO: 249 - DNA, 45 bp, Escherichia coli
  source 1..45: mol_type = genomic DNA
gttaaaaaaatgcccggttgtgaaaagcaaccgggcatcattgtg

SEQ ID NO: 250 - DNA, 34 bp, Escherichia coli
  source 1..34: mol_type = genomic DNA
aaagtaaaaacccgccgaagcgggtttttacgta

SEQ ID NO: 251 - DNA, 40 bp, Escherichia coli
  source 1..40: mol_type = genomic DNA
aaaagaggtggccagggggatcacctggcagcatgctgcc

SEQ ID NO: 252 - DNA, 52 bp, Escherichia coli
  source 1..52: mol_type = genomic DNA
ccttcgctcatcccattcacatagttatctatgctcatgggagttcactcag

SEQ ID NO: 253 - DNA, 44 bp, Escherichia coli
  source 1..44: mol_type = genomic DNA
tttaaaagattatcgggagagttacctcccgatataaaaggaag

SEQ ID NO: 254 - DNA, 42 bp, Escherichia coli
  source 1..42: mol_type = genomic DNA
tccatacaaaacggcccgccataggcgggccggatttacatt

SEQ ID NO: 255 - DNA, 43 bp, Escherichia coli
  source 1..43: mol_type = genomic DNA
cgcaaaaaaaagccagcctgtttccagactggcttttgtgctt

SEQ ID NO: 256 - DNA, 43 bp, Escherichia coli
  source 1..43: mol_type = genomic DNA
tatcataaaagcagctctgaagagcagagccgcgaatcctttt

SEQ ID NO: 257 - DNA, 42 bp, Escherichia coli
  source 1..42: mol_type = genomic DNA
ccatcaaaaaaccaggcttgagtatagcctggtttcgtttga

SEQ ID NO: 258 - DNA, 34 bp, Escherichia coli
  source 1..34: mol_type = genomic DNA
aaagcaaaaacccgccgaagcgggtttttacgta

SEQ ID NO: 259 - DNA, 57 bp, Escherichia coli
  source 1..57: mol_type = genomic DNA
ggaccaaaacgaaaaaagacgcttttcagcgtctcttttctggaatttggtaccgag

SEQ ID NO: 260 - DNA, 60 bp, Escherichia coli
  source 1..60: mol_type = genomic DNA
gggtcaaagaggcatgatgcgacgcttgttcctgcgctttgttcatgccggatgcggcta

SEQ ID NO: 261 - DNA, 41 bp, Escherichia coli
  source 1..41: mol_type = genomic DNA
gctacaaaaatgcccgatcctcgatcgggcattttgacttt

SEQ ID NO: 262 - DNA, 41 bp, Escherichia coli
  source 1..41: mol_type = genomic DNA
aggaagaaaaatgccccgcttacgcagggcatccatttatt

SEQ ID NO: 263 - DNA, 50 bp, Escherichia coli
  source 1..50: mol_type = genomic DNA
gaacacatttgtcggatgcggcgcgagcgccttatccgacctacggttcg

SEQ ID NO: 264 - DNA, 41 bp, Escherichia coli
  source 1..41: mol_type = genomic DNA
tcatcagtctgagcctgccattggcaggctctggtgtcctt

SEQ ID NO: 265 - DNA, 39 bp, Escherichia coli
  source 1..39: mol_type = genomic DNA
gaaaaaataaacggctcatttctgagccgtttattcgta

SEQ ID NO: 266 - DNA, 47 bp, Escherichia coli
  source 1..47: mol_type = genomic DNA
taacaatcagaaatgccgggaataaatcccggcattttcataatcag

SEQ ID NO: 267 - DNA, 48 bp, Escherichia coli
  source 1..48: mol_type = genomic DNA
accaggtatagcagataaagagaggattatttcgctgcaatgagccgg

SEQ ID NO: 268 - DNA, 48 bp, Escherichia coli
  source 1..48: mol_type = genomic DNA
ttgataagaaaaaaccccggagcacgccccggggttttcggtacaaat

SEQ ID NO: 269 - DNA, 40 bp, Escherichia coli
  source 1..40: mol_type = genomic DNA
gaaacgaaaagagggagactagctccctctttcaactggc

SEQ ID NO: 270 - DNA, 51 bp, Escherichia coli
  source 1..51: mol_type = genomic DNA
aagaattaaaaatgttacgggtaactgttatcggtaacattgtccagccgg

SEQ ID NO: 271 - DNA, 47 bp, Escherichia coli
  source 1..47: mol_type = genomic DNA
ttttagctataaaaaaacccgccgaagcgggttttttcgaaaattgt

SEQ ID NO: 272 - DNA, 35 bp, Escherichia coli
  source 1..35: mol_type = genomic DNA
caatgaaaaaagggcccgcaggccctttgttcgat

SEQ ID NO: 273 - DNA, 47 bp, Escherichia coli
  source 1..47: mol_type = genomic DNA
aagcacgaaaaagggagcgatgaattatcgctcccttgtcttataac

SEQ ID NO: 274 - DNA, 37 bp, Escherichia coli
  source 1..37: mol_type = genomic DNA
ataacaaaaaaccccgccggagcgaggttttttgtta

SEQ ID NO: 275 - DNA, 48 bp, Escherichia coli
  source 1..48: mol_type = genomic DNA
agtaaataaatctggcggagcctgggagctccgccagagccgttaaac

SEQ ID NO: 276 - DNA, 36 bp, Escherichia coli
  source 1..36: mol_type = genomic DNA
aaaaacaaaaacccgccgaagcgggttaagtgcggg

SEQ ID NO: 277 - DNA, 42 bp, Escherichia coli
  source 1..42: mol_type = genomic DNA
aacaaaaaaattccagtcccgaaggactggaaggctcaatcg

SEQ ID NO: 278 - DNA, 51 bp, Escherichia coli
  source 1..51: mol_type = genomic DNA
agtaagaacgaaaaaggccgcagagtttcccccctgcggcctggtccgggc

SEQ ID NO: 279 - DNA, 36 bp, Escherichia coli
  source 1..36: mol_type = genomic DNA
tacaaaaaaagcctccactgggaggctttcaggcgc

SEQ ID NO: 280 - DNA, 42 bp, Escherichia coli
  source 1..42: mol_type = genomic DNA
tgcaaaaaaatcgggcagcgtcgtgctgcccgtgtgcatact

SEQ ID NO: 281 - DNA, 80 bp, Escherichia coli
  source 1..80: mol_type = genomic DNA
tcgcgctaaaaaaggccccactttcgtggagcctttattgtacgctttttactgtacgatttcagtcaaatctaattaca

SEQ ID NO: 282 - DNA, 52 bp, Escherichia coli
  source 1..52: mol_type = genomic DNA
caatccatgtaaaaaaagggccctgaaattcaggaccctttctggcatcagc

SEQ ID NO: 283 - DNA, 45 bp, Escherichia coli
  source 1..45: mol_type = genomic DNA
aaccatgaaggcccgataagcgcagcgcatcgggcaatttagcat

SEQ ID NO: 284 - DNA, 47 bp, Escherichia coli
  source 1..47: mol_type = genomic DNA
tagggaaaaatgcctgatagcgcttcgcttatcaggcctacccgcgc

SEQ ID NO: 285 - DNA, 39 bp, Escherichia coli
  source 1..39: mol_type = genomic DNA
ttcagaaaaaccgccgagcatgtcggcggctttctgact

SEQ ID NO: 286 - DNA, 62 bp, Escherichia coli
  source 1..62: mol_type = genomic DNA
tataaacaaaaaaacccgacttcaccagtattctctggttatgtcaggttttgcctgcgaat

SEQ ID NO: 287 - DNA, 51 bp, Escherichia coli
  source 1..51: mol_type = genomic DNA
acgcgtaaataaaaaaggcgctagtgaaagcgcccttttttgtcattatgc

SEQ ID NO: 288 - DNA, 56 bp, Escherichia coli
  source 1..56: mol_type = genomic DNA
tcaggttcccgtaggcatgataagacgcgtaagcgtcgcatcaggcaatgaatacc

SEQ ID NO: 289 - DNA, 51 bp, Escherichia coli
  source 1..51: mol_type = genomic DNA
attttgtaatgccggatgcggcgtaaaacaccgtacccggcctggagtttt

SEQ ID NO: 290 - DNA, 43 bp, Escherichia coli
  source 1..43: mol_type = genomic DNA
tcggtcggtcccctcgcccctctggggagagggttagggtgag

SEQ ID NO: 291 - DNA, 45 bp, Escherichia coli
  source 1..45: mol_type = genomic DNA
tataaaaattgcctgatacgctgcgcttatcaggcctacaagttc

SEQ ID NO: 292 - DNA, 40 bp, Escherichia coli
  source 1..40: mol_type = genomic DNA
cgataaaaaaagcctgccagatggcaggctatttaataac

SEQ ID NO: 293 - DNA, 72 bp, Escherichia coli
  source 1..72: mol_type = genomic DNA
actccaaaaaaaaacctgaaaaaaacggcctgacgtgaatcaagcaatttttttcaggttttgcccgcttag

SEQ ID NO: 294 - DNA, 46 bp, Escherichia coli
  source 1..46: mol_type = genomic DNA
aataaaaaaagggagcactgtattcacagcgctcccggttcgtttc

SEQ ID NO: 295 - DNA, 37 bp, Escherichia coli
  source 1..37: mol_type = genomic DNA
agaaaaaaaggcgctccgtggagcgccgaataacagt

SEQ ID NO: 296 - DNA, 33 bp, Escherichia coli
  source 1..33: mol_type = genomic DNA
cataaaaaaagggcctaaagcccagttattctg

SEQ ID NO: 297 - DNA, 34 bp, Escherichia coli
  source 1..34: mol_type = genomic DNA
cataaaaaaacccgcttgcgcgggctttttcaca

SEQ ID NO: 298 - DNA, 49 bp, Escherichia coli
  source 1..49: mol_type = genomic DNA
tgattatgcgagcagactcgcactcctgccagtctgctgcaaaagaaag

SEQ ID NO: 299 - DNA, 40 bp, Escherichia coli
  source 1..40: mol_type = genomic DNA
cgctgaaacaagggcaggtttccctgccctgtgatttttt

SEQ ID NO: 300 - DNA, 52 bp, Escherichia coli
  source 1..52: mol_type = genomic DNA
cgtatcagaccaggcagggtaaacagacacttcccctgcctgcatccgatta

SEQ ID NO: 301 - DNA, 44 bp, Escherichia coli
  source 1..44: mol_type = genomic DNA
tcgaaaagaacccggtagcgtcacaactaccgggcgaacaaact

SEQ ID NO: 302 - DNA, 42 bp, Escherichia coli
  source 1..42: mol_type = genomic DNA
aggccaaaaaaaaccggcgcaatggccggtttccgttgttac

SEQ ID NO: 303 - DNA, 48 bp, Escherichia coli
  source 1..48: mol_type = genomic DNA
gtaatcggtattatgccggatgctggcgcatccggcatgggttttact

SEQ ID NO: 304 - DNA, 38 bp, Escherichia coli
  source 1..38: mol_type = genomic DNA
gcggagaaaaggtccgaaaattcggacccgatggaatt

SEQ ID NO: 305 - DNA, 37 bp, Escherichia coli
  source 1..37: mol_type = genomic DNA
atttttttaacgggggaggttcccccgtcagatcatt

SEQ ID NO: 306 - DNA, 53 bp, Escherichia coli
  source 1..53: mol_type = genomic DNA
catgactaaaaacagcagcagtaaaacagaccctactgctgttaaaacaagcg

SEQ ID NO: 307 - DNA, 53 bp, Escherichia coli
  source 1..53: mol_type = genomic DNA
gtgaataaaacaaccggaccccatcgaggaacggagtccggtgtcatattaaa

SEQ ID NO: 308 - DNA, 64 bp, Escherichia coli
  source 1..64: mol_type = genomic DNA
aaagaaagaattaacgcatcgcatccagactgttctgaatgcgacgataattaaggtgctttaa

SEQ ID NO: 309 - DNA, 49 bp, Escherichia coli
  source 1..49: mol_type = genomic DNA
taataatccagtgccggatgattcacatcatccggcaccttttcatcag

SEQ ID NO: 310 - DNA, 39 bp, Escherichia coli
  source 1..39: mol_type = genomic DNA
taacgtagaaaggcttcccgaaggaagccttgatgatca

SEQ ID NO: 311 - DNA, 36 bp, Escherichia coli
  source 1..36: mol_type = genomic DNA
taacaaaaaaccccgataaatcggggctttatataa

SEQ ID NO: 312 - DNA, 45 bp, Escherichia coli
  source 1..45: mol_type = genomic DNA
aaagattattgccctcaccctgtacgggtgagggcgtagagagat

SEQ ID NO: 313 - DNA, 37 bp, Escherichia coli
  source 1..37: mol_type = genomic DNA
aacaaataagtgagagctgtaactctcgcttttctta

SEQ ID NO: 314 - DNA, 41 bp, Escherichia coli
  source 1..41: mol_type = genomic DNA
aagtgaaaaagccccggcacgataccggggcgaggcgatta

SEQ ID NO: 315 - DNA, 39 bp, Escherichia coli
  source 1..39: mol_type = genomic DNA
tggaaattaatgcgctggcggcaatggcacagcacagaa

SEQ ID NO: 316 - DNA, 38 bp, Escherichia coli
  source 1..38: mol_type = genomic DNA
ccaataaaaatggggcctttcagccccatcaaacaatg

SEQ ID NO: 317 - DNA, 38 bp, Escherichia coli
  source 1..38: mol_type = genomic DNA
aaaagcaaaagggccgcagatgcgacccttgtgtatca

SEQ ID NO: 318 - DNA, 61 bp, synthetic construct
  source 1..61: mol_type = other DNA
ctcggtaccaaattccagaaaagaggcctcccgaaaggggggccttttttcgttttggtcc

SEQ ID NO: 319 - DNA, 57 bp, synthetic construct
  source 1..57: mol_type = other DNA
ctcggtaccaaattttcgaaaaaagacgctgaaaagcgtcttttttcgttttggtcc

SEQ ID NO: 320 - DNA, 57 bp, synthetic construct
  source 1..57: mol_type = other DNA
ctcggtaccaaaaaaaaaaaaaaagacgctgaaaagcgtcttttttcgttttggtcc

SEQ ID NO: 321 - DNA, 52 bp, synthetic construct
  source 1..52: mol_type = other DNA
ttttcgaaaaaaggcctcccaaatcggggggccttttttattgataacaaaa

SEQ ID NO: 322 - DNA, 47 bp, synthetic construct
  source 1..47: mol_type = other DNA
aaaaaaaaaaaacaccctaacgggtgtttttttttttttggtctccc

SEQ ID NO: 323 - DNA, 47 bp, synthetic construct
  source 1..47: mol_type = other DNA
aaaaaaaaaaaacaccctaacgggtgtttttttgtttctggtctccc

SEQ ID NO: 324 - DNA, 52 bp, synthetic construct
  source 1..52: mol_type = other DNA
tctaactaaaaaggcctcccaaatcggggggccttttttattgataacaaaa

SEQ ID NO: 325 - DNA, 57 bp, synthetic construct
  source 1..57: mol_type = other DNA
ctcggtaccaaaaaaaaaaaaaaagacgctgaaaagcgtcttttttttttttggtcc

SEQ ID NO: 326 - DNA, 57 bp, synthetic construct
  source 1..57: mol_type = other DNA
ctcggtaccaaattccagaaaagaggccgcgaaagcggccttttttcgttttggtcc

SEQ ID NO: 327 - DNA, 57 bp, synthetic construct
  source 1..57: mol_type = other DNA
ctcggtaccaaattccagaaaagagacgctttcgagcgtcttttttcgttttggtcc

SEQ ID NO: 328 - DNA, 57 bp, synthetic construct
  source 1..57: mol_type = other DNA
ctcggtaccaaagacgaacaataagacgctgaaaagcgtcttttttcgttttggtcc

SEQ ID NO: 329 - DNA, 53 bp, synthetic construct
  source 1..53: mol_type = other DNA
ccaattattgaaggcctccctaacggggggcctttttttgtttctggtctccc

SEQ ID NO: 330 - DNA, 47 bp, synthetic construct
  source 1..47: mol_type = other DNA
ttttcgaaaaaacaccctaacgggtgtttttttgtttctggtctccc

SEQ ID NO: 331 - DNA, 57 bp, synthetic construct
  source 1..57: mol_type = other DNA
ctcggtaccaaatctaactaaaaagacgctgaaaagcgtcttttttcgttttggtcc

SEQ ID NO: 332 - DNA, 52 bp, synthetic construct
  source 1..52: mol_type = other DNA
aaaaaaaaaaaaggcctcccaaatcggggggccttttttattgataacaaaa

SEQ ID NO: 333 - DNA, 57 bp, synthetic construct
  source 1..57: mol_type = other DNA
ctcggtaccaaattccagaaaagagacgcttaacagcgtcttttttcgttttggtcc

SEQ ID NO: 334 - DNA, 49 bp, synthetic construct
  source 1..49: mol_type = other DNA
ccaattattgaaggccgctaacgcggcctttttttgtttctggtctccc

SEQ ID NO: 335 - DNA, 52 bp, synthetic construct
  source 1..52: mol_type = other DNA
ccaattattgaaggcctcccaaatcggggggccttttttattgataacaaaa

SEQ ID NO: 336 - DNA, 55 bp, synthetic construct
  source 1..55: mol_type = other DNA
ccaattattgaaggggagcgggaaaccgctcccctttttttgtttctggtctccc

SEQ ID NO: 337 - DNA, 57 bp, synthetic construct
  source 1..57: mol_type = other DNA
ctcggtaccaaaccaattattgaagacgctgaaaagcgtcttttttcgttttggtcc

SEQ ID NO: 338 - DNA, 58 bp, synthetic construct
  source 1..58: mol_type = other DNA
ctcggtaccaaattccagaaaagagacgcttttagagcgtcttttttcgttttggtcc

SEQ ID NO: 339 - DNA, 57 bp, synthetic construct
  source 1..57: mol_type = other DNA
ctcggtaccaaattccagaaaagagacgctgaaaagcgtcttttttttttttggtcc

SEQ ID NO: 340 - DNA, 51 bp, synthetic construct
  source 1..51: mol_type = other DNA
gacgaacaataaggcctccctaacggggggccttttttattgataacaaaa

SEQ ID NO: 341 - DNA, 47 bp, synthetic construct
  source 1..47: mol_type = other DNA
ccaattattgaacacccttcggggtgtttttttgtttctggtctccc

SEQ ID NO: 342 - DNA, 49 bp, synthetic construct
  source 1..49: mol_type = other DNA
ccaattattgaagacgcttaacagcgtctttttttgtttctggtctccc

SEQ ID NO: 343 - DNA, 51 bp, synthetic construct
  source 1..51: mol_type = other DNA
gacgaacaataaggcctcccgaaaggggggccttttttattgataacaaaa

SEQ ID NO: 344 - DNA, 47 bp, synthetic construct
  source 1..47: mol_type = other DNA
tctaactaaaaacaccctaacgggtgtttttttgtttctggtctccc

SEQ ID NO: 345 - DNA, 47 bp, synthetic construct
  source 1..47: mol_type = other DNA
ccaattattgaacacccgaaagggtgtttttttgtttctggtctccc

SEQ ID NO: 346 - DNA, 52 bp, synthetic construct
  source 1..52: mol_type = other DNA
aaaaaaaaaaaaggcctcccaaatcggggggccttttttttttttaacaaaa

SEQ ID NO: 347 - DNA, 55 bp, synthetic construct
  source 1..55: mol_type = other DNA
ctcggtaccaaattccagaaaagacacccgaaagggtgttttttcgttttggtcc

SEQ ID NO: 348 - DNA, 58 bp, synthetic construct
  source 1..58: mol_type = other DNA
ctcggtaccaaattccagaaaagagacgctaaatcagcgtcttttttcgttttggtcc

SEQ ID NO: 349 - DNA, 52 bp, synthetic construct
  source 1..52: mol_type = other DNA
ttccagaaaagaggcctcccaaatcggggggccttttttattgataacaaaa

SEQ ID NO: 350 - DNA, 48 bp, synthetic construct
  source 1..48: mol_type = other DNA
gacgaacaataaggccgcaaatcgcggccttttttattgataacaaaa

SEQ ID NO: 351 - DNA, 63 bp, synthetic construct
  source 1..63: mol_type = other DNA
ctcggtaccaaattccagaaaagaggggagcgggaaaccgctccccttttttcgttttggtcc

SEQ ID NO: 352 - DNA, 52 bp, synthetic construct
  source 1..52: mol_type = other DNA
ttttcgaaaaaaggcctcccaaatcggggggcctttttttatagcaacaaaa

SEQ ID NO: 353 - DNA, 47 bp, synthetic construct
  source 1..47: mol_type = other DNA
ttccagaaaagacaccctaacgggtgttttttcgtttttggtctccc

SEQ ID NO: 354 - DNA, 57 bp, synthetic construct
  source 1..57: mol_type = other DNA
ctcggtaccaaaccaattattgaagacgctgaaaagcgtctttttttgtttcggtcc

SEQ ID NO: 355 - DNA, 47 bp, synthetic construct
  source 1..47: mol_type = other DNA
ttccagaaaagacaccctaacgggtgtttttttgtttctggtctccc

SEQ ID NO: 356 - DNA, 47 bp, synthetic construct
  source 1..47: mol_type = other DNA
ccaattattgaacaccctaacgggtgtttttttttttttggtctccc

SEQ ID NO: 357 - DNA, 51 bp, synthetic construct
  source 1..51: mol_type = other DNA
gacgaacaataaggcctcccttcgggggggccttttttattgataacaaaa

SEQ ID NO: 358 - DNA, 47 bp, synthetic construct
  source 1..47: mol_type = other DNA
tctaactaaaaacaccctaacgggtgttttttcttttctggtctccc

SEQ ID NO: 359 - DNA, 47 bp, synthetic construct
  source 1..47: mol_type = other DNA
gaaaaataaaaacaccctaacgggtgtttttatttttctggtctccc

SEQ ID NO: 360 - DNA, 57 bp, synthetic construct
  source 1..57: mol_type = other DNA
ctcggtaccaaagaaaaataaaaagacgctgaaaagcgtctttttatttttcggtcc

SEQ ID NO: 361 - DNA, 48 bp, synthetic construct
  source 1..48: mol_type = other DNA
ccaattattgaacacccaaatcgggtgtttttttgtttctggtctccc

SEQ ID NO: 362 - DNA, 57 bp, synthetic construct
  source 1..57: mol_type = other DNA
ctcggtaccaaatctaactaaaaagacgctgaaaagcgtcttttttcttttcggtcc

SEQ ID NO: 363 - DNA, 47 bp, synthetic construct
  source 1..47: mol_type = other DNA
ccaattattgaacaccctaacgggtgttttttcgtttttggtctccc

SEQ ID NO: 364 - DNA, 53 bp, synthetic construct
  source 1..53: mol_type = other DNA
gacgaacaataaggggagcgggaaaccgctccccttttttattgataacaaaa

SEQ ID NO: 365 - DNA, 47 bp, synthetic construct
  source 1..47: mol_type = other DNA
ttttcgaaaaaacaccctaacgggtgtttttttatagctggtctccc

SEQ ID NO: 366 - DNA, 52 bp, synthetic construct
  source 1..52: mol_type = other DNA
gaaaaataaaaaggcctcccaaatcggggggcctttttatttttcaacaaaa

SEQ ID NO: 367 - DNA, 48 bp, synthetic construct
  source 1..48: mol_type = other DNA
ccaattattgaacaccctttaggggtgtttttttgtttctggtctccc

SEQ ID NO: 368 - DNA, 57 bp, synthetic construct
  source 1..57: mol_type = other DNA
ctcggtaccaaattttcgaaaaaagacgctgaaaagcgtctttttttatagcggtcc

SEQ ID NO: 369 - DNA, 52 bp, synthetic construct
  source 1..52: mol_type = other DNA
gacgaacaataaggcctcccaaatcggggggcctttttttgtttcaacaaaa

SEQ ID NO: 370 - DNA, 57 bp, synthetic construct
  source 1..57: mol_type = other DNA
ctcggtaccaaattccagaaaagagacgctgaaaagcgtctttttttgtttcggtcc

SEQ ID NO: 371 - DNA, 47 bp, synthetic construct
  source 1..47: mol_type = other DNA
ccaattattgaacaccctaacgggtgttttttcttttctggtctccc

SEQ ID NO: 372 - DNA, 47 bp, synthetic construct
  source 1..47: mol_type = other DNA
ccaattattgaagcggctaacgccgctttttttgtttctggtctccc

SEQ ID NO: 373 - DNA, 57 bp, synthetic construct
  source 1..57: mol_type = other DNA
ctcggtaccaaattccagaaaagagacgctgaaaagcgtcttttttcttttcggtcc

SEQ ID NO: 374 - DNA, 47 bp, synthetic construct
  source 1..47: mol_type = other DNA
gacgaacaataacaccctaacgggtgtttttttgtttctggtctccc

SEQ ID NO: 375 - DNA, 47 bp, synthetic construct
  source 1..47: mol_type = other DNA
ccaattattgaacaccctaacgggtgtttttatttttctggtctccc

SEQ ID NO: 376 - DNA, 52 bp, synthetic construct
  source 1..52: mol_type = other DNA
ccaattattgaaggcctcccaaatcggggggcctttttttgtttcaacaaaa

SEQ ID NO: 377 - DNA, 52 bp, synthetic construct
  source 1..52: mol_type = other DNA
tctaactaaaaaggcctcccaaatcggggggccttttttcttttcaacaaaa

SEQ ID NO: 378 - DNA, 47 bp, synthetic construct
  source 1..47: mol_type = other DNA
ccaattattgaacaccctaacgggtgtttttttatagctggtctccc

SEQ ID NO: 379 - DNA, 57 bp, synthetic construct
  source 1..57: mol_type = other DNA
ctcggtaccaaattccagaaaagagacgctgaaaagcgtctttttttatagcggtcc

SEQ ID NO: 380 - DNA, 52 bp, synthetic construct
  source 1..52: mol_type = other DNA
gacgaacaataaggcctcccaaatcggggggcctttttatttttcaacaaaa

SEQ ID NO: 381 - DNA, 57 bp, synthetic construct
  source 1..57: mol_type = other DNA
ctcggtaccaaagacgaacaataagacgctgaaaagcgtcttttttattgatggtcc

SEQ ID NO: 382 - DNA, 52 bp, synthetic construct
  source 1..52: mol_type = other DNA
gacgaacaataaggcctcccaaatcggggggccttttttttttttaacaaaa

SEQ ID NO: 383 - DNA, 52 bp, synthetic construct
  source 1..52: mol_type = other DNA
gacgaacaataaggcctcccaaatcggggggccttttttcttttcaacaaaa

SEQ ID NO: 384 - DNA, 52 bp, synthetic construct
  source 1..52: mol_type = other DNA
ttccagaaaagaggcctcccaaatcggggggccttttttcgttttaacaaaa

SEQ ID NO: 385 - DNA, 57 bp, synthetic construct
  source 1..57: mol_type = other DNA
ctcggtaccaaattccagaaaagagacgctgaaaagcgtcttttttattgatggtcc

SEQ ID NO: 386 - DNA, 29 bp, synthetic construct
  source 1..29: mol_type = other DNA
gggcggtcagatgatcgcccttttttttt

SEQ ID NO: 387 - DNA, 29 bp, synthetic construct
  source 1..29: mol_type = other DNA
acggccctagatagggccgtttttttttt

SEQ ID NO: 388 - DNA, 52 bp, synthetic construct
  source 1..52: mol_type = other DNA
gacgaacaataaggcctccctttagggggggccttttttattgataacaaaa

SEQ ID NO: 389 - DNA, 52 bp, synthetic construct
  source 1..52: mol_type = other DNA
gacgaacaataaggcctcccaaatcggggggccttttttcgttttaacaaaa

SEQ ID NO: 390 - DNA, 23 bp, synthetic construct
  source 1..23: mol_type = other DNA
cccgcttcggcgggttttttttt

SEQ ID NO: 391 - DNA, 35 bp, synthetic construct
  source 1..35: mol_type = other DNA
gatccagcccattcgtgggctggatcttttttttt

SEQ ID NO: 392 - DNA, 48 bp, synthetic construct
  source 1..48: mol_type = other DNA
gacgaacaataagacgctaaatcagcgtcttttttattgataacaaaa

SEQ ID NO: 393 - DNA, 46 bp, synthetic construct
  source 1..46: mol_type = other DNA
gacgaacaataagcggcaaatcgccgcttttttattgataacaaaa

SEQ ID NO: 394 - DNA, 57 bp, synthetic construct
  source 1..57: mol_type = other DNA
ctcggtaccaaattccagaaaagagacgctgaaaagcgtctttttatttttcggtcc

SEQ ID NO: 395 - DNA, 47 bp, synthetic construct
  source 1..47: mol_type = other DNA
gacgaacaataacaccctaacgggtgttttttattgattggtctccc

SEQ ID NO: 396 - DNA, 31 bp, synthetic construct
  source 1..31: mol_type = other DNA
aggcctcccagatggggggcctttttttttt

SEQ ID NO: 397 - DNA, 35 bp, synthetic construct
  source 1..35: mol_type = other DNA
cccgcatgccattcgtggcatgcgggttttctctg

SEQ ID NO: 398 - DNA, 29 bp, synthetic construct
  source 1..29: mol_type = other DNA
gatccagcttcggctggatcttttctctg

SEQ ID NO: 399 - DNA, 23 bp, synthetic construct
  source 1..23: mol_type = other DNA
cccgcttcggcgggttttctctg

SEQ ID NO: 400 - DNA, 29 bp, synthetic construct
  source 1..29: mol_type = other DNA
cccgcatgttcgcatgcgggttttctctg

SEQ ID NO: 401 - DNA, 35 bp, synthetic construct
  source 1..35: mol_type = other DNA
tcggttaccgcttcggcggtaaccgattttctctg

SEQ ID NO: 402 - DNA, 35 bp, synthetic construct
  source 1..35: mol_type = other DNA
tcggttacatgttcgcatgtaaccgattttctctg

SEQ ID NO: 403 - DNA, 29 bp, synthetic construct
  source 1..29: mol_type = other DNA
tgccgggaagattcccggcattttttttt

SEQ ID NO: 404 - DNA, 27 bp, synthetic construct
  source 1..27: mol_type = other DNA
cgcccgcagatgcgggcgttttttttt

SEQ ID NO: 405 - DNA, 35 bp, synthetic construct
  source 1..35: mol_type = other DNA
gatccagcccattcgtgggctggatcttttctctg

SEQ ID NO: 406 - DNA, 35 bp, synthetic construct
  source 1..35: mol_type = other DNA
cgacgatgccattcgtggcatcgtcgttttctctg

SEQ ID NO: 407 - DNA, 29 bp, synthetic construct
  source 1..29: mol_type = other DNA
cgacgatgttcgcatcgtcgttttctctg

SEQ ID NO: 408 - DNA, 46 bp, synthetic construct
  source 1..46: mol_type = other DNA
gacgaacaataacacccaaatcgggtgttttttattgataacaaaa

SEQ ID NO: 409 - DNA, 35 bp, synthetic construct
  source 1..35: mol_type = other DNA
cgacgataccattcgtggtatcgtcgttttctctg

SEQ ID NO: 410 - DNA, 36 bp, synthetic construct
  source 1..36: mol_type = other DNA
ctaaagcgcctaaccacggcgctttagttttttgta

SEQ ID NO: 411 - DNA, 33 bp, synthetic construct
  source 1..33: mol_type = other DNA
ctaaagcgccagatggcgctttagttttttttt

SEQ ID NO: 412 - DNA, 29 bp, synthetic construct
  source 1..29: mol_type = other DNA
acggccctcgcaagggccgtttttttgta

SEQ ID NO: 413 - DNA, 32 bp, synthetic construct
  source 1..32: mol_type = other DNA
acggcccttaaccacagggccgtttttttgta

SEQ ID NO: 414 - DNA, 33 bp, synthetic construct
  source 1..33: mol_type = other DNA
ctaaagcgcccgcaggcgctttagttttttgta

SEQ ID NO: 415 - DNA, 29 bp, synthetic construct
  source 1..29: mol_type = other DNA
gatccagcttcggctggatcttttttttt

SEQ ID NO: 416 - DNA, 34 bp, synthetic construct
  source 1..34: mol_type = other DNA
aggcctccctaaccacggggggcctttttttgta

SEQ ID NO: 417 - DNA, 23 bp, synthetic construct
  source 1..23: mol_type = other DNA
cgacgttcgcgtcgttttctctg

SEQ ID NO: 418 - DNA, 29 bp, synthetic construct
  source 1..29: mol_type = other DNA
cccgcatgttcgcatgcgggttttttttt

SEQ ID NO: 419 - DNA, 32 bp, synthetic construct
  source 1..32: mol_type = other DNA
aggcctcccgaacaggggggcctttttgcaac

SEQ ID NO: 420 - DNA, 29 bp, synthetic construct
  source 1..29: mol_type = other DNA
cgacgatattcgtatcgtcgttttctctg

SEQ ID NO: 421 - DNA, 29 bp, synthetic construct
  source 1..29: mol_type = other DNA
tagcgtgcagatgcacgctattttttttt

SEQ ID NO: 422 - DNA, 35 bp, synthetic construct
  source 1..35: mol_type = other DNA
gatccagcccattcgtgggctggatcttttttggg

SEQ ID NO: 423 - DNA, 35 bp, synthetic construct
  source 1..35: mol_type = other DNA
gatccagcccattcgtgggctggatctttattgtc

SEQ ID NO: 424 - DNA, 29 bp, synthetic construct
  source 1..29: mol_type = other DNA
acggccctcgcaagggccgtttttgcaac

SEQ ID NO: 425 - DNA, 33 bp, synthetic construct
  source 1..33: mol_type = other DNA
gccggagcggagatctgctccggcttttttttt

SEQ ID NO: 426 - DNA, 29 bp, synthetic construct
  source 1..29: mol_type = other DNA
gatccagcttcggctggatcttttgttgg

SEQ ID NO: 427 - DNA, 30 bp, synthetic construct
  source 1..30: mol_type = other DNA
cgcccgctaaccacgcgggcgttttttgta

SEQ ID NO: 428 - DNA, 34 bp, synthetic construct
  source 1..34: mol_type = other DNA
ctaaagcgccgaacaggcgctttagttttgcaac

SEQ ID NO: 429 - DNA, 35 bp, synthetic construct
  source 1..35: mol_type = other DNA
cccgcatgccattcgtggcatgcgggtttattgtc

SEQ ID NO: 430 - DNA, 35 bp, synthetic construct
  source 1..35: mol_type = other DNA
tcggttaccgcttcggcggtaaccgattttttggg

SEQ ID NO: 431 - DNA, 29 bp, synthetic construct
  source 1..29: mol_type = other DNA
gatccagcttcggctggatctttattgtc

SEQ ID NO: 432 - DNA, 35 bp, synthetic construct
  source 1..35: mol_type = other DNA
cgacgatgccattcgtggcatcgtcgttttttggg

SEQ ID NO: 433 - DNA, 30 bp, synthetic construct
  source 1..30: mol_type = other DNA
acggccctgaacaagggccgtttttgcaac

SEQ ID NO: 434 - DNA, 29 bp, synthetic construct
  source 1..29: mol_type = other DNA
cgacgatgttcgcatcgtcgtttattgtc

SEQ ID NO: 435 - DNA, 27 bp, synthetic construct
  source 1..27: mol_type = other DNA
cgcccgccgcagcgggcgttttttgta

SEQ ID NO: 436 - DNA, 29 bp, synthetic construct
  source 1..29: mol_type = other DNA
cgacgatgttcgcatcgtcgttttttttt

SEQ ID NO: 437 - DNA, 35 bp, synthetic construct
  source 1..35: mol_type = other DNA
tcggttaccgcttcggcggtaaccgatttattgtc

SEQ ID NO: 438 - DNA, 29 bp, synthetic construct
  source 1..29: mol_type = other DNA
tagcgtgccgcagcacgctattttttgta

SEQ ID NO: 439 - DNA, 27 bp, synthetic construct
  source 1..27: mol_type = other DNA
cgcccgccgcagcgggcgttttgcaac

SEQ ID NO: 440 - DNA, 29 bp, synthetic construct
  source 1..29: mol_type = other DNA
gatccagcttcggctggatcttttttggg

SEQ ID NO: 441 - DNA, 29 bp, synthetic construct
  source 1..29: mol_type = other DNA
gggcggtccgcagatcgcccttttgcaac

SEQ ID NO: 442 - DNA, 29 bp, synthetic construct
  source 1..29: mol_type = other DNA
acggccctcgcaagggccgttttgttgag

SEQ ID NO: 443 - DNA, 29 bp, synthetic construct
  source 1..29: mol_type = other DNA
cccgcatgttcgcatgcgggtttattgtc

SEQ ID NO: 444 - DNA, 23 bp, synthetic construct
  source 1..23: mol_type = other DNA
cccgcttcggcgggttttttggg

SEQ ID NO: 445 - DNA, 35 bp, synthetic construct
  source 1..35: mol_type = other DNA
cgacgatgccattcgtggcatcgtcgtttattgtc

SEQ ID NO: 446 - DNA, 35 bp, synthetic construct
  source 1..35: mol_type = other DNA
cccgcatgccattcgtggcatgcgggttttttggg

SEQ ID NO: 447 - DNA, 35 bp, synthetic construct
  source 1..35: mol_type = other DNA
tcggttacatgttcgcatgtaaccgatttattgtc

SEQ ID NO: 448 - DNA, 35 bp, synthetic construct
  source 1..35: mol_type = other DNA
tcggttacatgttcgcatgtaaccgattttttttt

SEQ ID NO: 449 - DNA, 35 bp, synthetic construct
  source 1..35: mol_type = other DNA
cgacgataccattcgtggtatcgtcgtttattgtc

SEQ ID NO: 450 - DNA, 23 bp, synthetic construct
  source 1..23: mol_type = other DNA
cgacgttcgcgtcgttaaaataa

SEQ ID NO: 451 - DNA, 36 bp, synthetic construct
  source 1..36: mol_type = other DNA
gccggagcggtaaccacctgctccggcttttttgta

SEQ ID NO: 452 - DNA, 32 bp, synthetic construct
  source 1..32: mol_type = other DNA
tagcgtgctaaccacgcacgctattttttgta

SEQ ID NO: 453 - DNA, 32 bp, synthetic construct
  source 1..32: mol_type = other DNA
gggcggtctaaccacgatcgcccttttttgta

SEQ ID NO: 454 - DNA, 31 bp, synthetic construct
  source 1..31: mol_type = other DNA
aggcctccccgcaggggggcctttttttgta

SEQ ID NO: 455 - DNA, 33 bp, synthetic construct
  source 1..33: mol_type = other DNA
ctaaagcgcccgcaggcgctttagttttgcaac

SEQ ID NO: 456 - DNA, 33 bp, synthetic construct
  source 1..33: mol_type = other DNA
gccggatcggagatctgatccggcttttttttt

SEQ ID NO: 457 - DNA, 35 bp, synthetic construct
  source 1..35: mol_type = other DNA
tcggttaccgcttcggcggtaaccgattttttttt

SEQ ID NO: 458 - DNA, 29 bp, synthetic construct
  source 1..29: mol_type = other DNA
cgacgatattcgtatcgtcgtttattgtc

SEQ ID NO: 459 - DNA, 29 bp, synthetic construct
  source 1..29: mol_type = other DNA
cccgcatgttcgcatgcgggttttttggg

SEQ ID NO: 460 - DNA, 35 bp, synthetic construct
  source 1..35: mol_type = other DNA
cgacgataccattcgtggtatcgtcgttttttggg

SEQ ID NO: 461 - DNA, 29 bp, synthetic construct
  source 1..29: mol_type = other DNA
tgccgggacgcatcccggcattttttgta

SEQ ID NO: 462 - DNA, 31 bp, synthetic construct
  source 1..31: mol_type = other DNA
aggcctccccgcaggggggccttttgttgag

SEQ ID NO: 463 - DNA, 32 bp, synthetic construct
  source 1..32: mol_type = other DNA
tgccgggataaccactcccggcattttttgta

SEQ ID NO: 464 - DNA, 36 bp, synthetic construct
  source 1..36: mol_type = other DNA
gccggatcggtaaccacctgatccggcttttttgta

SEQ ID NO: 465 - DNA, 35 bp, synthetic construct
  source 1..35: mol_type = other DNA
gatccagcccattcgtgggctggatcttttgttgg

SEQ ID NO: 466 - DNA, 37 bp, synthetic construct
  source 1..37: mol_type = other DNA
tagcgtgaccggagattcggtcacgctattttttttt

SEQ ID NO: 467 - DNA, 29 bp, synthetic construct
  source 1..29: mol_type = other DNA
cgacgatgttcgcatcgtcgttttttggg

SEQ ID NO: 468 - DNA, 23 bp, synthetic construct
  source 1..23: mol_type = other DNA
cgacgttcgcgtcgtttattgtc

SEQ ID NO: 469 - DNA, 35 bp, synthetic construct
  source 1..35: mol_type = other DNA
cgacgatgccattcgtggcatcgtcgttttttttt

SEQ ID NO: 470 - DNA, 35 bp, synthetic construct
  source 1..35: mol_type = other DNA
cccgcatgccattcgtggcatgcgggttttgttgg

SEQ ID NO: 471 - DNA, 35 bp, synthetic construct
  source 1..35: mol_type = other DNA
tcggttacatgttcgcatgtaaccgattttttggg

SEQ ID NO: 472 - DNA, 34 bp, synthetic construct
  source 1..34: mol_type = other DNA
gccggatcgggaacactgatccggcttttgcaac

SEQ ID NO: 473 - DNA, 35 bp, synthetic construct
  source 1..35: mol_type = other DNA
cccgcatgccattcgtggcatgcgggttttttttt

SEQ ID NO: 474 - DNA, 29 bp, synthetic construct
  source 1..29: mol_type = other DNA
cgacgatattcgtatcgtcgttttttggg

SEQ ID NO: 475 - DNA, 28 bp, synthetic construct
  source 1..28: mol_type = other DNA
cgcccgcgaacagcgggcgttttgcaac

SEQ ID NO: 476 - DNA, 35 bp, synthetic construct
  source 1..35: mol_type = other DNA
cgacgataccattcgtggtatcgtcgttttttttt

SEQ ID NO: 477 - DNA, 35 bp, synthetic construct
  source 1..35: mol_type = other DNA
cgacgatgccattcgtggcatcgtcgttttgttgg

SEQ ID NO: 478 - DNA, 35 bp, synthetic construct
  source 1..35: mol_type = other DNA
cgacgataccattcgtggtatcgtcgttttgttgg

SEQ ID NO: 479 - DNA, 27 bp, synthetic construct
  source 1..27: mol_type = other DNA
cgcccgccgcagcgggcgtttgttgag

SEQ ID NO: 480 - DNA, 33 bp, synthetic construct
  source 1..33: mol_type = other DNA
gccggagcggcgcactgctccggcttttttgta

SEQ ID NO: 481 - DNA, 35 bp, synthetic construct
  source 1..35: mol_type = other DNA
tcggttacatgttcgcatgtaaccgattttgttgg

SEQ ID NO: 482 - DNA, 23 bp, synthetic construct
  source 1..23: mol_type = other DNA
cccgcttcggcgggttttgttgg

SEQ ID NO: 483 - DNA, 29 bp, synthetic construct
  source 1..29: mol_type = other DNA
cccgcatgttcgcatgcgggttttgttgg

SEQ ID NO: 484 - DNA, 29 bp, synthetic construct
  source 1..29: mol_type = other DNA
cgacgatattcgtatcgtcgttttttttt

SEQ ID NO: 485 - DNA, 31 bp, synthetic construct
  source 1..31: mol_type = other DNA
aggcctccccgcaggggggcctttttgcaac

SEQ ID NO: 486 - DNA, 30 bp, synthetic construct
  source 1..30: mol_type = other DNA
tgccgggagaacatcccggcattttgcaac

SEQ ID NO: 487 - DNA, 29 bp, synthetic construct
  source 1..29: mol_type = other DNA
tagcgtgccgcagcacgctattttgcaac

SEQ ID NO: 488 - DNA, 33 bp, synthetic construct
  source 1..33: mol_type = other DNA
ctaaagcgcccgcaggcgctttagtttgttgag

SEQ ID NO: 489 - DNA, 30 bp, synthetic construct
  source 1..30: mol_type = other DNA
gggcggtcgaacagatcgcccttttgcaac

SEQ ID NO: 490 - DNA, 34 bp, synthetic construct
  source 1..34: mol_type = other DNA
aggcctccctaaccacggggggcctttgttgtat

SEQ ID NO: 491 - DNA, 23 bp, synthetic construct
  source 1..23: mol_type = other DNA
cgacgttcgcgtcgttttttggg

SEQ ID NO: 492 - DNA, 39 bp, synthetic construct
  source 1..39: mol_type = other DNA
cctggtaagacgccgcagcgtcttatcaggttttttgta

SEQ ID NO: 493 - DNA, 35 bp, synthetic construct
  source 1..35: mol_type = other DNA
gatccagcccattcgtgggctggatcttaaaataa

SEQ ID NO: 494 - DNA, 33 bp, synthetic construct
  source 1..33: mol_type = other DNA
gccggagcggcgcactgctccggcttttgcaac

SEQ ID NO: 495 - DNA, 29 bp, synthetic construct
  source 1..29: mol_type = other DNA
tagcgtgccgcagcacgctatttgttgag

SEQ ID NO: 496 - DNA, 40 bp, synthetic construct
  source 1..40: mol_type = other DNA
cctggtaagacgcgaacagcgtcttatcaggttttgcaac

SEQ ID NO: 497 - DNA, 29 bp, synthetic construct
  source 1..29: mol_type = other DNA
cccgcatgttcgcatgcgggttatcaaaa

SEQ ID NO: 498 - DNA, 29 bp, synthetic construct
  source 1..29: mol_type = other DNA
cgacgatgttcgcatcgtcgttttgttgg

SEQ ID NO: 499 - DNA, 33 bp, synthetic construct
  source 1..33: mol_type = other DNA
gccggatcggcgcactgatccggcttttttgta

SEQ ID NO: 500 - DNA, 29 bp, synthetic construct
  source 1..29: mol_type = other DNA
tgccgggacgcatcccggcattttgcaac

SEQ ID NO: 501 - DNA, 34 bp, synthetic construct
  source 1..34: mol_type = other DNA
ctaaagcgccgaacaggcgctttagttgttgtat

SEQ ID NO: 502 - DNA, 40 bp, synthetic construct
  source 1..40: mol_type = other DNA
tagcgtgaccggtaaccactcggtcacgctattttttgta

SEQ ID NO: 503 - DNA, 23 bp, synthetic construct
  source 1..23: mol_type = other DNA
cgacgttcgcgtcgttttgttgg

SEQ ID NO: 504 - DNA, 32 bp, synthetic construct
  source 1..32: mol_type = other DNA
gggcggtctaaccacgatcgcccttgttgtat

SEQ ID NO: 505 - DNA, 29 bp, synthetic construct
  source 1..29: mol_type = other DNA
cgacgatattcgtatcgtcgttttgttgg

SEQ ID NO: 506 - DNA, 39 bp, synthetic construct
  source 1..39: mol_type = other DNA
cctggtaagacgccgcagcgtcgcatcaggttttttgta

SEQ ID NO: 507 - DNA, 30 bp, synthetic construct
  source 1..30: mol_type = other DNA
cgcccgctaaccacgcgggcgttgttgtat

SEQ ID NO: 508 - DNA, 39 bp, synthetic construct
  source 1..39: mol_type = other DNA
cctggtaagacgcagatgcgtcttatcaggttttttttt

SEQ ID NO: 509 - DNA, 37 bp, synthetic construct
  source 1..37: mol_type = other DNA
tagcgtgaccggcgcatcggtcacgctattttttgta

SEQ ID NO: 510 - DNA, 33 bp, synthetic construct
  source 1..33: mol_type = other DNA
gccggagcggcgcactgctccggctttgttgag

SEQ ID NO: 511 - DNA, 29 bp, synthetic construct
  source 1..29: mol_type = other DNA
tgccgggacgcatcccggcatttgttgag

SEQ ID NO: 512 - DNA, 32 bp, synthetic construct
  source 1..32: mol_type = other DNA
tgccgggataaccactcccggcattgttgtat

SEQ ID NO: 513 - DNA, 39 bp, synthetic construct
  source 1..39: mol_type = other DNA
cctggtaagacgccgcagcgtcgcatcaggttttgcaac

SEQ ID NO: 514 - DNA, 37 bp, synthetic construct
  source 1..37: mol_type = other DNA
tagcgtgaccggcgcatcggtcacgctatttgttgag

SEQ ID NO: 515 - DNA, 30 bp, synthetic construct
  source 1..30: mol_type = other DNA
tagcgtgcgaacagcacgctattttgcaac

SEQ ID NO: 516 - DNA, 37 bp, synthetic construct
  source 1..37: mol_type = other DNA
tagcgtgaccggcgcatcggtcacgctattttgcaac

SEQ ID NO: 517 - DNA, 34 bp, synthetic construct
  source 1..34: mol_type = other DNA
gccggagcgggaacactgctccggcttttgcaac

SEQ ID NO: 518 - DNA, 29 bp, synthetic construct
  source 1..29: mol_type = other DNA
cgacgatgttcgcatcgtcgttaaaataa

SEQ ID NO: 519 - DNA, 34 bp, synthetic construct
  source 1..34: mol_type = other DNA
gccggatcgggaacactgatccggcttgttgtat

SEQ ID NO: 520 - DNA, 30 bp, synthetic construct
  source 1..30: mol_type = other DNA
gggcggtcgaacagatcgcccttgttgtat

SEQ ID NO: 521 - DNA, 28 bp, synthetic construct
  source 1..28: mol_type = other DNA
cgcccgcgaacagcgggcgttgttgtat

SEQ ID NO: 522 - DNA, 35 bp, synthetic construct
  source 1..35: mol_type = other DNA
cgacgatgccattcgtggcatcgtcgttaaaataa

SEQ ID NO: 523 - DNA, 29 bp, synthetic construct
  source 1..29: mol_type = other DNA
cccgcatgttcgcatgcgggtttcaacaa

SEQ ID NO: 524 - DNA, 35 bp, synthetic construct
  source 1..35: mol_type = other DNA
tcggttaccgcttcggcggtaaccgattatcaaaa

SEQ ID NO: 525 - DNA, 39 bp, synthetic construct
  source 1..39: mol_type = other DNA
cctggtaagacgccgcagcgtcttatcaggttttgcaac

SEQ ID NO: 526 - DNA, 29 bp, synthetic construct
  source 1..29: mol_type = other DNA
gggcggtccgcagatcgccctttgttgag

SEQ ID NO: 527 - DNA, 42 bp, synthetic construct
  source 1..42: mol_type = other DNA
cctggtaagacgctaaccacgcgtcgcatcaggttttttgta

SEQ ID NO: 528 - DNA, 35 bp, synthetic construct
  source 1..35: mol_type = other DNA
cgacgatgccattcgtggcatcgtcgttatcaaaa

SEQ ID NO: 529 - DNA, 39 bp, synthetic construct
  source 1..39: mol_type = other DNA
cctggtaagacgccgcagcgtcgcatcaggtttgttgag

SEQ ID NO: 530 - DNA, 42 bp, synthetic construct
  source 1..42: mol_type = other DNA
cctggtaagacgctaaccacgcgtcttatcaggttgttgtat

SEQ ID NO: 531 - DNA, 23 bp, synthetic construct
  source 1..23: mol_type = other DNA
cgacgttcgcgtcgttatcaaaa

SEQ ID NO: 532 - DNA, 23 bp, synthetic construct
  source 1..23: mol_type = other DNA
cccgcttcggcgggttaaaataa

SEQ ID NO: 533 - DNA, 38 bp, synthetic construct
  source 1..38: mol_type = other DNA
tagcgtgaccgggaacatcggtcacgctattttgcaac

SEQ ID NO: 534 - DNA, 35 bp, synthetic construct
  source 1..35: mol_type = other DNA
cgacgataccattcgtggtatcgtcgttaaaataa

SEQ ID NO: 535 - DNA, 40 bp, synthetic construct
  source 1..40: mol_type = other DNA
cctggtaagacgcgaacagcgtcttatcaggttgttgtat

SEQ ID NO: 536 - DNA, 35 bp, synthetic construct
  source 1..35: mol_type = other DNA
cccgcatgccattcgtggcatgcgggtttcaacaa

SEQ ID NO: 537 - DNA, 39 bp, synthetic construct
  source 1..39: mol_type = other DNA
cctggtaagacgcagatgcgtcgcatcaggttttttttt

SEQ ID NO: 538 - DNA, 34 bp, synthetic construct
  source 1..34: mol_type = other DNA
gccggagcgggaacactgctccggcttgttgtat

SEQ ID NO: 539 - DNA, 33 bp, synthetic construct
  source 1..33: mol_type = other DNA
gccggatcggcgcactgatccggctttgttgag

SEQ ID NO: 540 - DNA, 35 bp, synthetic construct
  source 1..35: mol_type = other DNA
gatccagcccattcgtgggctggatcttatcaaaa

SEQ ID NO: 541 - DNA, 35 bp, synthetic construct
  source 1..35: mol_type = other DNA
cgacgatgccattcgtggcatcgtcgtttcaacaa

SEQ ID NO: 542 - DNA, 30 bp, synthetic construct
  source 1..30: mol_type = other DNA
tgccgggagaacatcccggcattgttgtat

SEQ ID NO: 543 - DNA, 33 bp, synthetic construct
  source 1..33: mol_type = other DNA
gccggatcggcgcactgatccggcttttgcaac

SEQ ID NO: 544 - DNA, 35 bp, synthetic construct
  source 1..35: mol_type = other DNA
tcggttaccgcttcggcggtaaccgatttcaacaa

SEQ ID NO: 545 - DNA, 29 bp, synthetic construct
  source 1..29: mol_type = other DNA
gatccagcttcggctggatcttatcaaaa

SEQ ID NO: 546 - DNA, 29 bp, synthetic construct
  source 1..29: mol_type = other DNA
gatccagcttcggctggatcttaaaataa

SEQ ID NO: 547 - DNA, 29 bp, synthetic construct
  source 1..29: mol_type = other DNA
gatccagcttcggctggatctttcaacaa

SEQ ID NO: 548 - DNA, 35 bp, synthetic construct
  source 1..35: mol_type = other DNA
tcggttacatgttcgcatgtaaccgattatcaaaa

SEQ ID NO: 549 - DNA, 23 bp, synthetic construct
  source 1..23: mol_type = other DNA
cgacgttcgcgtcgttttttttt

SEQ ID NO: 550 - DNA, 39 bp, synthetic construct
  source 1..39: mol_type = other DNA
cctggtaagacgccgcagcgtcttatcaggtttgttgag

SEQ ID NO: 551 - DNA, 30 bp, synthetic construct
  source 1..30: mol_type = other DNA
acggccctgaacaagggccgtttgttgtat

SEQ ID NO: 552 - DNA, 29 bp, synthetic construct
  source 1..29: mol_type = other DNA
cccgcatgttcgcatgcgggttaaaataa

SEQ ID NO: 553 - DNA, 29 bp, synthetic construct
  source 1..29: mol_type = other DNA
cgacgatattcgtatcgtcgttaaaataa

SEQ ID NO: 554 - DNA, 29 bp, synthetic construct
  source 1..29: mol_type = other DNA
cgacgatgttcgcatcgtcgttatcaaaa

SEQ ID NO: 555 - DNA, 23 bp, synthetic construct
  source 1..23: mol_type = other DNA
cgacgttcgcgtcgtttcaacaa

SEQ ID NO: 556 - DNA, 32 bp, synthetic construct
  source 1..32: mol_type = other DNA
aggcctcccgaacaggggggcctttgttgtat

SEQ ID NO: 557 - DNA, 35 bp, synthetic construct
  source 1..35: mol_type = other DNA
cgacgataccattcgtggtatcgtcgttatcaaaa

SEQ ID NO: 558 - DNA, 35 bp, synthetic construct
  source 1..35: mol_type = other DNA
cccgcatgccattcgtggcatgcgggttaaaataa

SEQ ID NO: 559 - DNA, 42 bp, synthetic construct
  source 1..42: mol_type = other DNA
cctggtaagacgctaaccacgcgtcttatcaggttttttgta

SEQ ID NO: 560 - DNA, 35 bp, synthetic construct
  source 1..35: mol_type = other DNA
tcggttacatgttcgcatgtaaccgattaaaataa

SEQ ID NO: 561 - DNA, 36 bp, synthetic construct
  source 1..36: mol_type = other DNA
gccggatcggtaaccacctgatccggcttgttgtat

SEQ ID NO: 562 - DNA, 35 bp, synthetic construct
  source 1..35: mol_type = other DNA
cccgcatgccattcgtggcatgcgggttatcaaaa

SEQ ID NO: 563 - DNA, 35 bp, synthetic construct
  source 1..35: mol_type = other DNA
tcggttaccgcttcggcggtaaccgattaaaataa

SEQ ID NO: 564 - DNA, 23 bp, synthetic construct
  source 1..23: mol_type = other DNA
cccgcttcggcgggttatcaaaa

SEQ ID NO: 565 - DNA, 23 bp, synthetic construct
  source 1..23: mol_type = other DNA
cccgcttcggcgggtttcaacaa

SEQ ID NO: 566 - DNA, 35 bp, synthetic construct
  source 1..35: mol_type = other DNA
tcggttacatgttcgcatgtaaccgatttcaacaa

SEQ ID NO: 567 - DNA, 29 bp, synthetic construct
  source 1..29: mol_type = other DNA
cgacgatattcgtatcgtcgttatcaaaa

SEQ ID NO: 568 - DNA, 32 bp, synthetic construct
  source 1..32: mol_type = other DNA
acggcccttaaccacagggccgtttgttgtat

SEQ ID NO: 569 - DNA, 40 bp, synthetic construct
  source 1..40: mol_type = other DNA
cctggtaagacgcgaacagcgtcgcatcaggttttgcaac

SEQ ID NO: 570 - DNA, 42 bp, synthetic construct
  source 1..42: mol_type = other DNA
cctggtaagacgctaaccacgcgtcgcatcaggttgttgtat

SEQ ID NO: 571 - DNA, 29 bp, synthetic construct
  source 1..29: mol_type = other DNA
cgacgatgttcgcatcgtcgtttcaacaa

SEQ ID NO: 572 - DNA, 35 bp, synthetic construct
  source 1..35: mol_type = other DNA
cgacgataccattcgtggtatcgtcgtttcaacaa

SEQ ID NO: 573 - DNA, 35 bp, synthetic construct
  source 1..35: mol_type = other DNA
gatccagcccattcgtgggctggatctttcaacaa

SEQ ID NO: 574 - DNA, 38 bp, synthetic construct
  source 1..38: mol_type = other DNA
tagcgtgaccgggaacatcggtcacgctattgttgtat

SEQ ID NO: 575 - DNA, 36 bp, synthetic construct
  source 1..36: mol_type = other DNA
gccggagcggtaaccacctgctccggcttgttgtat

SEQ ID NO: 576 - DNA, 40 bp, synthetic construct
  source 1..40: mol_type = other DNA
cctggtaagacgcgaacagcgtcgcatcaggttgttgtat

SEQ ID NO: 577 - DNA, 40 bp, synthetic construct
  source 1..40: mol_type = other DNA
tagcgtgaccggtaaccactcggtcacgctattgttgtat

SEQ ID NO: 578 - DNA, 29 bp, synthetic construct
  source 1..29: mol_type = other DNA
cgacgatattcgtatcgtcgtttcaacaa

SEQ ID NO: 579 - DNA, 32 bp, synthetic construct
  source 1..32: mol_type = other DNA
tagcgtgctaaccacgcacgctattgttgtat

SEQ ID NO: 580 - DNA, 36 bp, synthetic construct
  source 1..36: mol_type = other DNA
ctaaagcgcctaaccacggcgctttagttgttgtat

SEQ ID NO: 581 - DNA, 30 bp, synthetic construct
  source 1..30: mol_type = other DNA
tagcgtgcgaacagcacgctattgttgtat

SEQ ID NO: 582 - DNA, 30 bp, synthetic construct
  source 1..30: mol_type = other DNA
ggctagctcagtcctaggtacagtgctagc

SEQ ID NO: 583 - DNA, 30 bp, synthetic construct
  source 1..30: mol_type = other DNA
agctagctcagtcctaggtattatgctagc

SEQ ID NO: 584 - DNA, 30 bp, synthetic construct
  source 1..30: mol_type = other DNA
agctagctcagtcctaggtactgtgctagc

SEQ ID NO: 585 - DNA, 30 bp, synthetic construct
  source 1..30: mol_type = other DNA
agctagctcagtcctagggattatgctagc

SEQ ID NO: 586 - DNA, 30 bp, synthetic construct
  source 1..30: mol_type = other DNA
agctagctcagtcctaggtattgtgctagc

SEQ ID NO: 587 - DNA, 30 bp, synthetic construct
  source 1..30: mol_type = other DNA
ggctagctcagtcctaggtactatgctagc

SEQ ID NO: 588 - DNA, 30 bp, synthetic construct
  source 1..30: mol_type = other DNA
ggctagctcagtcctaggtatagtgctagc

SEQ ID NO: 589 - DNA, 30 bp, synthetic construct
  source 1..30: mol_type = other DNA
agctagctcagtcctaggtataatgctagc

SEQ ID NO: 590 - DNA, 30 bp, synthetic construct
  source 1..30: mol_type = other DNA
agctagctcagtcctagggactgtgctagc

SEQ ID NO: 591 - skipped

SEQ ID NO: 592 - DNA, 30 bp, synthetic construct
  source 1..30: mol_type = other DNA
ggctagctcagtcctaggtacaatgctagc

SEQ ID NO: 593 - DNA, 30 bp, synthetic construct; identical to SEQ ID NO: 588
  source 1..30: mol_type = other DNA

SEQ ID NO: 594 - DNA, 30 bp, synthetic construct; identical to SEQ ID NO: 585
  source 1..30: mol_type = other DNA

SEQ ID NO: 595 - DNA, 30 bp, synthetic construct
  source 1..30: mol_type = other DNA
ggctagctcagtcctagggattatgctagc

SEQ ID NO: 596 - DNA, 30 bp, synthetic construct; identical to SEQ ID NO: 592
  source 1..30: mol_type = other DNA

SEQ ID NO: 597 - DNA, 30 bp, synthetic construct
  source 1..30: mol_type = other DNA
agctagctcagcccttggtacaatgctagc

SEQ ID NO: 598 - DNA, 30 bp, synthetic construct
  source 1..30: mol_type = other DNA
agctagctcagtcctagggactatgctagc

SEQ ID NO: 599 - DNA, 30 bp, synthetic construct
  source 1..30: mol_type = other DNA
agctagctcagtcctagggattgtgctagc

SEQ ID NO: 600 - DNA, 30 bp, synthetic construct
  source 1..30: mol_type = other DNA
ggctagctcagtcctaggtattgtgctagc

SEQ ID NO: 601 - DNA, 30 bp, Escherichia coli
  source 1..30: mol_type = genomic DNA
aaagtgtgacgccgtgcaaataatcaatgt

SEQ ID NO: 602 - DNA, 30 bp, synthetic construct
  source 1..30: mol_type = other DNA
caccttcgggtgggcctttctgcgtttata

SEQ ID NO: 603 - DNA, 30 bp, synthetic construct; identical to SEQ ID NO: 582
  source 1..30: mol_type = other DNA

SEQ ID NO: 604 - DNA, 30 bp, synthetic construct; identical to SEQ ID NO: 583
  source 1..30: mol_type = other DNA

SEQ ID NO: 605 - DNA, 30 bp, synthetic construct; identical to SEQ ID NO: 584
  source 1..30: mol_type = other DNA

SEQ ID NO: 606 - DNA, 30 bp, synthetic construct; identical to SEQ ID NO: 585
  source 1..30: mol_type = other DNA

SEQ ID NO: 607 - DNA, 30 bp, synthetic construct; identical to SEQ ID NO: 588
  source 1..30: mol_type = other DNA

SEQ ID NO: 608 - DNA, 30 bp, synthetic construct; identical to SEQ ID NO: 595
  source 1..30: mol_type = other DNA

SEQ ID NO: 609 - DNA, 30 bp, synthetic construct; identical to SEQ ID NO: 592
  source 1..30: mol_type = other DNA

SEQ ID NO: 610 - DNA, 30 bp, synthetic construct; identical to SEQ ID NO: 599
  source 1..30: mol_type = other DNA

SEQ ID NO: 611 - DNA, 30 bp, synthetic construct; identical to SEQ ID NO: 600
  source 1..30: mol_type = other DNA

SEQ ID NO: 612 - DNA, 30 bp, synthetic construct
  source 1..30: mol_type = other DNA
gtttatacataggcgagtactctgttatgg

SEQ ID NO: 613 - DNA, 30 bp, synthetic construct
  source 1..30: mol_type = other DNA
agaggttccaactttcaccataatgaaaca

SEQ ID NO: 614 - DNA, 30 bp, synthetic construct
  source 1..30: mol_type = other DNA
taaacaactaacggacaattctacctaaca

SEQ ID NO: 615 - DNA, 30 bp, synthetic construct; identical to SEQ ID NO: 589
  source 1..30: mol_type = other DNA

SEQ ID NO: 616 - DNA, 30 bp, synthetic construct
  source 1..30: mol_type = other DNA
ggctagctcagtcctaggtattatgctagc

SEQ ID NO: 617 - DNA, 30 bp, synthetic construct; identical to SEQ ID NO: 587
  source 1..30: mol_type = other DNA

SEQ ID NO: 618 - DNA, 30 bp, synthetic construct
  source 1..30: mol_type = other DNA
gaggtaaaatagtcaacacgcacggtgtta

SEQ ID NO: 619 - DNA, 30 bp, synthetic construct
  source 1..30: mol_type = other DNA
caggccggaataactccctataatgcgcca

SEQ ID NO: 620 - DNA, 30 bp, synthetic construct; identical to SEQ ID NO: 592
  source 1..30: mol_type = other DNA

SEQ ID NO: 621-622 - skipped (2 entries)

SEQ ID NO: 623 - DNA, 30 bp, Escherichia coli
  source 1..30: mol_type = genomic DNA
gacgaatacttaaaatcgtcatacttattt

SEQ ID NO: 624 - DNA, 30 bp, Escherichia coli
  source 1..30: mol_type = genomic DNA
aaacctttcgcggtatggcatgatagcgcc

SEQ ID NO: 625 - DNA, 30 bp, Escherichia coli
  source 1..30: mol_type = genomic DNA
tgatagcgcccggaagagagtcaattcagg

SEQ ID NO: 626 - DNA, 30 bp, Escherichia coli
  source 1..30: mol_type = genomic DNA
ttatttaccgtgacgaactaattgctcgtg

SEQ ID NO: 627 - DNA, 30 bp, synthetic construct
  source 1..30: mol_type = other DNA
ttatgcttccggctcgtatgttgtgtggac

SEQ ID NO: 628 - DNA, 30 bp, synthetic construct
  source 1..30: mol_type = other DNA
ttatgcttccggctcgtatggtgtgtggac

SEQ ID NO: 629 - DNA, 30 bp, synthetic construct
  source 1..30: mol_type = other DNA
tgacaatatatatatatatataatgctagc

SEQ ID NO: 630 - DNA, 30 bp, synthetic construct
  source 1..30: mol_type = other DNA
acaatatatatatatatatataatgctagc

SEQ ID NO: 631 - DNA, 30 bp, synthetic construct
  source 1..30: mol_type = other DNA
aatatatatatatatatatataatgctagc

SEQ ID NO: 632 - DNA, 30 bp, synthetic construct
  source 1..30: mol_type = other DNA
tatatatatatatatatatataatgctagc

SEQ ID NO: 633 - DNA, 30 bp, synthetic construct; identical to SEQ ID NO: 632
  source 1..30: mol_type = other DNA

SEQ ID NO: 634 - DNA, 30 bp, synthetic construct
  source 1..30: mol_type = other DNA
aaaaaaaaaaaaaaaaaatataatgctagc

SEQ ID NO: 635 - DNA, 30 bp, synthetic construct; identical to SEQ ID NO: 634
  source 1..30: mol_type = other DNA

SEQ ID NO: 636 - DNA, 30 bp, synthetic construct
  source 1..30: mol_type = other DNA
ggaattgtgagcggataacaatttcacaca

SEQ ID NO: 637 - DNA, 30 bp, synthetic construct; identical to SEQ ID NO: 636
  source 1..30: mol_type = other DNA

SEQ ID NO: 638 - DNA, 30 bp, synthetic construct; identical to SEQ ID NO: 636
  source 1..30: mol_type = other DNA

SEQ ID NO: 639 - DNA, 30 bp, synthetic construct; identical to SEQ ID NO: 636
  source 1..30: mol_type = other DNA

SEQ ID NO: 640 - DNA, 30 bp, synthetic construct; identical to SEQ ID NO: 636
  source 1..30: mol_type = other DNA

SEQ ID NO: 641 - DNA, 30 bp, synthetic construct; identical to SEQ ID NO: 636
  source 1..30: mol_type = other DNA

SEQ ID NO: 642 - DNA, 30 bp, synthetic construct; identical to SEQ ID NO: 636
  source 1..30: mol_type = other DNA

SEQ ID NO: 643 - DNA, 30 bp, synthetic construct; identical to SEQ ID NO: 636
  source 1..30: mol_type = other DNA

SEQ ID NO: 644 - DNA, 30 bp, synthetic construct; identical to SEQ ID NO: 636
  source 1..30: mol_type = other DNA

SEQ ID NO: 645 - DNA, 30 bp, synthetic construct; identical to SEQ ID NO: 636
  source 1..30: mol_type = other DNA

SEQ ID NO: 646 - DNA, 30 bp, synthetic construct; identical to SEQ ID NO: 636
  source 1..30: mol_type = other DNA

SEQ ID NO: 647 - DNA, 30 bp, synthetic construct; identical to SEQ ID NO: 636
  source 1..30: mol_type = other DNA

SEQ ID NO: 648 - DNA, 30 bp, synthetic construct; identical to SEQ ID NO: 636
  source 1..30: mol_type = other DNA

SEQ ID NO: 649 - DNA, 30 bp, synthetic construct; identical to SEQ ID NO: 636
  source 1..30: mol_type = other DNA

SEQ ID NO: 650 - DNA, 30 bp, synthetic construct
  source 1..30: mol_type = other DNA
cattgtacctaggactgagctagccataaa

SEQ ID NO: 651 - DNA, 30 bp, synthetic construct
  source 1..30: mol_type = other DNA
ttgtgagcggataacaagatactgagcaca

SEQ ID NO: 652 - DNA, 30 bp, Inovirus M13
  source 1..30: mol_type = genomic DNA
cctgtttttatgttattctctctgtaaagg

SEQ ID NO: 653 - DNA, 30 bp, Inovirus M13
  source 1..30: mol_type = genomic DNA
aaatatttgcttatacaatcttcctgtttt

SEQ ID NO: 654 - DNA, 30 bp, Inovirus M13
  source 1..30: mol_type = genomic DNA
gctgataaaccgatacaattaaaggctcct

SEQ ID NO: 655 - DNA, 30 bp, Inovirus M13
  source 1..30: mol_type = genomic DNA
ctcttctcagcgtcttaatctaagctatcg

SEQ ID NO: 656 - DNA, 30 bp, Inovirus M13
  source 1..30: mol_type = genomic DNA
atgagccagttcttaaaatcgcataaggta

SEQ ID NO: 657 - DNA, 30 bp, Inovirus M13
  source 1..30: mol_type = genomic DNA
ctattgattgtgacaaaataaacttattcc

SEQ ID NO: 658 - DNA, 30 bp, Inovirus M13
  source 1..30: mol_type = genomic DNA
gtttcgcgcttggtataatcgctgggggtc

SEQ ID NO: 659 - DNA, 30 bp, Inovirus M13
  source 1..30: mol_type = genomic DNA
ctttgcttctgactataatagtcagggtaa

SEQ ID NO: 660 - DNA, 30 bp, synthetic construct
  source 1..30: mol_type = other DNA
aaaccgatacaattaaaggctcctgctagc

SEQ ID NO: 661 - DNA, 30 bp, synthetic construct
  source 1..30: mol_type = other DNA
caccacactgatagtgctagtgtagatcac

SEQ ID NO: 662 - DNA, 30 bp, synthetic construct
  source 1..30: mol_type = other DNA
gccggaataactccctataatgcgccacca

SEQ ID NO: 663 - DNA, 30 bp, Escherichia coli
  source 1..30: mol_type = genomic DNA
ttgacaagcttttcctcagctccgtaaact

SEQ ID NO: 664 - DNA, 30 bp, synthetic construct
  source 1..30: mol_type = other DNA
ggctagctcagccctaggtattatgctagc

SEQ ID NO: 665 - DNA, 30 bp, Escherichia coli
  source 1..30: mol_type = genomic DNA
catacgccgttatacgttgtttacgctttg

SEQ ID NO: 666 - DNA, 30 bp, synthetic construct
  source 1..30: mol_type = other DNA
atatatatatatatataatggaagcgtttt

SEQ ID NO: 667 - DNA, 30 bp, synthetic construct; identical to SEQ ID NO: 666
  source 1..30: mol_type = other DNA

SEQ ID NO: 668 - DNA, 30 bp, synthetic construct
  source 1..30: mol_type = other DNA
ccccgaaagcttaagaatataattgtaagc

SEQ ID NO: 669 - DNA, 30 bp, synthetic construct; identical to SEQ ID NO: 668
  source 1..30: mol_type = other DNA

SEQ ID NO: 670 - DNA, 30 bp, synthetic construct
  source 1..30: mol_type = other DNA
gattaaagaggagaaatactagagtactag

SEQ ID NO: 671 - DNA, 30 bp, synthetic construct; identical to SEQ ID NO: 602
  source 1..30: mol_type = other DNA

SEQ ID NO: 672 - DNA, 30 bp, synthetic construct; identical to SEQ ID NO: 602
  source 1..30: mol_type = other DNA

SEQ ID NO: 673 - DNA, 30 bp, synthetic construct; identical to SEQ ID NO: 602
  source 1..30: mol_type = other DNA

SEQ ID NO: 674 - DNA, 30 bp, synthetic construct; identical to SEQ ID NO: 582
  source 1..30: mol_type = other DNA

SEQ ID NO: 675 - DNA, 30 bp, synthetic construct
  source 1..30: mol_type = other DNA
tgctagctactagagattaaagaggagaaa

SEQ ID NO: 676 - DNA, 30 bp, Escherichia coli; identical to SEQ ID NO: 651
  source 1..30: mol_type = genomic DNA

SEQ ID NO: 677 - DNA, 30 bp, Escherichia coli; identical to SEQ ID NO: 651
  source 1..30: mol_type = genomic DNA

SEQ ID NO: 678 - DNA, 30 bp, Escherichia coli
  source 1..30: mol_type = genomic DNA
gtttctccatacccgtttttttgggctagc

SEQ ID NO: 679 - DNA, 30 bp, synthetic construct
  source 1..30: mol_type = other DNA
tgttatagtcgaatacctctggcggtgata

SEQ ID NO: 680 - DNA, 30 bp, synthetic construct
  source 1..30: mol_type = other DNA
attacaaactttcttgtatagatttaacgt

SEQ ID NO: 681 - DNA, 30 bp, synthetic construct
  source 1..30: mol_type = other DNA
atttataaatagtggtgatagatttaacgt

SEQ ID NO: 682 - DNA, 30 bp, synthetic construct
  source 1..30: mol_type = other DNA
tttcttgtatagatttacaatgtatcttgt

SEQ ID NO: 683 - DNA, 30 bp, synthetic construct
  source 1..30: mol_type = other DNA
tttcttgtagatacttacaatgtatcttgt

SEQ ID NO: 684 - DNA, 30 bp, synthetic construct
  source 1..30: mol_type = other DNA
ctttatgcttccggctcgtatgttgtgtgg

SEQ ID NO: 685 - DNA, 30 bp, synthetic construct
  source 1..30: mol_type = other DNA
ttttttgggctagcaagctttaccatggat

SEQ ID NO: 686 - DNA, 30 bp, Escherichia coli
  source 1..30: mol_type = genomic DNA
tgtttctccataccgtttttttgggctagc

SEQ ID NO: 687 - DNA, 30 bp, synthetic construct
  source 1..30: mol_type = other DNA
ttttggtacactccctatcagtgatagaga

SEQ ID NO: 688 - DNA, 30 bp, synthetic construct
  source 1..30: mol_type = other DNA
ctttttggtacactacctctggcggtgata

SEQ ID NO: 689 - DNA, 30 bp, synthetic construct
  source 1..30: mol_type = other DNA
tacgcaagaaaatggtttgttatagtcgaa

SEQ ID NO: 690 - DNA, 30 bp, synthetic construct
  source 1..30: mol_type = other DNA
gaaaaccttgtcaatgaagagcgatctatg

SEQ ID NO: 691 - DNA, 30 bp, synthetic construct
  source 1..30: mol_type = other DNA
ctcaaagcgggccagccgtagccgttacgc

SEQ ID NO: 692 - DNA, 30 bp, Escherichia coli
  source 1..30: mol_type = genomic DNA
ttctcgttcgactcatagctgaacacaaca

SEQ ID NO: 693 - DNA, 30 bp, synthetic construct
  source 1..30: mol_type = other DNA
gttctttaattatttaagtgttctttaatt

SEQ ID NO: 694 - DNA, 30 bp, synthetic construct
  source 1..30: mol_type = other DNA
cgtgcgtgttgataacaccgtgcgtgttga

SEQ ID NO: 695 - DNA, 30 bp, Escherichia coli
  source 1..30: mol_type = genomic DNA
gttacgtttatcgcggtgattgttacttat

SEQ ID NO: 696 - DNA, 30 bp, Escherichia coli
  source 1..30: mol_type = genomic DNA
gcaaaataaaatggaatgatgaaactgggt

SEQ ID NO: 697 - DNA, 30 bp, Escherichia coli; identical to SEQ ID NO: 695
  source 1..30: mol_type = genomic DNA

SEQ ID NO: 698 - DNA, 30 bp, Escherichia coli
  source 1..30: mol_type = genomic DNA
atttcacactgctattgagataattcacaa

SEQ ID NO: 699 - DNA, 30 bp, Staphylococcus aureus
  source 1..30: mol_type = genomic DNA
agattgtactaaatcgtataatgacagtga

SEQ ID NO: 700 - DNA, 30 bp, Peduovirus P2
  source 1..30: mol_type = genomic DNA
gacatctccggcgcaactgaaaataccact

SEQ ID NO: 701 - DNA, 30 bp, Peduovirus P2
  source 1..30: mol_type = genomic DNA
gaggatgcgcatcgtcgggaaactgatgcc

SEQ ID NO: 702 - DNA, 30 bp, Peduovirus P2
  source 1..30: mol_type = genomic DNA
catccgggactgatggcggaggatgcgcat

SEQ ID NO: 703 - DNA, 30 bp, Peduovirus P2
  source 1..30: mol_type = genomic DNA
aacttttatatattgtgcaatctcacatgc

SEQ ID NO: 704 - DNA, 30 bp, Peduovirus P2
  source 1..30: mol_type = genomic DNA
tgttgtccggtgtacgtcacaattttctta

SEQ ID NO: 705 - DNA, 30 bp, Peduovirus P2
  source 1..30: mol_type = genomic DNA
gtctgctgaaaatattcacaaaataaagcg

SEQ ID NO: 706 - DNA, 30 bp, synthetic construct
  source 1..30: mol_type = other DNA
gtgttgatgcttttatcaccgccagtggta

SEQ ID NO: 707 - DNA, 30 bp, synthetic construct
  source 1..30: mol_type = other DNA
agtgtgtggaattgtgagcggataacaatt

SEQ ID NO: 708 - DNA, 16 bp, Escherichia coli
  source 1..16: mol_type = genomic DNA
atgacaaaattgtcat

SEQ ID NO: 709 - DNA, 30 bp, Escherichia coli
  source 1..30: mol_type = genomic DNA
acatcttaaaagttttagtatcatattcgt

SEQ ID NO: 710 - DNA, 30 bp, synthetic construct
  source 1..30: mol_type = other DNA
ctgaaagcgcataccgctatggagggggtt

SEQ ID NO: 711 - DNA, 30 bp, synthetic construct; identical to SEQ ID NO: 710
  source 1..30: mol_type = other DNA

SEQ ID NO: 712 - DNA, 30 bp, synthetic construct
  source 1..30: mol_type = other DNA
aacgaatataacaggtgggagatgagagga

SEQ ID NO: 713 - DNA, 30 bp, Escherichia coli
  source 1..30: mol_type = genomic DNA
aatatttcctcattttccacagtgaagtga

SEQ ID NO: 714 - DNA, 30 bp, synthetic construct; identical to SEQ ID NO: 689
  source 1..30: mol_type = other DNA

SEQ ID NO: 715 - DNA, 30 bp, Vibrio cholerae
  source 1..30: mol_type = genomic DNA
atttaattgttttgatcaattatttttctg

SEQ ID NO: 716 - DNA, 30 bp, synthetic construct
  source 1..30: mol_type = other DNA
tcttgcgtaaacctgtacgatcctacaggt

SEQ ID NO: 717 - DNA, 30 bp, synthetic construct
  source 1..30: mol_type = other DNA
attattctgcatttttggggagaatggact

SEQ ID NO: 718 - DNA, 30 bp, Escherichia coli
  source 1..30: mol_type = genomic DNA
ccttgctggaaggtttaacctttatcacag

SEQ ID NO: 719 - DNA, 19 bp, synthetic construct
  source 1..19: mol_type = other DNA
atgatgtgtccatggatta

SEQ ID NO: 720 - DNA, 26 bp, synthetic construct
  source 1..26: mol_type = other DNA
atgatagacgatgtgcggacaacgtg

SEQ ID NO: 721 - DNA, 30 bp, Escherichia coli
  source 1..30: mol_type = genomic DNA
cattagccgccaccatggggttaagtagca

SEQ ID NO: 722 - DNA, 30 bp, synthetic construct; identical to SEQ ID NO: 681
  source 1..30: mol_type = other DNA

SEQ ID NO: 723 - DNA, 30 bp, synthetic construct
  source 1..30: mol_type = other DNA
ataaagccatcacgagtaccatagaggatc

SEQ ID NO: 724 - DNA, 30 bp, synthetic construct
  source 1..30: mol_type = other DNA
tttgtcttttcttgcttaataatgttgtca

SEQ ID NO: 725 - DNA, 30 bp, synthetic construct; identical to SEQ ID NO: 724
  source 1..30: mol_type = other DNA

SEQ ID NO: 726 - DNA, 30 bp, Pseudomonas aeruginosa
  source 1..30: mol_type = genomic DNA
atcctcctttagtcttccccctcatgtgtg

SEQ ID NO: 727 - DNA, 30 bp, Pseudomonas aeruginosa
  source 1..30: mol_type = genomic DNA
taaaattatgaaatttgcataaattcttca

SEQ ID NO: 728 - DNA, 30 bp, Pseudomonas aeruginosa
  source 1..30: mol_type = genomic DNA
gaaatctggcagtttttggtacacgaaagc

SEQ ID NO: 729 - DNA, 30 bp, Pseudomonas aeruginosa
  source 1..30: mol_type = genomic DNA
tgccagttctggcaggtctaaaaagtgttc

SEQ ID NO: 730 - DNA, 30 bp, Bacillus subtilis
  source 1..30: mol_type = genomic DNA
cacagaacttgcatttatataaagggaaag

SEQ ID NO: 731 - DNA, 30 bp, synthetic construct
  source 1..30: mol_type = other DNA
acaccgtgcgtgttgatatagtcgaataaa

SEQ ID NO: 732 - DNA, 30 bp, synthetic construct
  source 1..30: mol_type = other DNA
aaaattatgaaatttgtataaattcttcag

SEQ ID NO: 733 - DNA, 30 bp, synthetic construct
  source 1..30: mol_type = other DNA
ggttctttttggtacctctggcggtgataa

SEQ ID NO: 734 - DNA, 30 bp, synthetic construct
  source 1..30: mol_type = other DNA
tgtaggatcgtacaggtataaattcttcag

SEQ ID NO: 735 - DNA, 30 bp, synthetic construct
  source 1..30: mol_type = other DNA
caagaaaatggtttgttatagtcgaataaa

SEQ ID NO: 736 - DNA, 30 bp, synthetic construct
  source 1..30: mol_type = other DNA
ctatctcatttgctagtatagtcgaataaa

SEQ ID NO: 737 - DNA, 30 bp, Escherichia coli; identical to SEQ ID NO: 695
  source 1..30: mol_type = genomic DNA

SEQ ID NO: 738 - DNA, 30 bp, synthetic construct; identical to SEQ ID NO: 695
  source 1..30: mol_type = other DNA

SEQ ID NO: 739 - DNA, 30 bp, synthetic construct; identical to SEQ ID NO: 695
  source 1..30: mol_type = other DNA

SEQ ID NO: 740 - DNA, 30 bp, Escherichia coli
  source 1..30: mol_type = genomic DNA
ataaatgcttgactctgtagcgggaaggcg

SEQ ID NO: 741 - DNA, 30 bp, Escherichia coli
  source 1..30: mol_type = genomic DNA
aaaactggtagtaggactggagattggtac

SEQ ID NO: 742 - DNA, 30 bp, synthetic construct
  source 1..30: mol_type = other DNA
gggacacaaacatcaagaggatatgagatt

SEQ ID NO: 743 - DNA, 30 bp, Escherichia coli
  source 1..30: mol_type = genomic DNA
gtcaaaatgaccgaaacgggtggtaacttc

SEQ ID NO: 744 - DNA, 30 bp, Escherichia coli
  source 1..30: mol_type = genomic DNA
agtaatcttatcgccagtttggtctggtca

SEQ ID NO: 745 - DNA, 30 bp, Escherichia coli; identical to SEQ ID NO: 744
  source 1..30: mol_type = genomic DNA

SEQ ID NO: 746 - DNA, 30 bp, Escherichia coli
  source 1..30: mol_type = genomic DNA
aattctgaacaacatccgtactcttcgtgc

SEQ ID NO: 747 - DNA, 30 bp, Escherichia coli
  source 1..30: mol_type = genomic DNA
tcgataagattaccgatcttacctgaagct

SEQ ID NO: 748 - DNA, 30 bp, Escherichia coli
  source 1..30: mol_type = genomic DNA
cgatctattcacctgaaagagaaataaaaa

SEQ ID NO: 749 - DNA, 30 bp, Escherichia coli
  source 1..30: mol_type = genomic DNA
attaatgatcgcaacctatttattacaaca

SEQ ID NO: 750 - DNA, 30 bp, Escherichia coli
  source 1..30: mol_type = genomic DNA
aaacgttagtttgaatggaaagatgcctgc

SEQ ID NO: 751 - DNA, 30 bp, Lambdavirus lambda
  source 1..30: mol_type = genomic DNA
tttgcacgaaccatatgtaagtatttcctt

SEQ ID NO: 752 - DNA, 30 bp, Canis lupus
  source 1..30: mol_type = genomic DNA
taacacttatttaattaaaaagaggagaaa

SEQ ID NO: 753 - DNA, 30 bp, Escherichia coli
  source 1..30: mol_type = genomic DNA
tagaaacaaaatgtaacatctctatggaca

SEQ ID NO: 754 - DNA, 30 bp, synthetic construct
  source 1..30: mol_type = other DNA
acaggaaacagctatgaccatgattacgcc

SEQ ID NO: 755 - DNA, 30 bp, Escherichia coli
  source 1..30: mol_type = genomic DNA
agcgacgtctgatgacgtaatttctgcctc

SEQ ID NO: 756 - DNA, 30 bp, Escherichia coli
  source 1..30: mol_type = genomic DNA
gttcactctataccgctgaaggtgtaatgg

SEQ ID NO: 757 - DNA, 30 bp, synthetic construct
  source 1..30: mol_type = other DNA
tagtttataatttaagtgttctttaatttc

SEQ ID NO: 758 - DNA, 30 bp, synthetic construct; identical to SEQ ID NO: 602
  source 1..30: mol_type = other DNA

SEQ ID NO: 759 - DNA, 30 bp, synthetic construct; identical to SEQ ID NO: 602
  source 1..30: mol_type = other DNA

SEQ ID NO: 760 - DNA, 30 bp, synthetic construct; identical to SEQ ID NO: 602
  source 1..30: mol_type = other DNA

SEQ ID NO: 761 - DNA, 30 bp, synthetic construct; identical to SEQ ID NO: 602
  source 1..30: mol_type = other DNA

SEQ ID NO: 762 - DNA, 30 bp, synthetic construct
  source 1..30: mol_type = other DNA
cgagcacttcaccaacaaggaccatagcat

SEQ ID NO: 763 - DNA, 30 bp, synthetic construct; identical to SEQ ID NO: 602
  source 1..30: mol_type = other DNA

SEQ ID NO: 764 - DNA, 30 bp, synthetic construct
  source 1..30: mol_type = other DNA
catggcatggatgaactatacaaataataa

SEQ ID NO: 765 - DNA, 30 bp, synthetic construct; identical to SEQ ID NO: 602
  source 1..30: mol_type = other DNA

SEQ ID NO: 766 - DNA, 30 bp, synthetic construct; identical to SEQ ID NO: 602
  source 1..30: mol_type = other DNA

SEQ ID NO: 767 - DNA, 30 bp, synthetic construct; identical to SEQ ID NO: 602
  source 1..30: mol_type = other DNA

SEQ ID NO: 768 - DNA, 30 bp, synthetic construct; identical to SEQ ID NO: 602
  source 1..30: mol_type = other DNA

SEQ ID NO: 769 - DNA, 30 bp, synthetic construct; identical to SEQ ID NO: 602
  source 1..30: mol_type = other DNA

SEQ ID NO: 770 - DNA, 30 bp, synthetic construct; identical to SEQ ID NO: 602
  source 1..30: mol_type = other DNA

SEQ ID NO: 771 - DNA, 30 bp, synthetic construct; identical to SEQ ID NO: 602
  source 1..30: mol_type = other DNA

SEQ ID NO: 772 - DNA, 30 bp, synthetic construct; identical to SEQ ID NO: 602
  source 1..30: mol_type = other DNA

SEQ ID NO: 773 - DNA, 30 bp, synthetic construct; identical to SEQ ID NO: 602
  source 1..30: mol_type = other DNA

SEQ ID NO: 774 - DNA, 30 bp, synthetic construct; identical to SEQ ID NO: 602
  source 1..30: mol_type = other DNA

SEQ ID NO: 775 - DNA, 30 bp, synthetic construct; identical to SEQ ID NO: 602
  source 1..30: mol_type = other DNA

SEQ ID NO: 776 - DNA, 30 bp, synthetic construct; identical to SEQ ID NO: 602
  source 1..30: mol_type = other DNA

SEQ ID NO: 777 - DNA, 30 bp, synthetic construct; identical to SEQ ID NO: 602
  source 1..30: mol_type = other DNA

SEQ ID NO: 778 - DNA, 30 bp, synthetic construct; identical to SEQ ID NO: 602
  source 1..30: mol_type = other DNA

SEQ ID NO: 779 - DNA, 30 bp, synthetic construct; identical to SEQ ID NO: 602
  source 1..30: mol_type = other DNA

SEQ ID NO: 780 - DNA, 30 bp, synthetic construct; identical to SEQ ID NO: 686
  source 1..30: mol_type = other DNA

SEQ ID NO: 781 - DNA, 30 bp, synthetic construct; identical to SEQ ID NO: 686
  source 1..30: mol_type = other DNA

SEQ ID NO: 782 - DNA, 30 bp, synthetic construct
  source 1..30: mol_type = other DNA
caagaaaatggtttgttactttcgaataaa

SEQ ID NO: 783 - DNA, 30 bp, Escherichia coli
  source 1..30: mol_type = genomic DNA
ttttatcgcaactctctactgtttctccat

SEQ ID NO: 784 - DNA, 30 bp, synthetic construct
  source 1..30: mol_type = other DNA
gtttctccattactagagaaagaggggaca

SEQ ID NO: 785 - DNA, 30 bp, synthetic construct; identical to SEQ ID NO: 602
  source 1..30: mol_type = other DNA

SEQ ID NO: 786 - DNA, 30 bp, synthetic construct
  source 1..30: mol_type = other DNA
aataactctgatagtgctagtgtagatctc

SEQ ID NO: 787 - DNA, 30 bp, synthetic construct; identical to SEQ ID NO: 602
  source 1..30: mol_type = other DNA

SEQ ID NO: 788 - DNA, 30 bp, synthetic construct; identical to SEQ ID NO: 602
  source 1..30: mol_type = other DNA

SEQ ID NO: 789 - DNA, 30 bp, synthetic construct; identical to SEQ ID NO: 735
  source 1..30: mol_type = other DNA

SEQ ID NO: 790 - DNA, 30 bp, synthetic construct; identical to SEQ ID NO: 735
  source 1..30: mol_type = other DNA

SEQ ID NO: 791 - DNA, 30 bp, synthetic construct; identical to SEQ ID NO: 735
  source 1..30: mol_type = other DNA

SEQ ID NO: 792 - DNA, 30 bp, synthetic construct
  source 1..30: mol_type = other DNA
taatgtgtggaattgtgagcgctcacaatt

SEQ ID NO: 793 - DNA, 30 bp, synthetic construct
  source 1..30: mol_type = other DNA
tactgtgtggaattgtgagcgctcacaatt

SEQ ID NO: 794 - DNA, 30 bp, synthetic construct; identical to SEQ ID NO: 792
  source 1..30: mol_type = other DNA

SEQ ID NO: 795 - DNA, 30 bp, synthetic construct; identical to SEQ ID NO: 793
  source 1..30: mol_type = other DNA

SEQ ID NO: 796 - DNA, 30 bp, synthetic construct
  source 1..30: mol_type = other DNA
tgctttccacgaacttgaaaacgctggagg

SEQ ID NO: 797 - DNA, 30 bp, Muvirus mu
  source 1..30: mol_type = genomic DNA
tcctcaatatcctgtgatgaataaccgtac

SEQ ID NO: 798 - DNA, 30 bp, Muvirus mu
  source 1..30: mol_type = genomic DNA
tttttaagatagtggcgaattgatgcaaag

SEQ ID NO: 799 - DNA, 30 bp, synthetic construct; identical to SEQ ID NO: 735
  source 1..30: mol_type = other DNA

SEQ ID NO: 800 - DNA, 30 bp, synthetic construct; identical to SEQ ID NO: 735
  source 1..30: mol_type = other DNA

SEQ ID NO: 801 - DNA, 30 bp, synthetic construct; identical to SEQ ID NO: 735
  source 1..30: mol_type = other DNA

SEQ ID NO: 802 - DNA, 30 bp, Escherichia coli
  source 1..30: mol_type = genomic DNA
ctctactgtttctccatacccgtttttttg

SEQ ID NO: 803 - DNA, 30 bp, Escherichia coli
  source 1..30: mol_type = genomic DNA
tgtttctccatacccgttttttgggctaac

SEQ ID NO: 804 - DNA, 30 bp, synthetic construct
  source 1..30: mol_type = other DNA
aatgtgtggaattgtgagcggataacaatt

SEQ ID NO: 805 - DNA, 30 bp, synthetic construct; identical to SEQ ID NO: 735
  source 1..30: mol_type = other DNA

SEQ ID NO: 806 - DNA, 30 bp, synthetic construct
  source 1..30: mol_type = other DNA
gtgttgactattttacctctggcggtgata

SEQ ID NO: 807 - DNA, 30 bp, Pseudomonas aeruginosa
  source 1..30: mol_type = genomic DNA
gttagctttcgaattggctaaaaagtgttc

SEQ ID NO: 808 - DNA, 30 bp, Pseudomonas aeruginosa
  source 1..30: mol_type = genomic DNA
ccattctgctttccacgaacttgaaaacgc

SEQ ID NO: 809 - DNA, 30 bp, Pseudomonas aeruginosa
  source 1..30: mol_type = genomic DNA
ggccgcgggttctttttggtacacgaaagc

SEQ ID NO: 810 - DNA, 30 bp, Escherichia coli; identical to SEQ ID NO: 783
  source 1..30: mol_type = genomic DNA

SEQ ID NO: 811 - DNA, 30 bp, Escherichia coli; identical to SEQ ID NO: 717
  source 1..30: mol_type = genomic DNA

SEQ ID NO: 812 - DNA, 30 bp, Escherichia coli; identical to SEQ ID NO: 717
  source 1..30: mol_type = genomic DNA

SEQ ID NO: 813 - DNA, 30 bp, Escherichia coli
  source 1..30: mol_type = genomic DNA
aacgttagtttgaatggaaagatgcctgca

SEQ ID NO: 814 - DNA, 30 bp, synthetic construct
  source 1..30: mol_type = other DNA
aagaaaatggtttgttgatactcgaataaa

SEQ ID NO: 815 - DNA, 119 bp, synthetic construct
  source 1..119: mol_type = other DNA
ctgtcgggtttcgccacctctgacttgagcgtcgatttttgtgatgctcgtcaggggggcggagcctatggaaaaacgccagcaacgcgatcccgcgaaattaatacgactcactatag

SEQ ID NO: 816 - DNA, 232 bp, synthetic construct
  source 1..232: mol_type = other DNA
ctgtcgggtttcgccacctctgacttgagcgtcgatttttgtgatgctcgtcaggggggcggagcctatggaaaccggcttatcggtcagtttcacctgatttacgtaaaaacccgcttcggcgggtttttgcttttggaggggcagaaagatgaatgactgtccacgacgctatacccaaaagaaaaacgccagcaacgcgatcccgcgaaattaatacgactcactatag

SEQ ID NO: 817 - DNA, 282 bp, synthetic construct
  source 1..282: mol_type = other DNA
ctgtcgggtttcgccacctctgacttgagcgtcgatttttgtgatgctcgtcaggggggcggagcctatggaaaaacgccagcaacgcggcctttttacggttcctggccttttccggcttatcggtcagtttcacctgatttacgtaaaaacccgcttcggcgggtttttgcttttggaggggcagaaagatgaatgactgtccacgacgctatacccaaaagaaagctggccttttgctcacatgttcttatcccgcgaaattaatacgactcactatag

SEQ ID NO: 818 - DNA, 226 bp, synthetic construct
  source 1..226: mol_type = other DNA
gggcggagcctatggaaaaacgccagcaacgcggcctttttacggttcctggccttttccggcttatcggtcagtttcacctgatttacgtaaaaacccgcttcggcgggtttttgcttttggaggggcagaaagatgaatgactgtccacgacgctatacccaaaagaaagctggccttttgctcacatgttcttatcccgcgaaattaatacgactcactatag

SEQ ID NO: 819 - DNA, 18 bp, Enterobacteria phage T7
  source 1..18: mol_type = genomic DNA
taatacgactcactatag

SEQ ID NO: 820 - DNA, 113 bp, Escherichia coli
  source 1..113: mol_type = genomic DNA
ccggcttatcggtcagtttcacctgatttacgtaaaaacccgcttcggcgggtttttgcttttggaggggcagaaagatgaatgactgtccacgacgctatacccaaaagaaa

SEQ ID NO: 821 - DNA, 29 bp, synthetic construct
  source 1..29: mol_type = other DNA
gggcggtccgcagatcgcccttttttgta

(19) United States
(12) Patent Application Publication
(10) Pub. No.: US 2026/0234646 A1
(43) Pub. Date: Aug. 13, 2026

(54) CELL-FREE EXPRESSION VECTORS AND METHODS FOR IMPROVED PROTEIN PRODUCTION

(71) Applicant: National Resilience, Inc., San Diego, CA (US)

(72) Inventors: Ryan Rezvani, San Diego, CA (US); Weston Kightlinger, San Diego, CA (US); Andrew Hunt, San Diego, CA (US); Alex Javanpour, San Diego, CA (US)

(21) Appl. No.: 19/155,910

(22) PCT Filed: Feb. 13, 2024
(86) PCT No.: PCT/US2024/015581

§ 371 Date: Aug. 12, 2025

Related U.S. Application Data
(60) Provisional application No. 63/445,245, filed on Feb. 13, 2023.

(51) Int. Cl. C12N 15/73; C07K 14/435

(52) U.S. Cl. CPC C12N 15/73; C07K 14/43595; C12N 2830/36; C12N 2830/40

(57) ABSTRACT

The present disclosure relates to improved methods, compositions, and kits for cell-free expression of proteins in vitro.

### CLAIM OF PRIORITY

This application claims the benefit of U.S. Provisional Application Ser. No. 63/445,245, filed on Feb. 13, 2023. The entire contents of the foregoing are incorporated herein by reference.

### TECHNICAL FIELD

The present invention relates to cell-free systems, kits, and methods for producing proteins.

### BACKGROUND

Cell-free protein-synthesis (CFPS) systems are emerging as an attractive alternative to conventional expression systems that rely on living cells (Katzen et al., “The Past, Present and Future of Cell-Free Protein Synthesis,” Trends Biotechnol. 23: 150-156 (2005)). This is because, over the past decade, cell-free protein synthesis reactions: (i) can be completed in less than a day; (ii) use cheaper reagents; (iii) fold complex proteins by routinely forming disulfide bonds; and (iv) can be scaled to 100 L. Two main approaches have been used for in vitro transcription/translation: one is based on cell-free extracts (CFEs), often derived from Escherichia coli, rabbit reticulocytes, or wheat germ, and the second is based on reconstituted protein synthesis from purified components (Shimizu et al., “Cell-Free Translation Reconstituted With Purified Components,” Nat. Biotechnol. 19:751-755 (2001)). Because of their ability to co-activate multiple biochemical networks in a single integrated platform (Jewett et al., “An Integrated Cell-Free Metabolic Platform for Protein Production and Synthetic Biology,” Mol. Syst. Biol. 4:220 (2008)), CFPS systems are increasingly used in many important biotechnology and synthetic biology applications (Ryabova et al., “Functional Antibody Production Using Cell-Free Translation: Effects of Protein Disulfide Isomerase and Chaperones,” Nat. Biotechnol. 15:79-84 (1997); Noireaux et al., “Principles of Cell-Free Genetic Circuit Assembly,” Proc. Nat'l. Acad. Sci. U.S.A. 100:12672-12677 (2003); Yang et al., “Rapid Expression of Vaccine Proteins for B-Cell Lymphoma in a Cell-Free System,” Biotechnol. Bioeng. 89:503-51.1 (2005)).

### SUMMARY

The present disclosure relates to improved cell-free systems, methods, and kits for expressing proteins in vitro. In particular, the present disclosure relates to avoiding, reducing, or preventing read-through of toxic product proteins during generation of plasmids, while avoiding, decreasing, or preventing a reduction in protein synthesis of the product protein during CFPS at the commercial scale.

In one aspect, the disclosure provides methods of creating or modifying a cell-free expression vector, the methods including obtaining a cell-free expression vector including (i) an origin of replication (ori), (ii) a nucleic acid sequence encoding a protein or RNA, (iii) a promoter arranged to drive expression of the protein or RNA, and (iv) one or more selectable markers; and inserting into the cell-free expression vector an insulating terminator sequence at a location that is between 0 and 10,000 nucleotides in a 5′ direction from the promoter.

In another aspect, the disclosure provides methods of performing protein or RNA synthesis in vitro, the methods including synthesizing protein or RNA in vitro using a cell-free expression vector, wherein the cell-free expression vector includes (i) an origin of replication (ori); (ii) a nucleic acid encoding protein or RNA to be synthesized; (iii) a promoter arranged to drive expression of the protein or RNA; (iv) an insulating terminator sequence located between 0 and 10,000 nucleotides in a 5′ direction from the promoter; and (iv) one or more selectable markers.

These methods can avoid or reduce read-through of toxic product proteins during plasmid generation, while avoiding or decreasing a reduction in protein synthesis of the product protein during cell-free protein synthesis. For example, the methods can avoid or reduce production of toxic RNA products.

In general, the cell-free expression vectors with the insulating terminators enable synthesis of the protein or RNA at higher yields, with higher growth rates, and/or with fewer sequence mutations than synthesis of the protein or RNA using a cell-free expression vector without an insulating terminator. In some instances, the cell-free expression vector with the insulating terminator enables synthesis of the protein or RNA at higher levels compared to levels of synthesis of the protein or RNA using a cell-free expression vector without an insulating terminator.

In certain embodiments of the new methods, the cell-free expression vector contains a gene for a protein or RNA that inhibits or slows cellular replication in cells containing the cell-free expression vector and/or a gene for a protein or RNA that reduces plasmid yield from the cells containing the cell-free expression vector.

In various embodiments, the insulating terminator sequence can be mpB-T1 (SEQ ID NO: 820), rmB T1 (SEQ ID NO: 14), L3S2P21 (SEQ ID NO: 318), or L3S2P56 (SEQ ID NO: 319). In various embodiments, the promoter can be a T7 phage promoter, a lac promoter, a trp promoter, a recA promoter, a ribosomal RNA promoter, a Sp6 promoter, an araBad promoter, a pTac promoter, or a J23119 promoter.

In some embodiments, the insulating terminator sequence is located 27 to 37 or 35 to 37, or 37 nucleotides in the 5′ direction from the promoter. In some embodiments, the insulating terminator sequence is located about 10, about 15, about 20, about 25, about 30, about 35, about 40, about 45, about 50, about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10,000 nucleotides in the 5′ direction from the promoter that is arranged to drive expression of the protein or RNA.

In certain embodiments, the insulating terminator sequence is located 0 to 10,000 nucleotides in a 3′ direction of the ori, e.g., the insulating terminator sequence is located 30 to 40 nucleotides, or 40 nucleotides, in the 3′ direction of the ori. In some embodiments, the insulating terminator sequence is located about 10, about 15, about 20, about 25, about 30, about 35, about 40, about 45, about 50, about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10,000 nucleotides in the 3′ direction of the ori.

In some embodiments, the synthesizing step includes using a cell-free protein synthesis platform, for example, wherein the cell-free protein synthesis platform includes a system for in vitro transcription of mRNA and/or translation of polypeptides.

In certain embodiments, the cell-free expression vector further includes a Ribosome-binding site (RBS) and/or an Open Reading Frame (ORF).

In another aspect, the disclosure provides kits for use in a method of modifying a cell-free expression vector, wherein the kits include a cell-free expression vector that includes (i) an origin of replication (ori), (ii) a nucleic acid sequence encoding a protein or RNA, (iii) a promoter arranged to drive expression of the protein or RNA, and (iv) one or more selectable markers; an insulating terminator sequence that is to be inserted at a location between 0 and 10,000 nucleotides in a 5′ direction from the promoter in the cell-free expression vector; and cloning reagents.

In another aspect, the disclosure provides kits for performing protein or RNA synthesis in vitro, where the kits include reagents for cell-free protein or RNA synthesis; and a cell-free expression vector comprising (i) an origin of replication (ori); (ii) a nucleic acid encoding protein or RNA to be synthesized; (iii) a promoter arranged to drive expression of the protein or RNA; (iv) an insulating terminator sequence that is located between 0 and 10,000 nucleotides in a 5′ direction from the promoter; and (iv) one or more selectable markers.

In these kits, the insulating terminator sequence can be mpB-T1 (SEQ ID NO: 820), rrnB T1 (SEQ ID NO: 14), L3S2P21 (SEQ ID NO: 318), or L3S2P56 (SEQ ID NO: 319), and/or the promoter can be a T7 phage promoter, a lac promoter, a trp promoter, a recA promoter, a ribosomal RNA promoter, a Sp6 promoter, an araBad promoter, a pTac promoter, or a J23119 promoter.

In embodiments of these kits, the insulating terminator can be located between 0 and 10,000 nucleotides in a 5′ direction from the promoter, e.g., 27 to 37, or 37, nucleotides in the 5′ direction from the promoter. In some embodiments, the insulating terminator sequence is located about 10, about 15, about 20, about 25, about 30, about 35, about 40, about 45, about 50, about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10,000 nucleotides in the 5′ direction from the promoter.

In some embodiments of the kits, the insulating terminator sequence is located 0 to 10,000 nucleotides, or 30 to 40 nucleotides, or 40 nucleotides, in a 3′ direction of the ori. In some embodiments, the insulating terminator sequence is located about 10, about 15, about 20, about 25, about 30, about 35, about 40, about 45, about 50, about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10,000 nucleotides in the 3′ direction of the ori.

In certain embodiments of the kits, the cell-free expression vector further includes a Ribosome-binding site (RBS) and/or an Open Reading Frame (ORF).

Also provided herein are cell-free expression vectors including (i) an origin of replication (ori), (ii) a nucleic acid sequence encoding a protein or RNA, (iii) a promoter arranged to drive expression of the protein or RNA, (iv) one or more selectable markers, and (v) an insulating terminator sequence at a location that is between 0 and 10,000 nucleotides in a 5′ direction from the promoter.

In these cell-free expression vectors, the insulating terminator sequence is mpB-T1 (SEQ ID NO: 820), rmB T1 (SEQ ID NO: 14), L3S2P21 (SEQ ID NO: 318), or L3S2P56 (SEQ ID NO: 319), and/or the promoter can be a T7 phage promoter, a lac promoter, a trp promoter, a recA promoter, a ribosomal RNA promoter, a Sp6 promoter, an araBad promoter, a pTac promoter, or a J23119 promoter.

In some embodiments of the cell-free expression vectors, the cell-free expression vectors includes a backbone. In some instances, the backbone is from one of the following vectors: pJL1, pY71, p70a, pBEST, pEXP5, or pT7CFE.

In certain embodiments of the cell-free expression vectors, at least a portion of the cell-free expression vector includes the following sequence: gggcggagcctatggaaaaacgccagcaacgcggcctttttacggttcctggccttttccggcttatcggtcagtttcacctg atttacgtaaaaacccgcttcggcgggtttttgcttttggaggggcagaaagatgaatgactgtccacgacgctatacccaaa agaaagctggccttttgctcacatgttcttatcccgcgaaattaatacgactcactatag (SEQ ID NO: 818).

In some aspects of the cell-free expression vectors, the vectors include, consist of, or consist essentially of, the features shown in FIG. 5.

In embodiments of these cell-free expression vectors, the insulating terminator can be located between 0 and 10,000 nucleotides in a 5′ direction from the promoter, e.g., 27 to 37, or 37, nucleotides in the 5′ direction from the promoter. In some embodiments, the insulating terminator sequence is located about 10, about 15, about 20, about 25, about 30, about 35, about 40, about 45, about 50, about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10,000 nucleotides in the 5′ direction from the promoter.

In some embodiments of the cell-free expression vectors, the insulating terminator sequence is located 0 to 10,000 nucleotides, or 30 to 40 nucleotides, or 40 nucleotides, in a 3′ direction of the ori. In some embodiments, the insulating terminator sequence is located about 10, about 15, about 20, about 25, about 30, about 35, about 40, about 45, about 50, about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10,000 nucleotides in the 3′ direction of the ori.

In certain embodiments of the cell-free expression vectors, the cell-free expression vector further includes a Ribosome-binding site (RBS) and/or an Open Reading Frame (ORF).

Standard expression vectors for cell free protein synthesis (CFPS) exhibit some read-through expression of the protein of interest in common cloning strains of E. coli. This may not always be problematic for plasmid production, but can become so when the gene is toxic or confers any significant fitness defect. This expression results from transcriptional read-through from other parts of the plasmid. Accordingly, the methods provided herein reduce, avoid, or prevent read-through of product proteins when preparing plasmids for use in CFPS, but decrease, avoid, or prevent a reduction of protein synthesis of the product protein during CFPS at the commercial scale. The present disclosure solves this problem, for example, by adding insulating terminator sequences that are in the 5′ direction of the promoter and in the 3′ direction of the origin of replication (ori).

### Definitions

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid,” “oligonucleotide,” and “polynucleotide,” and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979. Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer can be a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features that allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

The term “promoter” refers to a cis-acting DNA sequence that directs RNA polymerase and other trans-acting transcription factors to initiate RNA transcription from the DNA template that includes the cis-acting DNA sequence.

The terms “target, “target sequence,” “target region,” and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem, and Mol. Biol. 26(3/4):227-259; and Owczarzy et al., 2008, Biochemistry, 47: 5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, “expression template” refers to a nucleic acid that serves as either a substrate for transcribing at least one RNA that can be translated into a polypeptide or protein or a substrate than can be translated into a polypeptide or protein. Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “translation template” refers to an RNA product of transcription from an expression template that can be used by ribosomes to synthesize polypeptide or protein.

As used herein, the term “cap” (or “5′-cap”) refers to a chemical modification of the 5′-terminus of a translation template. A cap for eukaryotic translation templates can include a guanine nucleotide connected to the mRNA via a 5′ to 5′ triphosphate linkage (“5′,5′-GpppG” or “G(5′)ppp(5′)G”). The N-7 position guanine cap can methylated (“m⁷GpppG” or “m⁷G(5)ppp(5′)G”). Translation templates that include cap can be designated by 5′,5′-GpppG-, G(5′)ppp(5′)G-, m⁷G(5′)ppp(5′)G- or m⁷GpppG-translation templates.

As used herein, “cap-dependent,” as the term modifies “translation” or “translation template,” refers to the requirement of the translation template to include a 5′-cap for efficient protein synthesis from that translation template.

As used herein, “cap-independent,” as the term modifies “translation” or “translation template,” refers to the lack of a requirement that the translation template include a 5′-cap for efficient protein synthesis from that translation template.

The term “reaction mixture,” as used herein, refers to a solution containing reagents necessary to carry out a given reaction.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215:403-10), BLAST-2, BLAST-P, BLAST-N, BLAST-X, WU-BLAST-2, ALIGN, ALIGN-2, CLUSTAL, or Megalign™ (DNASTAR™) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%).

Reference to an element by the indefinite article “a” or “an” does not exclude the possibility that more than one element is present, unless the context clearly requires that there be one and only one element. The indefinite article “a” or “an” thus usually means “at least one.”

The term “about” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” will depend upon the particular system under study.

The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows a diagram of an example showing that standard cell-free vectors can result in cell growth issues when the gene of interest that is cloned into the plasmid is toxic or causes an increased cell burden (top) and that these issues can be mitigated when an insulating terminator is used as described herein (bottom).

FIG. 2 is a diagram of an example of an unmodified pJL1 expression vector.

FIG. 3 is a diagram of an example of a modified version of the pJL1 expression vector as described herein, where the insulating terminator sequence (mpB-T1) is located immediately downstream of the origin of replication and 27 nucleotides upstream of the T7 promoter.

FIGS. 4A-4B are images of examples of Petri dishes that show that terminators in the 5′ direction of the T7 promoter reduced read through expression.

FIG. 5 is a diagram of an example of a modified version of the pJL1 expression vector, where the insulating terminator sequence (mpB-T1) is 40 nucleotides downstream of the origin of replication and 37 nucleotides upstream of the T7 promoter.

FIG. 6A are images of examples of Petri dishes that show that an alternate spacing of the insulating terminator resulted in a greater reduction of read through expression.

FIG. 6B is a bar graph that shows measured fluorescence (RFU) and culture optical density (OD) for three independent colonies chosen from each vector as described herein. Fluorescence is indicative of read through expression of the protein sfGFP from the vector. The measurement is normalized to cell culture density.

FIG. 7 is a bar graph showing that use of an insulating terminator resulted in comparable protein production as compared to experiments in which an insulating terminator was not used.

### DETAILED DESCRIPTION

Standard expression vectors for cell free protein synthesis (CFPS) may exhibit read-through expression of the protein of interest in common cloning strains of E. coli. Normally, this is not problematic for plasmid production, but can become so when the gene is toxic or confers any significant fitness defect to the cells used to generate the plasmids. This read-through expression results from transcriptional read-through from other parts of the plasmid. The methods, compositions, and kits provided herein reduce read-through of product proteins when preparing large numbers of plasmids for use in CFPS, but decrease, avoid, or prevent a reduction of protein synthesis of the product protein during CFPS at the commercial scale.

### I. Methods

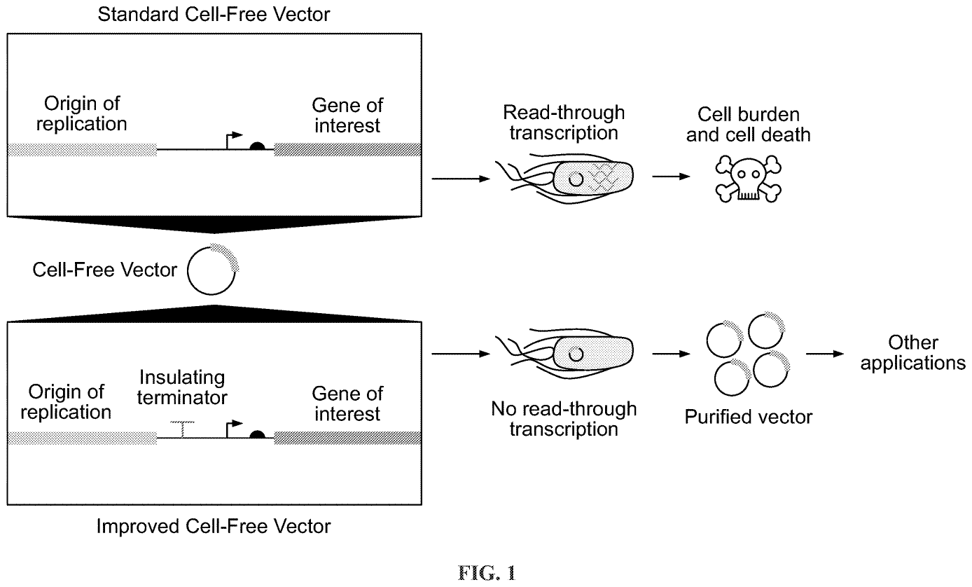

As shown in FIG. 1, standard cell-free vectors can result in cell growth issues when the gene of interest that is cloned into the plasmid is toxic or causes increased cell burden (top of FIG. 1). The present disclosure solves this problem of read through expression by adding insulating terminator sequences that are in the 5′ direction of the promoter and in the 3′ direction of the origin of replication (ori) (bottom of FIG. 1), for example.

The methods described herein can reduce read through expression by, for example, at least a 23-fold amount (that is, when compared to the parent plasmid).

### Methods for Making a Modified Cell-Free Expression Vector

In one embodiment, this disclosure provides methods of creating or modifying a cell-free expression vector to avoid, inhibit, reduce, or prevent read-through of toxic product proteins during generation of plasmids, while avoiding, decreasing, or preventing a reduction in protein synthesis of the product protein during CFPS at the commercial scale. The methods include: (a) obtaining a cell-free expression vector including (i) a promoter, (ii) an ori, (ii) a nucleic acid sequence encoding a protein or RNA, (iii) a promoter arranged to drive expression of the protein or RNA, and (iv) one or more selectable markers and (b) inserting into the cell-free expression vector an insulating terminator sequence at a location that is between 0 and 10,000 nucleotides in a 5′ direction from the promoter (that is, the last nucleotide of the terminator sequence is between 0 and 10,000 nucleotides in the 5′ direction from the first nucleotide of the promoter sequence), and is also located 0 to 10,000 nucleotides in the 3′ direction of the ori (that is, the first nucleotide sequence of the terminator sequence is 0 to 10,000 nucleotides in the 3′ direction from the last nucleotide of the ori sequence).

For example, the insulating terminator sequence can be located between 0 and 500 nucleotides in the 5′ direction from the promoter (that is, the last nucleotide of the terminator sequence is between 0 and 500 nucleotides in the 5′ direction from the first nucleotide of the promoter sequence), and can also be located 0 to 500 nucleotides in the 3′ direction of the ori (that is, the first nucleotide sequence of the terminator sequence is 0 to 500 nucleotides in the 3′ direction from the last nucleotide of the ori sequence).

### (a) Obtaining a Cell-Free Expression Vector

As described below, cell-free expression vectors are known in the art and are explained below. Known cell-free expression vectors include pJL1, pY71, p70a, pBEST, pEXP5, pT7CFE.

### (b) Inserting an Insulating Terminator Sequence

The preparation of cell-free expression vectors can be carried out using standard cloning procedures well known in the art, e.g., as described by Joseph Sambrook et al., MOLECULAR CLONING: A LABORATORY MANUAL (Cold Springs Harbor 1989), including the Nov. 18, 2014 updated version of Sambrook, and U.S. Pat. No. 4,237,224 to Cohen and Boyer, which are hereby incorporated by reference in their entireties. For example, standard cloning procedures, well known to one skilled in the art, may be used to insert a terminator sequence according to the desired spacing as noted above (i.e., an insulating terminator sequence that is located between 0 and 10,000 nucleotides in the 5′ direction from the promoter (that is, the last nucleotide of the terminator sequence is between 0 and 10,000 nucleotides in the 5′ direction from the first nucleotide of the promoter sequence), and is also located 0 to 10,000 nucleotides in the 3′ direction of the ori (that is, the first nucleotide sequence of the terminator sequence is 0 to 10,000 nucleotides in the 3′ direction from the last nucleotide of the ori sequence).

For example, the insulating terminator sequence can be located between 0 and 500 nucleotides in the 5′ direction from the promoter (that is, the last nucleotide of the terminator sequence is between 0 and 500 nucleotides in the 5′ direction from the first nucleotide of the promoter sequence), and can also be located 0 to 500 nucleotides in the 3′ direction of the ori (that is, the first nucleotide sequence of the terminator sequence is 0 to 500 nucleotides in the 3′ direction from the last nucleotide of the ori sequence). In some embodiments, the insulating terminator sequence can be located about 50 nucleotides in the 5′ direction from the promoter (e.g., about 20, about 25, about 30, about 35, about 40, about 45, about 50 nucleotides in the 5′ direction from the promoter). In some instances, the insulating terminator sequence can be located about 27 to 37 or 35 to 37, or 37 nucleotides in the 5′ direction from the promoter. In certain embodiments, the insulating terminator sequence is located 0 to 50 nucleotides in a 3′ direction of the ori, (e.g., about 5, about 10, about 15, about 20, about 25, about 30, about 35, about 40, about 45, about 50 nucleotides in the 3′ direction of the ori). In some instances, the insulating terminator sequence is located 30 to 40 nucleotides, or 40 nucleotides, in the 3′ direction of the ori.

### Methods of Performing Protein Synthesis In Vitro

The disclosure also provides methods of performing protein synthesis in vitro the method comprising synthesizing protein or RNA in vitro using a cell-free expression vector, wherein the cell-free expression vector includes: (i) an origin of replication (ori); (ii) a nucleic acid encoding protein or RNA to be synthesized; (iii) a promoter arranged to drive expression of the protein or RNA; (iv) an insulating terminator sequence located between 0 and 10,000 nucleotides in a 5′ direction from the promoter (that is, the last nucleotide of the terminator sequence is between 0 and 10,000 nucleotides in the 5′ direction from the first nucleotide of the promoter sequence), and is also located 0 to 10,000 nucleotides in the 3′ direction of the ori (that is, the first nucleotide sequence of the terminator sequence is 0 to 10,000 nucleotides in the 3′ direction from the last nucleotide of the ori sequence); and (iv) one or more selectable markers.

For example, the insulating terminator sequence can be located between 0 and 500 nucleotides in the 5′ direction from the promoter (that is, the last nucleotide of the terminator sequence is between 0 and 500 nucleotides in the 5′ direction from the first nucleotide of the promoter sequence), and can also be located 0 to 500 nucleotides in the 3′ direction of the ori (that is, the first nucleotide sequence of the terminator sequence is 0 to 500 nucleotides in the 3′ direction from the last nucleotide of the ori sequence).

(a) Providing a Nucleic Acid that Encodes a Protein or RNA Product

A nucleic acid molecule is any nucleic acid sequence that encodes a desired protein or RNA product.

(b) Preparing a Cell-Free Expression Vector from the Nucleic Acid

As provided herein, a desired source nucleic acid is cloned into the modified cell-free expression vector as described above. Generally, the use of cell-free expression vectors to produce and isolate a protein of interest involves inserting a source nucleic acid molecule (for protein or RNA) into a cell-free expression vector to which the molecule is heterologous (i.e., not normally present). One or more desired nucleic acid molecules encoding one or more proteins may be inserted into the vector. When multiple nucleic acid molecules are inserted, the multiple nucleic acid molecules may encode the same or different enzymes. The heterologous nucleic acid molecule is inserted into the expression system or vector in proper sense (5′→3′) orientation relative to the promoter and any other 5′ regulatory molecules, and correct reading frame. The preparation of the cell-free expression vectors can be carried out using standard cloning procedures well known in the art, e.g., as described by Joseph Sambrook et al., MOLECULAR CLONING: A LABORATORY MANUAL (Cold Springs Harbor 1989), including the Nov. 18, 2014 updated version of Sambrook, and, e.g., in U.S. Pat. No. 4,237,224 to Cohen and Boyer, which are hereby incorporated by reference in their entireties.

### (c) Synthesizing the Product Protein In Vitro Using the Cell-Free Expression Vector

The modified cell-free expression vectors including the nucleic acid molecule is then introduced by means of transformation and replicated in a suitable host cell. The components, systems, and methods disclosed herein may be applied to, or adapted to cell-free protein synthesis methods as known in the art. See, for example, U.S. Pat. Nos. 5,478,730; 5,556,769; 5,665,563; 6,168,931; 6,548,276; 6,869,774; 6,994,986; 7,118,883; 7,186,525; 7,189,528; 7,235,382; 7,338,789; 7,387,884; 7,399,610; 7,776,535; 7,817,794; 8,703,471; 8,298,759; 8,715,958; 8,734,856; 8,999,668; and 9,005,920. See also U.S. Published Application Nos. 2018/0016614, 2018/0016612, 2016/0060301, 2015-0259757, 2014/0349353, 2014-0295492, 2014-0255987, 2014-0045267, 2012-0171720, 2008-0138857, 2007-0154983, 2005-0054044, and 2004-0209321. See also U.S Published Application Nos. 2005-0170452; 2006-0211085; 2006-0234345; 2006-0252672; 2006-0257399; 2006-0286637; 2007-0026485; 2007-0178551. See also Published PCT International Application Nos. 2003/056914; 2004/013151; 2004/035605; 2006/102652; 2006/119987; and 2007/120932. See also Jewett, M. C., Hong, S. H., Kwon, Y. C., Martin, R. W., and Des Soye, B. J. 2014, “Methods for improved in vitro protein synthesis with proteins containing non-standard amino acids,” U.S. Patent Application Ser. No. 62/044,221; Jewett, M. C., Hodgman, C. E., and Gan, R. 2013, “Methods for yeast cell-free protein synthesis,” U.S. Patent Application Ser. No. 61/792,290; Jewett, M. C., J. A. Schoborg, and C. E. Hodgman. 2014, “Substrate Replenishment and Byproduct Removal Improve Yeast Cell-Free Protein Synthesis,” U.S. Patent Application Ser. No. 61/953,275; and Jewett, M. C., Anderson, M. J., Stark, J. C., Hodgman, C. E. 2015, “Methods for activating natural energy metabolism for improved yeast cell-free protein synthesis,” U.S. Patent Application Ser. No. 62/098,578. See also Guarino, C., & DeLisa, M. P. (2012). A prokaryote-based cell-free translation system that efficiently synthesizes glycoproteins. Glycobiology, 22(5), 596-601. The contents of all of these references are incorporated in the present application by reference in their entireties.

### II. Cell-Free Expression Vectors

The disclosure further provides modified cell-free expression plasmids, methods for making them, and methods for using them. As used herein, the term “vector” refers to a nucleic acid molecule capable of transporting another nucleic acid to which it has been linked. One type of vector is a “plasmid,” which refers to a circular double-stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” are used interchangeably.

While cell-free expression vectors are well known in the art, we briefly describe below the required and optional features of the cell-free expression vectors.

### Necessary Features of Cell-Free Expression Vectors

### Insulating Terminator Sequences

Provided herein are expression vectors that include an insulating terminator sequence. The term “insulating” means that the terminator sequence is located between 0 and 10,000 nucleotides in the 5′ direction from the promoter (that is, the last nucleotide of the terminator sequence is between 0 and 10,000 nucleotides in the 5′ direction from the first nucleotide of the promoter sequence), and is also located 0 to 10,000 nucleotides in the 3′ direction of the ori (that is, the first nucleotide sequence of the terminator sequence is 0 to 10,000 nucleotides in the 3′ direction from the last nucleotide of the ori sequence).

For example, the insulating terminator sequence can be located between 0 and 500 nucleotides in the 5′ direction from the promoter (that is, the last nucleotide of the terminator sequence is between 0 and 500 nucleotides in the 5′ direction from the first nucleotide of the promoter sequence), and can also be located 0 to 500 nucleotides in the 3′ direction of the ori (that is, the first nucleotide sequence of the terminator sequence is 0 to 500 nucleotides in the 3′ direction from the last nucleotide of the ori sequence).

Examples of terminator sequences are described in Chen, Y J., Liu, P., Nielsen, A. et al. Characterization of 582 natural and synthetic terminators and quantification of their design constraints. Nat Methods 10, 659-664 (2013); Guillaume Cambray, Joao C. Guimaraes, Vivek K. Mutalik, Colin Lam, Quynh-Anh Mai, Tim Thimmaiah, James M. Carothers, Adam P. Arkin, Drew Endy, Measurement and modeling of intrinsic transcription terminators, Nucleic Acids Research, Volume 41, Issue 9, 1 May 2013, Pages 5139-5148; Diana G Calvopina-Chavez, Mikaela A Gardner, Joel S Griffitts, Engineering efficient termination of bacteriophage T7 RNA polymerase transcription, G3 Genes|Genomes|Genetics, Volume 12, Issue 6, June 2022, jkac070, each of which are hereby incorporated in their entireties by reference.

Numerous examples of specific terminator sequences (SEQ ID NOs: 1 to 581 and SEQ ID NO:820) are listed in Appendix 1, which is appended to this application (parts.igem.org/Terminators/Catalog). This list contains a useful, but non-exhaustive, list of terminators that may be used in certain embodiments. The iGEM parts registry name, descriptions (such as natural contexts or synthetic origins), and sequences are provided.

In some embodiments, the terminator sequence is mpB-T1, rrnB-T1, L3S2P21, or L3S2P56 (or a terminator sequence having at least 70%, 75%, 80%, 85%, 90%, 95%, 96%, 97%, 98%, 99%, or 100% sequence similarity to one of these terminator sequences). In some embodiments, more than one terminator sequence is included in the cell-free expression vector. In some embodiments, only one terminator sequence is included in the cell-free expression vector.

### Promoters

For the purposes of expressing a nucleic acid sequence encoding one or more desired proteins, different promoters can be used to produce genes at various levels and rates. Depending upon the host system utilized, any one of a number of suitable promoters may be used. Promoters are well known in the art to one of skill in the art. For instance, T7 phage promoter, lac promoter, trp promoter, recA promoter, ribosomal RNA promoter, Sp6 promoter, araBad promoter, pTac promoter, or J23119 promoter.

Numerous examples of additional promoter sequences (SEQ ID NOs: 582 to 814 and SEQ ID NO:819) are also listed in Appendix 2, which is appended to this application. This list contains a useful, but non-exhaustive, list of promoters that may be used in certain embodiments. The iGEM parts registry name, descriptions (such as natural contexts or synthetic origins), and sequences are provided.

Origin of Replication (ori)

The ori is the place where DNA replication begins, enabling a plasmid to reproduce itself as it must to survive within cells. The replicons of plasmids are generally different from those used to replicate the host's chromosomal DNA, but they still rely on the host machinery to make additional copies. In some embodiments, more than one ori is included in the cell-free expression vector. In some embodiments, only one ori is included in the cell-free expression vector.

### Selectable Marker(s)

Selectable marker(s) are well known in the art and can be categorized into those based on resistance genes that confer the ability to grow in the presence of toxic compounds such as antibiotics or herbicides that kill or otherwise compromise untransformed tissue (negative selection). Alternatively, a range of positive selection systems are available which provide transformed tissues with an enhanced ability to utilize, for example, an unusual carbohydrate or amino acid supply and thus enrich the culture for transformed tissue expressing the marker gene.

In some embodiments, the selectable marker is Kanamycin, Spectinomycin, Streptomycin, Ampicillin, Carbenicillin, Bleomycin, Erythromycin, Polymyxin B, Tetracycline, or Chloramphenicol.

### Open Reading Frame (ORF)

As would be recognized by one skilled in the art, the cell-free expression vectors include an open reading frame.

### Ribosomal Binding Site (RBS)

There are other specific initiation signals required for efficient gene transcription and translation in prokaryotic cells that can be included in the nucleic acid construct to maximize peptide production, e.g., the Shine-Dalgarno ribosome binding site. Depending on the vector system and host utilized, any number of suitable transcription and/or translation elements, including constitutive, inducible, and repressible promoters, as well as minimal 5′ promoter elements, enhancers or leader sequences may be used. For a review on maximizing gene expression see Roberts and Lauer, “Maximizing Gene Expression on a Plasmid Using Recombination In Vitro” Methods in Enzymology 68:473-82 (1979), which is hereby incorporated by reference in its entirety.

### Optional Features of the Plasmid

In some embodiments, the cell-free expression vector includes a multiple cloning site. Alternatively, the cell-free expression vector does not include a multiple cloning cite. When included, a multiple cloning site, as recognized by one of skill in the art, is a short segment of DNA that contains many (up to ~20) restriction sites. For example, any of the following restriction sites: SgrAI, SrfI, XmaI, SpeI, BamHI, BglII, Xholl, SacII, RsrII, PacI, NruI, NotI, NdeI, MscI, MluI, KpnI, Fse1, BssHII, BsrGI, BspEI, BclI, BbvC1, Pmel, BssHII, AscI, XbaI.

In some embodiments, the cell-free expression vector includes one or more transcriptional or translational regulation sites. For example, transcription factor operator sites.

In some embodiments, the cell-free expression vector includes one or more inducer elements that can be utilized for the induction of gene expression. For example, LacO (for use with Lactose or IPTG), P(BAD) (for use with arabinose), or Tet (for use with tetracycline).

### III. Kits

Also provided herein in some embodiments are kits for use in a method of creating or modifying a cell-free expression vector or kits for performing protein or RNA synthesis in vitro.

In some embodiments, the kits for creating or modifying a cell-free expression vector include: (a) a cell-free expression vector comprising (i) an origin of replication (ori), (ii) a nucleic acid sequence encoding a protein or RNA, (iii) a promoter arranged to drive expression of the protein or RNA, and (iv) one or more selectable markers; and (b) an insulating terminator sequence that is to be inserted at a location between 0 and 10,000 nucleotides in a 5′ direction from the promoter in the cell-free expression vector; and (c) cloning reagents. In certain instances, the kits for performing protein or RNA synthesis in vitro, the kit comprising: (a) reagents for cell-free protein or RNA synthesis; and (b) a cell-free expression vector comprising (i) an origin of replication (ori); (ii) a nucleic acid encoding protein or RNA to be synthesized; (iii) a promoter arranged to drive expression of the protein or RNA; (iv) an insulating terminator sequence that is located between 0 and 10,000 nucleotides in a 5′ direction from the promoter; and (iv) one or more selectable markers.

For example, the insulating terminator sequence can be located between 0 and 500 nucleotides in the 5′ direction from the promoter (that is, the last nucleotide of the terminator sequence is between 0 and 500 nucleotides in the 5′ direction from the first nucleotide of the promoter sequence), and can also be located 0 to 500 nucleotides in the 3′ direction of the ori (that is, the first nucleotide sequence of the terminator sequence is 0 to 500 nucleotides in the 3′ direction from the last nucleotide of the ori sequence).

The kit can include an unmodified cell-free expression vector (e.g., pJL1, pY71, p70a, pBEST, pEXP5, pT7CFE), a terminator sequence (e.g., rnpB-T1, rrnB-T1, L3S2P21, L3S2P56), and the necessary cloning reagents to insert the terminator sequence. The preparation of the cell-free expression vectors can be carried out using standard cloning reagents (e.g., dNTPs, DNA polymerase, buffers, DNA ligase, DNA restriction endonuclease, DNA exonuclease) and procedures that are well known in the art, e.g., as described by Joseph Sambrook et al., MOLECULAR CLONING: A LABORATORY MANUAL (Cold Springs Harbor 1989), including the Nov. 18, 2014 updated version of Sambrook, and U.S. Pat. No. 4,237,224 to Cohen and Boyer, which are hereby incorporated by reference in their entireties.

In some embodiments, provided herein is a kit that includes the modified plasmids as described above and the reagents for cell-free expression, e.g., a “CFPS reaction mixture.” In some embodiments, a “CFPS reaction mixture” typically contains a crude or partially-purified cell extract (e.g., a yeast or bacterial extract), an RNA translation template, and a suitable reaction buffer for promoting cell-free protein synthesis from the RNA translation template.

As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are, for example at least 60% free, at least 75% free, at least 90% free, or at least 95% free from other components with which they are naturally associated.

In some embodiments, the cells used to derive the crude or partially purified extract are selected based on the presence or absence of specific endogenous biochemical pathways and/or engineered biochemical pathways. For example, cells that direct carbon flux, prevent or minimize side product formation, and prevent or minimize promiscuous background activity can be advantageous as compared to other cells. In some embodiments, the cell is a prokaryotic cell (e.g., bacterial cell) or a eukaryotic cell (e.g., a yeast cell). In some embodiments, the cell is a prokaryotic cell and comprises and E. coli cell. In some embodiments, the E. coli cell comprises a modified E. coli cell, such as BL21, JST07, MB263, MP263sucD, and JC01. In some embodiments, the E. coli cell comprises JST07.

As used herein, “translation template” for a polypeptide refers to an RNA product of transcription from an expression template that can be used by ribosomes to synthesize polypeptides or proteins. For example, a CFPS reaction mixture may include an expression template, a translation template, or both an expression template and a translation template. The expression template serves as a substrate for transcribing at least one RNA that can be translated into a sequence-defined biopolymer (e.g., a polypeptide or protein). The translation template is an RNA product that can be used by ribosomes to synthesize the sequence-defined biopolymer. In certain embodiments, the platform comprises both the expression template and the translation template.

The CFPS reaction mixture may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

The reaction mixture may include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others. The reaction mixture may include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The reaction mixture may include any organic cation suitable for CFPS. In certain aspects, the organic cation can be a polyamine, such as spermidine or putrescine, among others. In some embodiments, polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The reaction mixture may include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In some implementations, the concentration of inorganic cations can be within the specific range from about 4 mM to about 9 mM. In some implementations, the concentration of inorganic cations can be within the range from about 5 mM to about 7 mM.

The reaction mixture may include endogenous NTPs (i.e., NTPs that are present in the cell extract) and or exogenous NTPs (i.e., NTPs that are added to the reaction mixture). In certain aspects, the reaction use ATP, GTP, CTP, and UTP. In certain aspects, the concentration of individual NTPs is within the range from about 0.1 mM to about 2 mM.

The reaction mixture may include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

As recognized by one skill in the art, components for a reaction mixture may be stored separately in separate containers, each containing one or more of the total components. Components may be packaged separately for commercialization and useful commercial kits may contain one or more of the reaction components for a reaction mixture.

### EXAMPLES

The invention is further described in the following examples, which do not limit the scope of the invention described in the claims.

### Example 1: Insulating Terminators Reduced Read-Through Expression

We first assessed if inclusion of a terminator sequence could reduce read through expression. To do this, we split the pJL1 vector into two pieces of synthetic DNA (backbone and insert). We then designed different inserts with different combinations of the T7 promoter and different terminators that were in the 5′ direction of the T7 promoter (see, Table 1 below showing the various terminators tested (note Sample #6 was discarded)).

TABLE 1

| Sample # | Promoter | Terminator | Condition |
|---|---|---|---|
| 1 | T7 | None | Positive Control |
| 2 | None | None | Negative Control |
| 3 | T7 | rnpB | Endogenous Terminator |
| 4 | T7 | rrnB | Endogenous Terminator |
| 5 | T7 | T7 | Phage Terminator |
| 7 | T7 | M13 & rrnD | Phage Terminator |
| 8 | T7 | L3S2P21 | Synthetic Endogenous Terminator |
| 9 | T7 | L3S2P56 | Synthetic Endogenous Terminator |

The terminator design is shown below. A portion of the sequence of the pJL1 vector is shown below (bold text is the end of the pMB1 origin of replication and italicized text is the T7 RNA polymerase promoter):

(SEQ ID NO: 815)
ctgtcgggtttcgccacctctgacttgagcgtcgatttttgtgatgc
tcgtcaggggggcggagcctatggaaaaacgccagcaacgcgatccc
gcgaaattaatacgactcactatag.

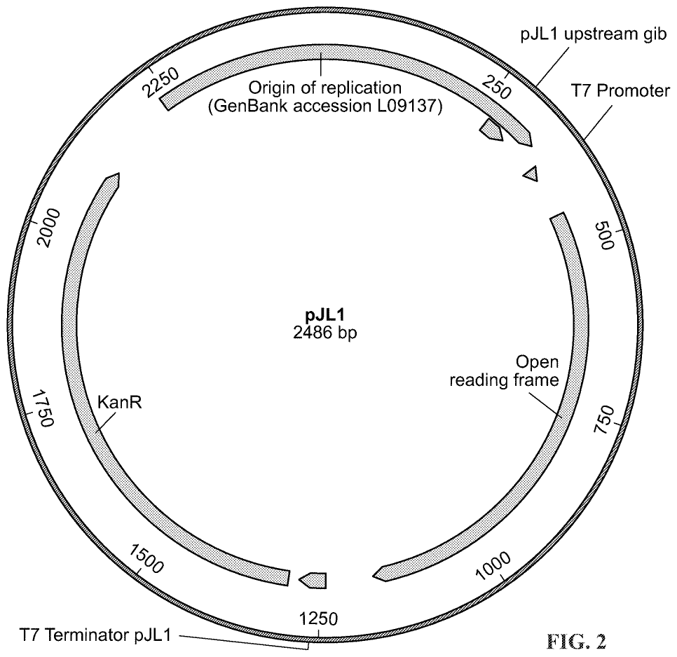

FIG. 2 shows a full map of the unmodified pJL1 expression vector.

In these initial experiments, the terminators were placed immediately downstream of the origin of replication and 27 nucleotides upstream of the T7 promoter, as shown below (bold text is the end of the pMB1 origin of replication and italicized text is the T7 RNA polymerase promoter; and the bold and italicized text is the terminator sequence):

(SEQ ID NO: 816)
ctgtcgggtttcgccacctctgacttgagcgtcgatttttgtgatgc
tcgtcaggggggcggagcctatggaaaccggcttatcggtcagtttc
acctgatttacgtaaaaacccgcttcggcgggtttttgcttttggag
gggcagaaagatgaatgactgtccacgacgctatacccaaaagaaaa
acgccagcaacgcgatcccgcgaaattaatacgactcactatag.

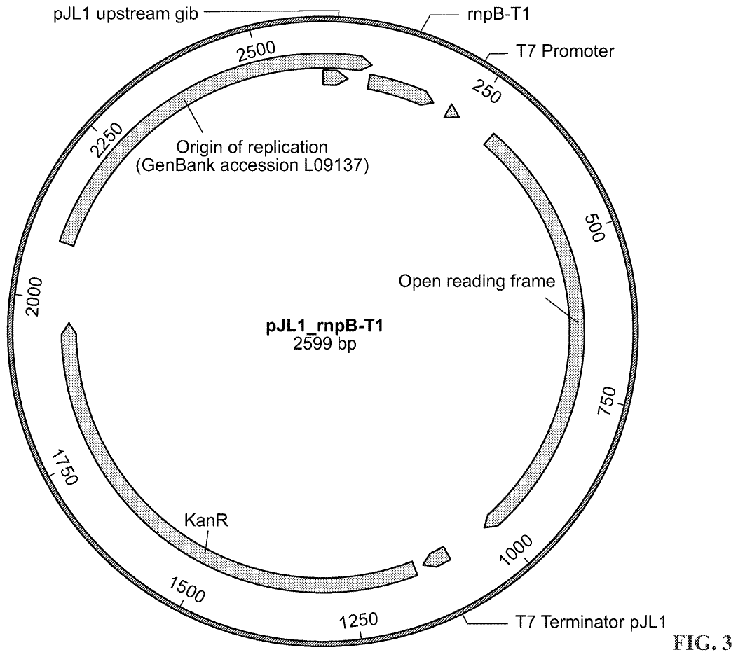

FIG. 3 shows a full map of a modified pJL1 expression vector, where the insulating terminator rnpB-T1 sequence was inserted into the pJL1 vector immediately 3′ of the origin of replication and 27 nucleotides 5′ of the T7 promoter.

In general, the T7 promoter regulates the expression of GFP, so if read-through is observed it can be visualized by fluorescent readout. Gibson assembly reactions were performed and NEB5a E. coli cells were transformed with the vectors and grown overnight at 37° C. The next day, the plates were imaged using a fluorescent imager (to visualize GFP expression) and with a cell phone (to visualize cell grown and roughly gauge transformation efficiency).

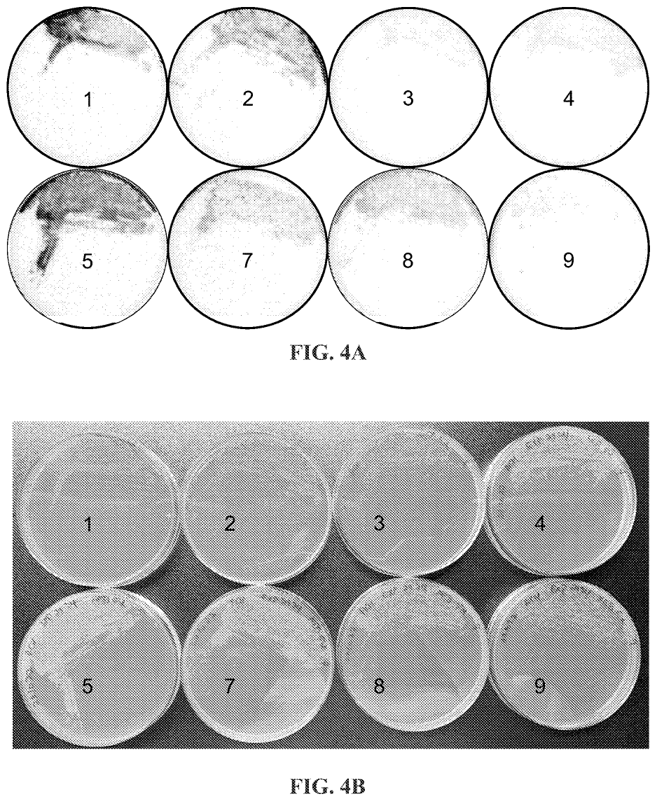

FIG. 4A shows the GFP expression in the cells cultured in Petri dishes. Condition 2 (which lacks a T7 promoter) showed appreciable GFP fluorescence, indicating that read through expression is due to run-on transcription from somewhere upstream of the gene of interest (GOI). It also indicated that read through expression was not due to endogenous RNA polymerases recognizing the T7 promoter. In Samples 3, 4, 8, and 9, endogenous terminators appeared to be better at inhibiting read through expression than phage terminators (Samples 5 and 7), because Sample 5 showed considerable GFP expression, and Sample 7 had less, but still significant GFP expression.

FIG. 4B shows an overall cell growth for the plates shown in FIG. 4A. The large number of colonies in each condition indicated that the lack of fluorescence in Samples #3, 4, 8, and 9 are not due to a lack of cell growth, but rather the specific decrease in fluorescence due to the presence of an insulating terminator.

### Example 2: Modification of Terminator Location to Improve Plasmid Production Yields

Initial placement of terminators in Example 1 reduced plasmid yields. NEB5a cells harboring the plasmids of interest were cultured and then a large scale plasmid purification was performed. Yields were determined using absorbance (A280). For the plasmids with terminators inserted, the purified plasmid yields were consistently only about 25% of the yields of the unmodified plasmid. As a result, we altered the spacing of the terminator in the insert design to see if this alteration could improve the plasmid yields (see, Table 2 below). Specifically, in this set of experiments, the terminators were placed 40 nucleotides 3′ to the origin of replication and 37 nucleotides 5′ to the T7 promoter, as shown below (bold text is the end of the pMB1 origin of replication and italicized text is the T7 RNA polymerase promoter; and the bold and italicized text is the terminator sequence):

(SEQ ID NO: 817)
ctgtcgggtttcgccacctctgacttgagcgtcgatttttgtgatgc
tcgtcaggggggcggagcctatggaaaaacgccagcaacgcggcctt
tttacggttcctggccttttccggcttatcggtcagtttcacctgat
ttacgtaaaaacccgcttcggcgggtttttgcttttggaggggcaga
aagatgaatgactgtccacgacgctatacccaaaagaaagctggcct
tttgctcacatgttcttatcccgcgaaattaatacgactcactatag.

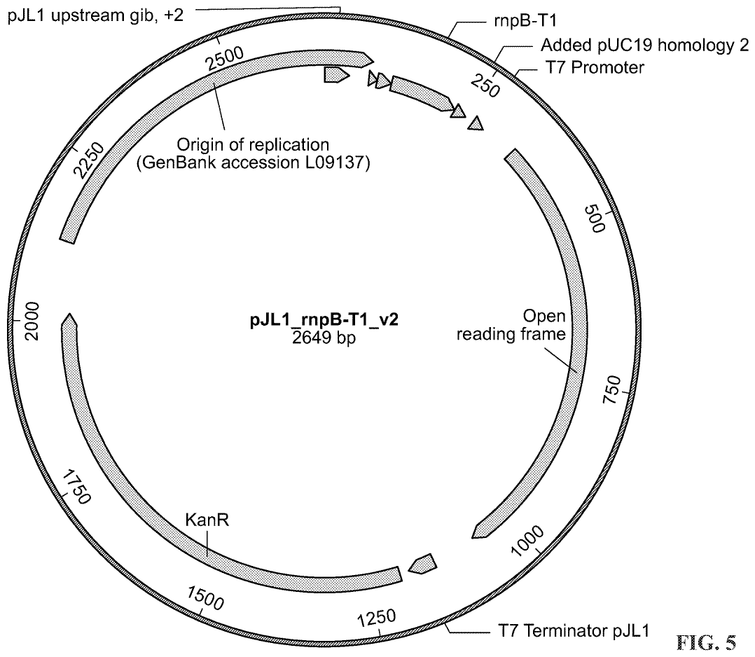

FIG. 5 shows a full map of the modified pJL1 expression vector, where the insulating terminator is 40 nucleotides 3′ of the origin of replication and 37 nucleotides 5′ of the T7 promoter.

TABLE 2

| Sample # | Promoter | Terminator | Condition |
|---|---|---|---|
| 10 | T7 | None | Positive Control |
| 11 | None | None | Negative Control |
| 12 | T7 | rnpB | Altered terminator location |
| 13 | T7 | rrnB | Altered terminator location |
| 14 | T7 | L3S2P21 | Altered terminator location |
| 15 | T7 | L3S2P56 | Altered terminator location |

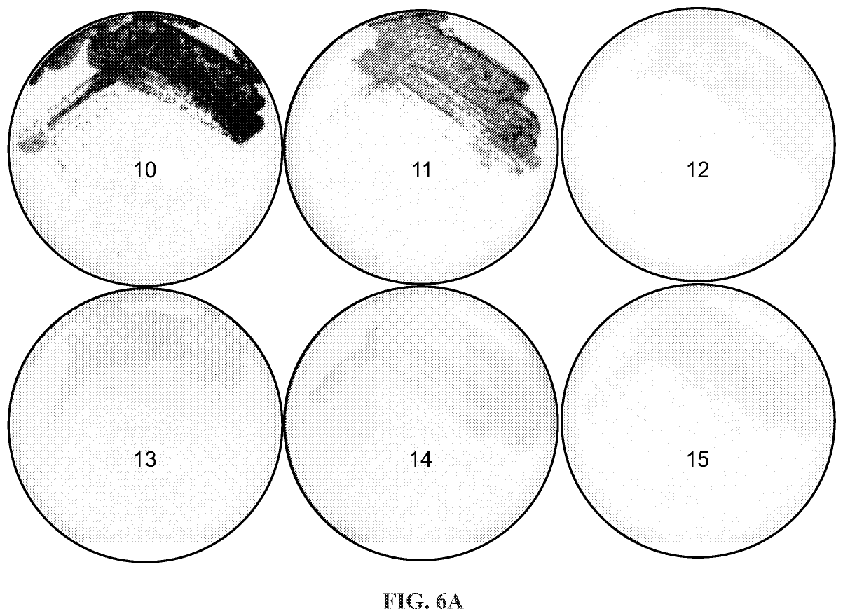

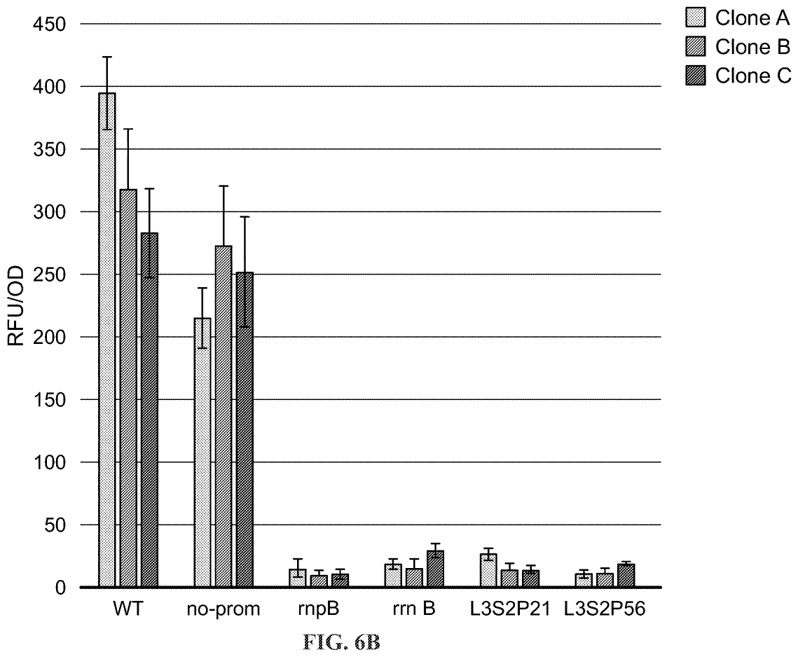

As in Example 1, read through expression was greatly reduced with the endogenous terminators (see, e.g., Samples 12-15 in FIG. 6A). Similar results to those seen in the solid culture were observed in liquid culture as well. That is, we observed that endogenous terminators nearly eliminated read through expression of GFP in liquid culture tests using standard techniques. As shown in the bar graph of FIG. 6B, the wildtype (WT) and no promoter showed significant GFP expression as evidenced by fluorescence (high RFU/OD), whereas the insertion of the terminators mpB, rrnB, L3S2P21, and L3S2P56 all significantly reduced GFP expression. The results were consistent across three clones, A, B, and C.

We also cultured NEB5a cells harboring the plasmids of interest and performed large-scale plasmid purification. Yields were again determined using absorbance (A280). As compared to Example 1, for the vectors with terminators inserted in the new location, the purified plasmid yields were comparable to the unmodified plasmid. Overall, this example shows that terminators need to be placed in the correct location to enable comparable plasmid yields, but still achieve the goal of decreasing leaky or read-through expression of the downstream open reading frame.

### Example 3: Modified Plasmids Exhibit GFP Production in CFPS Comparable to GFP Production in the Original Plasmid

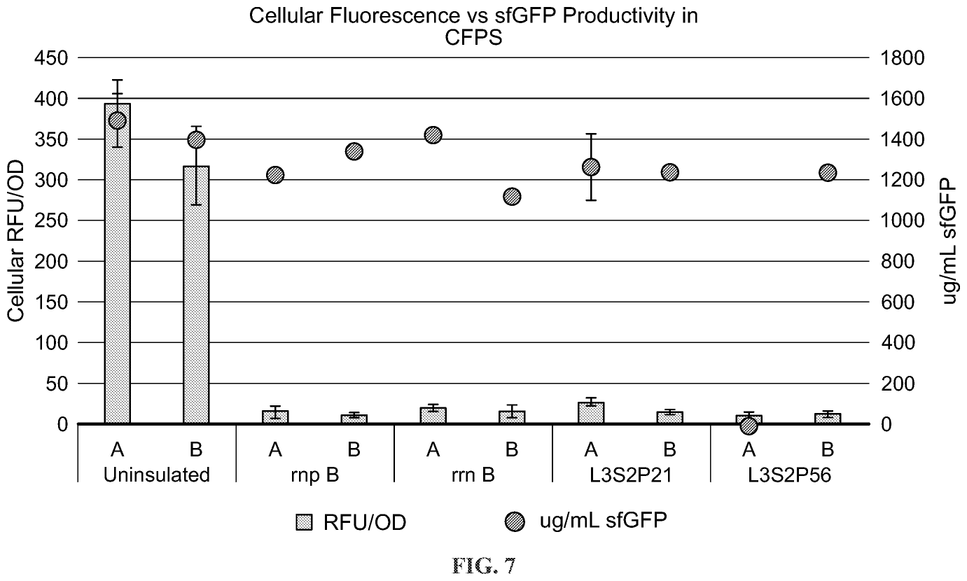

Purified plasmids were evaluated in cell-free protein synthesis (CFPS) for their ability to produce GFP. Two independent plasmid isolates (A and B) were tested for each design. In FIG. 7, the bars indicate a measurement of GFP expression in the strain used for plasmid production. We observed a similar optical density (OD) for all the different vectors, but variable expression of GFP. When a terminator was not present, a significant fluorescence was observed due to the expression of GFP. When the terminator was absent, little GFP expression was observed. Data was normalized to the concentration of cells in each well, so that samples are cross-comparable.

The circles in FIG. 7 represent the GFP production from the plasmid in a cell free protein synthesis system. All conditions yield GFP expression. As shown in FIG. 7, the modified plasmids from Example 2 showed comparable protein production (ug/mL sfGFP) to the original vector (uninsulated). Overall, this example shows that plasmids modified with insulating terminators provide similar yields when used in cell-free protein synthesis, making them fully compatible with this expression systems while making it easier to prepare these plasmids by decreasing background expression of downstream open reading frames.

### APPENDICES

APPENDIX 1
Terminator Sequences

| | | | SEQ |
|---|---|---|---|
| | | | ID |
| Name | Description | Sequence | NO: |
| ECK120029600 | spy | TTCAGCCAAAAAACTTAAGACCGCCGG | 1 |
| | | TCTTGTCCACTACCTTGCAGTAATGCGG | |
| | | TGGACAGGATCGGCGGTTTTCTTTTCTC | |
| | | TTCTCAA | |
| ECK120033737 | thrLABC | ggaaacacagAAAAAAGCCCGCACCTGACA | 2 |
| | | GTGCGGGCTTTTTTTTTcgaccaaagg | |
| pheA-1 | pheA | gacgaacaaTAAGGCCTCCCAAATCGGGGG | 3 |
| | | GCCTTTTTTATTgaTaacaaaa | |
| ECK120034435 | secG-leuU | CTCGGTACCAAATTCCAGAAAAGAGAC | 4 |
| | | GCTGAAAAGCGTCTTTTTTCGTTTTGGT | |
| | | CC | |
| ECK120033736 | hisLGDCBH | aacgcatgagAAAGCCCCCGGAAGATCACCT | 5 |
| | AFI | TCCGGGGGCTTTtttattgcgc | |
| ECK120010818 | garPLRK- | GTCAGTTTCACCTGTTTTACGTAAAAAC | 6 |
| | rnpB | CCGCTTCGGCGGGTTTTTACTTTTGG | |
| ECK125109870 | metZWV | ccaattattgAACACCCTAACGGGTGTTTTTT | 7 |
| | | TGTTTctggtctccc | |
| ECK120015440 | lpdpdhR- | tccggcaattAAAAAAGCGGCTAACCACGCC | 8 |
| | aceEF-lpd | GCTTTTTTtacgtctgca | |
| BBa_B0062-R | rrnC | cagataaaaaaaatccttagctttcgctaaggatgatttct | 9 |
| ECK120010799 | csrC | gttatgagtcAGGAAAAAAGGCGACAGAGTA | 10 |
| | | ATCTGTCGCCTTTTTTCTTtgcttgcttt | |
| ECK120010876 | creABCDcreD | taaggttgaaAAATAAAAACGGCGCTAAAAA | 11 |
| | | GCGCCGTTTTTTTTgacggtggta | |
| ECK120015170 | rplM-rpsI | ACAATTTTCGAAAAAACCCGCTTCGGC | 12 |
| | | GGGTTTTTTTATAGCTAAAA | |
| ECK120010869 | rplJL- | taacgtaaaaACCCGCTTCGGCGGGtttttttatg | 13 |
| | rpoBCrplKAJL- | | |
| | rpoBCrpoBC | | |
| BBa_B0010 | rrnB T1 | ccaggcatcaaataaaacgaaaggctcagtcgaaagactgggc | 14 |
| | | ctttcgttttatctgttgtttgtcggtgaacgctctc | |
| ECK120017009 | ihfApheMST- | GATCTAACTAAAAAGGCCGCTCTGCGG | 15 |
| | ihfA | CCTTTTTTCTTTTCACT | |
| ECK120051401 | xapR | CGCAGATAGCAAAAAAGCGCCTTTAGG | 16 |
| | | GCGCTTTTTTACATTGGTGG | |
| ECK120010855 | osmE | GTAACAACGGAAACCGGCCATTGCGCC | 17 |
| | | GGTTTTTTTTGGCCT | |
| ECK120010850 | clpPX | agttaaccaaAAAGGGGGGATTTTATCTCCC | 18 |
| | | CTTTaatttttcct | |
| thr | thr | AAAAAAGCCCGCACCTGACAGTGCGGG | 19 |
| | | CTTTTTTTTT | |
| ECK120035137 | istR-listR-2 | AGGCGACTGACGAAACCTCGCTCCGGC | 20 |
| | | GGGGTTTTTTGTTATCTGCA | |
| ECK120051382 | rrlB-rrfBrrsB- | caggcatcaaATAAAACGAAAGGCTCAGTC | 21 |
| | gltT-rrlB-rrfB | GAAAGACTGGGCCTTTCGTTTTATctgttgtt | |
| | | tg | |
| ECK120035133 | rphrph-pyrE | ACTGATTTTTAAGGCGACTGATGAGTC | 22 |
| | | GCCTTTTTTTTGTCT | |
| tonB_P14 | tonB/P14 | CCTGTTGAGTAATAGTCAAAAGCCTCC | 23 |
| | | GGTCGGAGGCTTTTGACTTTCTGCTTAC | |
| ECK120023928 | proS | GCTGATGCCAGAAAGGGTCCTGAATTT | 24 |
| | | CAGGGCCCTTTTTTTACATGGATTG | |
| BBa_J61053 | fmn T1 | gcttgctgaggatcctaaagccccgaattttttataaattcggggc | 25 |
| | | tttttt | |
| ECK120016882 | rpsT | TGTGAAAAAGCCCGCGCAAGCGGGTTT | 26 |
| | | TTTTATG | |
| pyrBI | pyrBI | AGCCCCTCAATCGAGGGGCTTTTTTTTG | 27 |
| | | C | |
| ECK120026481 | arcA | TACCACCGTCAAAAAAAACGGCGCTTT | 28 |
| | | TTAGCGCCGTTTTTATTTTTCAACCTT | |
| ECK120015444 | seqA-pgm | acatttaataAAAAAAGGGCGGTCGCAAGAT | 29 |
| | | CGCCCTTTTTTacgtatgaca | |
| ECK120010782 | fhuE | ACCTGTAAAAAAGGCAGCCATCTGGCT | 30 |
| | | GCCTTAGTCTCCCCA | |
| ilvBN | ilvBN | AAGACCCCCGCACCGAAAGGTCCGGGG | 31 |
| | ilvGEDA | GTTTTTTTT | |
| ECK120016586 | infA | AAGAACGAGTAAAAGGTCGGTTTAACC | 32 |
| | | GGCCTTTTTATTTTGTGA | |
| ECK120010797 | crrptsHI-crr | cagtgaaaaaTGGCGCCCATCGGCGCCAttttttt | 33 |
| | | atg | |
| ECK120014970 | valS | TGAAAACGAAGGCCGGAGCATGCTCCG | 34 |
| | | GCCTTTTTTATCTCTTACA | |
| ECK120015452 | nagE | caacaatgacAAGCGGTGGAGATCTTCTCTG | 35 |
| | | CCGCTTtttttttcat | |
| ECK120010863 | atpBEFHAG | AAGCACAAAAGCCAGTCTGGAAACAGG | 36 |
| | DCatpIBEFH | CTGGCTTTTTTTTGCG | |
| | AGDC | | |
| ECK120010815 | gadBC | GTTATAAGACAAGGGAGCGATAATTCA | 37 |
| | | TCGCTCCCTTTTTCGTGCTT | |
| ECK120010867 | xapAB | AAAAGGATTCGCGGCTCTGCTCTTCAG | 38 |
| | | AGCTGCTTTTATGATA | |
| ECK120051383 | rrlB-rrfBrrsB- | caaattaagcAGAAGGCCATCCTGACGGATG | 39 |
| | gltT-rrlB-rrfB | GCCTTTTtgcgtttcta | |
| ECK120035134 | yicC | ccgccttcacAAATGCCGCCACTCAAACAGA | 40 |
| | | GCGGCATTTttcttccccg | |
| ECK120010822 | ilvAilvEDAil | tgcctcaattAGCGGCTCATGTAGCCGCTttttct | 41 |
| | vLXG_1G_2 | gcgc | |
| | MEDA | | |
| ECK120010856 | dcuB- | GGCAGCATGCTGCCAGGTGATCCCCCT | 42 |
| | fumBfumB | GGCCACCTCTTTT | |
| ECK120010793 | rnpB | TACGTAAAAACCCGCTTCGGCGGGTTTT | 43 |
| | | TACTTT | |
| ECK120015459 | talB | taatcattctTAGCGTGACCGGGAAGTCGGTC | 44 |
| | | ACGCTAcctcttctga | |
| ECK120051385 | glmY | CTTATTCCATAACAAAGCCGGGTAATTC | 45 |
| | | CCGGCTTTGTTGTATCTGAAC | |
| ECK120010864 | glmUS | AATAAATGGATGCCCTGCGTAAGCGGG | 46 |
| | | GCATTTTTCTTCCT | |
| ECK120010781 | ompC | ATCGAACAAAGGGCCTGCGGGCCCTTT | 47 |
| | | TTTCATTG | |
| BBa_K088008 | glnRS | agaaacagcaaacaatccaaaacgccgcgttcagcggcgttttt | 48 |
| | | tctgcttttct | |
| ECK120010846 | uspA | cgtgttcctgAACGCCCGCATATGCGGGCGT | 49 |
| | | Tttgctttttg | |
| ECK120010840 | cutC | TGCTCGTACCAGGCCCCTGCAATTTCAA | 50 |
| | | CAGGGGCCTTTTTTTATCC | |
| ECK120010868 | ampC | AATTCCATCGGGTCCGAATTTTCGGACC | 51 |
| | | TTTTCTCCGC | |
| ECK120010853 | htpG | ttcctgatgtAATGCCGGATGACCTTCGTGTC | 52 |
| | | ATCCGGCATTtttcttttca | |
| pheA_2 | pheA | ATGGGAGGCGTTTCGTCGTGTGAAACA | 53 |
| | | GAATGCGAAGACGAACAATAAAGGCCT | |
| | | CCCAAATCGGGGGGCCTTTTTT | |
| ECK125108723 | mgrR | TAGCGTAAAAGCAAAACACAAATCTAT | 54 |
| | | CCATGCAAGCATTCACCGCCGGTTTACT | |
| | | GGCGGTTTTTTTTCGCCGTCATA | |
| ECK120034950 | sspAB | GTGAAGTAATACAAAACAGGCCCAGGC | 55 |
| | | GGCCTGTTTTGTCTTTTTAATG | |
| ECK120010825 | rnb | ATCTCCTTTCACGGCCCATTCCTCATGG | 56 |
| | | ATGGGCCGTTTATTTCCCC | |
| ECK120030221 | relBE-hokD | CCCGCACTTAACCCGCTTCGGCGGGTTT | 57 |
| | | TTGTTTTT | |
| ECK120015454 | glnS | caaacaatccAAAACGCCGCGTTCAGCGGCG | 58 |
| | | TTTTttctgctttt | |
| ECK120010865 | rpoDrpsU- | ctctgcacaaACGCCACCTTTTCGGTGGCGtttt | 59 |
| | dnaG-rpoD | ttatcg | |
| ECK120010806 | glnA | GATGGCTCCGATGGATAACCAGCGCCG | 60 |
| | | CTTAAGTCAGGAA | |
| ECK120020522 | accBC | agcgtcaaaaGGCCGGATTTTCCGGCCttttttatt | 61 |
| | | a | |
| ECK120010833 | fliAZYfliY | GCATAATGACAAAAAAGGGCGCTTTCA | 62 |
| | | CTAGCGCCTTTTTTATTTACGCGT | |
| ECK120029341 | aroH | tctgaatgcgTGCCCATTCCTGACGGAATGG | 63 |
| | | GC Atttctgcgca | |
| BBa_B0052 | rrnC | agaaatcatccttagcgaaagctaaggattttttttatctg | 64 |
| BBa_B0021 | LuxICDABE | aaataataaaaaagccggattaataatctggctttttatattctct | 65 |
| | G reversed | | |
| ECK120011170 | fldA-uof- | AACGAGAAAAGCCAACCTGCGGGTTGG | 66 |
| | furfuruof-fur | CTTTTTTATGCA | |
| ECK120051408 | fadD-sroD | GTCAGTCGTCAGACGCCGGTTAATCCG | 67 |
| | | GCGTTTTTTTTGACGCCCAC | |
| ECK120034551 | gpt | ctaatcttttCAACGCCTGGCACTGCCGGGCG | 68 |
| | | TTGTTCTTTTTaacttcaggc | |
| ECK120035136 | tisB | aagtcgcaccAAAGGGGAGCGGGAAACCGC | 69 |
| | | TCCCCTTTtatatttagc | |
| BBa_B0057 | BBa_B0057 | cagaaatcatccttagcgaaagctaaggattttttttatctg | 70 |
| ECK120029529 | dusB-fis | tgcttgattaAAAAGGCGCTACTCGGCATGG | 71 |
| | | GGAAGCGCCTTTTttataggtgt | |
| ECK120051400 | ivbL-ilvBN | TGAACAACATCGCGCTTATCGTTAAGG | 72 |
| | | TAAGCGCGTATTTTTTTTACCCGCCAG | |
| ECK120020525 | metY | TTATATAAAGCCCCGATTTATCGGGGTT | 73 |
| | | TTTTGTTA | |
| ECK120010851 | rlmE-ftsH | ATTTGTACCGAAAACCCCGGGGCGTGC | 74 |
| | | TCCGGGGTTTTTTCTTATCAA | |
| ECK120034436 | argP | atcaaataatGCCTGATAGCACATATCAGGCg | 75 |
| | | ttgtcctca | |
| ECK120010852 | hslVU | CATTGTTTGATGGGGCTGAAAGGCCCC | 76 |
| | | ATTTTTATTGG | |
| ECK120048898 | fadR | tttaaagagcAAACCCCTCAAACGAGGGGTT | 77 |
| | | Ttttgttgttt | |
| ECK120051404 | mtlADR | cctctacctgCTTCGGCCGATAAAGCCGACGa | 78 |
| | | taatactcc | |
| ECK120021270 | infC-rpmI- | GCCAGTTGAAAGAGGGAGCTAGTCTCC | 79 |
| | rplTrplTrpmI- | CTCTTTTCGTTTC | |
| | rplT | | |
| ECK120030802 | 1drD | GCCCGGACCAGGCCGCAGGGGGGAAA | 80 |
| | | CTCTGCGGCCTTTTTCGTTCTTACT | |
| ECK120010857 | focA- | TGTAATTAGATTTGACTGAAATCGTACA | 81 |
| | pflBpflB | GTAAAAAGCGTACAATAAAGGCTCCAC | |
| | | GAAAGTGGGGCCTTTTTTAGCGCGA | |
| ECK125095454 | tff-rpsB-tsf | atcaaaaaggAGCCGCCTGAGGGCGGCTtcttttt | 82 |
| | | gtg | |
| ECK120020622 | rpsO | CGAGTTTCAGAAAAGGGGGCCTGAGTG | 83 |
| | | GCCCCTTTTTTCAAGCTGAC | |
| ECK120010874 | rpsJ- | TACGAATAAACGGCTCAGAAATGAGCC | 84 |
| | rplCDWB- | GTTTATTTTTTC | |
| | rpsS-rplV- | | |
| | rpsC-rplP- | | |
| | rpmC-rpsQ | | |
| ECK120010783 | mdoGH | acgagccaatAAAAATACCGGCGTTATGCCG | 85 |
| | | GTATTTTTttacgaaaga | |
| ECK120030671 | ileS-lspA- | aagttatgcgAAAATGCCGGTCTTGTTACCG | 86 |
| | fkpB- | GCATTTTttatggagaa | |
| | ispHlspA- | | |
| | fkpB- | | |
| | ispHribF-ileS- | | |
| | lspA-fkpB- | | |
| | ispH | | |
| ECK120010796 | csrB | GAAACGAACCGGGAGCGCTGTGAATAC | 87 |
| | | AGTGCTCCCTTTTTTTATT | |
| ECK125095211 | micM | ctctttgacgGGCCAATAGCGATATTGGCCAT | 88 |
| | | TTTTTTagcgcaacat | |
| ECK120010832 | lolB-ispE- | GGCTCAAAGACCCGCTGCGGCGGGTTT | 89 |
| | prsprs | TTTTGTCT | |
| ECK120030218 | rseX | tattttttggGCCGGCATGATGCCGGCttttttttat | 90 |
| rpsO_pnp | rpsO-pnp | AAAAGGGGCCTGAGTGGCCCCTTTTTTC | 91 |
| | | A | |
| ECK120029531 | tyrB | atgcaggaaaGCAGGCTGGAGCTACCCAGCC | 92 |
| | | TGCagtgaaatta | |
| ECK120020528 | glyQS | GTTATTAAATAGCCTGCCATCTGGCAG | 93 |
| | | GCTTTTTTTATCG | |
| BBa_B0061 | yciA/tonA | aagtcaaaagcctccggtcggaggcttttgacttt | 94 |
| ECK120030798 | tatABCD | agaataaattCAACCGCCCGTCAGGGCGGTT | 95 |
| | | Gtcatatggag | |
| ECK120015460 | pheP | caaccatccgAAACCGCTCTCATCCATTCGA | 96 |
| | | TGAGAGCGGTTTttttaattac | |
| frd_ampC | frd-ampC | CGGCCCGCCTATGGCGGGCCGTTTTGTA | 97 |
| | | T | |
| ECK120051403 | mtlADR | gcctttcaaaAGTAAGCAACGTCTGCTTACTg | 98 |
| | | cccctctac | |
| ECK120010790 | cpxP | tccctgtcttCCCCCACATGCTGTGGGGGtttttttt | 99 |
| | | at | |
| ECK120033262 | glgS | GCACTGATATAACGGGCCTGATGGCCC | 100 |
| | | GTTTTAGTGTTTG | |
| ECK120010831 | aldB | GGTATTCATTGCCTGATGCGACGCTTAC | 101 |
| | | GCGTCTTATCATGCCTACGGGAACCTG | |
| | | A | |
| ECK120033265 | cspA | cacagaatctAAGATCCCTGCCATTTGGCGG | 102 |
| | | GGATTTTtttatttgtt | |
| ECK120010826 | glnK-amtB | caagcactgcAAAAAACAGCCGGACGGTTTT | 103 |
| | | CACCTCCGGCTATTTTTTtaattgtgat | |
| nusA_infB | nusA-infB | CCCCGATTTATCGGGGTTTTTTGTTATC | 104 |
| | | TGACTACAGAATAACTGGGCTTTAGGC | |
| | | CCTTTTTTT | |
| ECK120035132 | pstSCA | CTCACCCTAACCCTCTCCCCAGAGGGG | 105 |
| | | CGAGGGGACCGACCGA | |
| ECK120010834 | rpsU | tgtagttgtaAGGCCGTGCTTCCGAAAGGAAT | 106 |
| | | GCGCGGCTTattttcgttt | |
| ECK120010812 | gadAXgadX | GGGAAGAGGATAGTCTGCCGTCTCCAG | 107 |
| | | ACTAATAAACCGTT | |
| ECK120030672 | dapD | AGTATGCACACGGGCAGCACGACGCTG | 108 |
| | | CCCGATTTTTTTGCA | |
| ECK120029599 | aroG | tagcaacaaaAAAGCCGACTCACTTGCAGTC | 109 |
| | | GGCTTTctcattttaa | |
| trp | trp | AGCCCGCCTAATGAGCGGGCTTTTTTTT | 110 |
| ECK120010873 | metY-rimP- | GTTGCCATTTGCCCTCCGCTGCGGCGGG | 111 |
| | nusA-infB- | GGGCTTTTAACCGGG | |
| | rbfA-truB- | | |
| | rpsO- | | |
| | pnppnprpsO- | | |
| | pnp | | |
| ECK120010841 | rpoH | AAGCAGAGAACCCTGGATGAGAGTCCG | 112 |
| | | GGGTTTTTGTTTTT | |
| BBa_B0051 | yciA/tonA | aaagtcaaaagcctccgaccggaggctttigactt | 113 |
| ECK120010805 | gadY | ctatcctcttCCCGGTCCCCTATGCCGGGtttttttt | 114 |
| | | at | |
| ECK125109867 | arcZ | gtattcgcgcACCCCGGTCTAGCCGGGGTCA | 115 |
| | | TTTTTTagtggctttt | |
| ECK120030673 | dapB | tattgattatAAAGGGCTTTAATTTTTGGCCCT | 116 |
| | | TTtatttttggt | |
| ECK120034956 | rhoL-rho | cgtctgcgtaTGGAACGTGGTAACGGTTCTAc | 117 |
| | | tgaagattt | |
| ECK120010780 | glyA | TGATCATCAAGGCTTCCTTCGGGAAGC | 118 |
| | | CTTTCTACGTTA | |
| ECK120029530 | guaBA | ATGAATGAACAAAACCCTCTGTTACTA | 119 |
| | | CAGAGGGTTTTTTATCTTCAA | |
| ECK120010784 | bamCdapA- | GTAAAAATACAGGGCTGGAATCATCCG | 120 |
| | bamC | GCCCTTTTTTCTGAT | |
| ECK120010798 | bglGFB | CTAAGCGGGCAAAACCTGAAAAAAATT | 121 |
| | | GCTTGATTCACGTCAGGCCGTTTTTTTC | |
| | | AGGTTTTTTTTTGGAGT | |
| ECK120010830 | tgt-yajC | gctgttcagaTCACTGGTGCGCGCGGAGTCG | 122 |
| | | CCGCCAGTGAgcaaacgctg | |
| ECK125095455 | greA | GTGAATTGTAGCTGACCTGGGACTTGT | 123 |
| | | ACCCGGGTCGGTATTTTTTTGCTTCTGG | |
| | | TCCCGG | |
| ECK120010800 | gntKUgntRKU | AGTTTGTTCGCCCGGTAGTTGTGACGCT | 124 |
| | | ACCGGGTTCTTTTCGA | |
| ECK120048901 | cstA | ttagtgcccaGGGTTCCCTCTCACCCTAACCC | 125 |
| | | TCTCCCCGGTGGGGCGAGGGGACTgaccg | |
| | | agcgc | |
| ECK120034434 | feaB | gctcataagtAAAAAACGGCACCTGGTGCCG | 126 |
| | | TTTTTTtgtctgaaac | |
| ECK120010809 | malZ | ccgtttaacaCGTTCTGGATGAAATCCATATC | 127 |
| | | Gcgatagcgca | |
| ECK120048902 | cysK | gcgtaaaaaaGCACCTTTTTAGGTGCttttttgtgg | 128 |
| ECK120035138 | cyaR | gaaccacctcCTTAGCCTGTGTAATCTCCCTT | 129 |
| | | ACACGGGCTTATTTTTTacgegtaata | |
| ECK120010821 | hupB | gaagttcaagGGCGCATCTACTGATGTGCCtttt | 130 |
| | | ttattt | |
| ECK120030219 | gcvPgcvTHP | ACTATTTTCTAAAGGCGCTTCGGCGCCT | 131 |
| | | TTTTAGTCAGAT | |
| ECK120026395 | acrEF | gataaatcagAAACATAAAGGCGCTTTCGGG | 132 |
| | | TGCCTTTATTATTTccagtgaaac | |
| ECK125109871 | metZWV | ccataaaaaaGCGCCATTCAGCGCCTTTTTAt | 133 |
| | | catcccctt | |
| ECK120010827 | adiA | TTCCGCTGAAGGCGTAATTGTTTAAATA | 134 |
| | | ACATTACGCCGCCTGGCCTT | |
| ECK120010849 | hypABCDEh | atgaccttttGCACCGCTTTGCGGTGCTttcctgga | 135 |
| | ypBCDE | ag | |
| ECK120029975 | trpR | taagacgtggCGCATCAGGCATCGTGCACCG | 136 |
| | | AATGCCGGATGCGgcgtgaacgc | |
| ECK120010791 | malEFG | ACTGTTATTCGGCGCTCCACGGAGCGC | 137 |
| | | CTTTTTTTCT | |
| ECK120010819 | hupA | agttttaacgAAGGGGTGGTTTCACCCCTTttgt | 138 |
| | | ctttct | |
| ECK120048897 | malI | CCGGCTCATTGCAGCGAAATAATCCTCT | 139 |
| | | CTTTATCTGCTATACCTGGT | |
| ECK120027937 | bolA | gattttatgaAAAACGGCCTGCGGGCCGTTTT | 140 |
| | | gttttgtctg | |
| ECK120010802 | gntP | AAGGACACCAGAGCCTGCCAATGGCAG | 141 |
| | | GCTCAGACTGATGA | |
| tonB | tonB | AGTCAAAAGCCTCCGACCGGAGGCTTT | 142 |
| | | TGACTATTACT | |
| ECK120010803 | edaedd-eda | AAAGTCAAAATGCCCGATCGAGGATCG | 143 |
| | | GGCATTTTTGTAGC | |
| ECK120010836 | ryjAsoxR | aagatgaacaAAACTAAAGCGCCACAAGGG | 144 |
| | | CGCTTTAGTTTgttttccggt | |
| ECK120029528 | aroP | ATCTCTCTACGCCCTCACCCGTACAGGG | 145 |
| | | TGAGGGCAATAATCTTT | |
| pheST | pheST | AGCCTCCCAGTGGAGGCTTTTTTTGT | 146 |
| his | his | AAAGCCCCCGGAAGATCACCTTCCGGG | 147 |
| | | GGCTTT | |
| ECK120010813 | exbBDexbD | CACAATGATGCCCGGTTGCTTTTCACAA | 148 |
| | | CCGGGCATTTTTTTAAC | |
| ECK120015957 | treR | CCTGTCCTGATCGTTTCCTGAACGATAA | 149 |
| | | ATTGTGA | |
| ECK120010824 | ilvY | GCGCGGGTAGGCCTGATAAGCGAAGCG | 150 |
| | | CTATCAGGCATTTTTCCCTA | |
| BBa_B0053 | his | tccggcaaaaaaacgggcaaggtgtcaccaccctgccctttttct | 151 |
| | | ttaaaaccgaaaagattacttcgcgtt | |
| ECK120033127 | sdaA | tacttcttacTCGCCCATCTGCAACGGATGGG | 152 |
| | | CGAatttataccc | |
| ECK120015448 | amyA | gaggtgatttAAATTCATCCCCGGCGGCAAG | 153 |
| | | CCGGGGAGATTTcattacggca | |
| ECK120010792 | tonB | gtaagcagaaAGTCAAAAGCCTCCGACCGG | 154 |
| | | AGGCTTTTGACTattactcaac | |
| ECK120015446 | gcd | CAGATTGCTGACAACGTGCGCGTTGTTC | 155 |
| | | ATGCCGGA | |
| ECK120010843 | tsx | CTGATTATGAAAATGCCGGGATTTATTC | 156 |
| | | CCGGCATTTCTGATTGTTA | |
| ECK120010848 | malS | cagccctaatCAGCGTTGCAGGATAAAGCAC | 157 |
| | | CGCTCactcttcaac | |
| ECK120010816 | glpEGRglpG | ACGGCTTCCCACGTCAGACCAAAACGC | 158 |
| | RglpR | GCCAGGTATTTGCGTAGCCGATCCGCG | |
| | | TCATTGACGCTGGCTTTGCCCTGGCGCG | |
| | | AAACGTCAAAAAGCTG | |
| ECK120015439 | nudB-yebC- | ACTGCGTTAAGTTATACCGCCTCGGTCA | 159 |
| | ruvCruvCyeb | GTTCCGGCTGAGGCGTTTCCACTCCCTC | |
| | C-ruvC | CGCA | |
| ECK120010845 | cvpA-purF- | AACTCCGCTGTTGCCCTGTTTCAGGGCA | 160 |
| | ubiXubiX | ATTTTGCAACC | |
| ECK120010787 | tyrR | agtaagcgcgAATATGCCTGATGGTGCAACA | 161 |
| | | CCATCAGGCATATTaaattatgct | |
| ECK120051405 | mtlADR | aatcgcgttaCGGGGAGGAAGTTTTTTCAGA | 162 |
| | | TACTCCCggaacgcctg | |
| ECK120048899 | ansB | TAATCGCCTCGCCCCGGTATCGTGCCGG | 163 |
| | | GGCTTTTTCACTT | |
| ECK120030220 | cynTS | ttacccgcaaAGTGCGCTGCTGCTTAGCAGC | 164 |
| | | GCACTgcttggtggg | |
| BBa_B0060 | pBR322 | aaaatcaaaggatcttcttgagatccttttttt | 165 |
| ECK120010807 | bglGFB | TTAAAGCACCTTAATTATCGTCGCATTC | 166 |
| | | AGAACAGTCTGGATGCGATGCGTTAAT | |
| | | TCTTTCTTT | |
| ECK120026315 | dnaTC- | CTGCCCTTTTCCCTGCTCCTGGACGGTT | 167 |
| | yjjAyjjB- | TTACCCCT | |
| | dnaTC-yjjA | | |
| ECK120033264 | ivbL-ilvBN | CCCAATGACTACTTCCATGCTCAACGCA | 168 |
| | | AAACTACTACCAACTGCGCCATCCGCC | |
| | | GCAGTGGTCGTCGTGCGTGTGGTGGTG | |
| | | GTCGTCGGCAATGCGCCGTAGGGACTG | |
| | | GAACAACACACGATTCCAAAACCCCGC | |
| | | CGGCGCAAACCGGGCGGGGTTTTTCGT | |
| | | TTAAG | |
| ECK120051406 | mtlADR | ccgtattaccCCGCGCCGGGAATGCGCGGccg | 169 |
| | | ccaattt | |
| ECK125122040 | yciEyciGFE | TACCATGTCCTTATTGACCCCGTATATT | 170 |
| | | ACGGGGTCGTTTTTGTGCGGAAT | |
| ECK120010844 | nuoABCEFG | AGTCAGAAAGCCGCCGACATGCTCGGC | 171 |
| | HIJKLMNnu | GGTTTTTCTGAA | |
| | oMN | | |
| ECK120010779 | aroF- | ctgatgaaaaGGTGCCGGATGATGTGAATCA | 172 |
| | tyrApheLA | TCCGGCACtggattatta | |
| ECK120030670 | mreB | TAATCGGATGCAGGCAGGGGAAGTGTC | 173 |
| | | TGTTTACCCTGCCTGGTCTGATACG | |
| ECK120015455 | malT | gtgtaagtttAGCCGGATAACGCGCCAGATC | 174 |
| | | CGGCTtacatctctg | |
| ECK120015450 | bglX | ATGCTTTAGTAAGGGCGCGACGTTTGC | 175 |
| | | GCCCTTTGTAGGCCGG | |
| ECK120015456 | epd-pgkpgk | AAAAAATCACAGGGCAGGGAAACCTGC | 176 |
| | | CCTTGTTTCAGCG | |
| ECK120010862 | putP | gctccgccgtCACGGTTGCAGGAAAGCTAAG | 177 |
| | | GGACTTAGCCTGCGGCGGTTTTGTttggctt | |
| | | cag | |
| ECK125108943 | selD- | CTGAAATATCCAGCGGATCAAGAAAAT | 178 |
| | topBydjA- | TCGTTGGATATTTTTT | |
| | selD-topB | | |
| ECK120035135 | dacD | CTTTCTTTTGCAGCAGACTGGCAGGAGT | 179 |
| | | GCGAGTCTGCTCGCATAATCA | |
| ECK120015449 | glpFKX | CGATTGAGCCTTCCAGTCCTTCGGGACT | 180 |
| | | GGAATTTTTTTGTT | |
| ECK120010842 | hyaABCDEF | ccgacgtaaaAAGACGGTAAGTATCGCTTTC | 181 |
| | | AGTCTTatgaatatcg | |
| ECK125095210 | pldA | gtttctcgcgCAGGCGCTGAAAATAGCGCCT | 182 |
| | | Gtttttatttc | |
| ECK120034954 | rhoL-rho | ggctggaaaaCCTGGCTCGTATGCGTAAGCA | 183 |
| | | GGacattatttt | |
| ECK120010788 | cynTSXlacY | gaacttgtagGCCTGATAAGCGCAGCGTATC | 184 |
| | AlacZYA | AGGCaatttttata | |
| ECK120010860 | araBAD | TCAAACGAAACCAGGCTATACTCAAGC | 185 |
| | | CTGGTTTTTTGATGG | |
| ECK120010801 | asr | cagtaatgctGGCGCGCCCCCTCGCGCCtgaaaa | 186 |
| | | ttac | |
| ECK120015458 | aspS | CTGAGTGAACTCCCATGAGCATAGATA | 187 |
| | | ACTATGTGAATGGGATGAGCGAAGG | |
| ECK120034948 | ecnB | aataagcaatAACGGTACGACAGCTGTGTCG | 188 |
| | | TGCCGTttgttttttc | |
| ECK120015453 | katE | aaacacgtagGCCTGATAAGCGAAGCGCATC | 189 |
| | | AGGCagttttgcgt | |
| ECK125109037 | otsAotsBA | GGCAATAACTCTTTTCGCCGAGCAGGA | 190 |
| | | TGCTCGCGAAAAGAAACTGTGATT | |
| ECK125109868 | gadWgadXW | AGCAGGAAAGAGTAAGGCTGAACCTTC | 191 |
| | | ATGTTCAACCTTACTCTCATTTAC | |
| ECK120010828 | cysB | tgcgttatttTCGGCACCTTTTATGTAGCGAA | 192 |
| | | GGTGCCGGaatatattct | |
| ECK120010814 | aceEF | tgatgtaagtAAAAGAGCCGGCCCAACGGCC | 193 |
| | | GGCTTTTTTctggtaatct | |
| ECK120034949 | yjaZ | caaaataacaAAACCCACCTTAAGGTGGGTT | 194 |
| | | Tcgccagagaa | |
| ECK125108944 | selD-topB | ATATTCTGAAATATCCAGCGGATCAAG | 195 |
| | | AAAATTCGTTGGATAT | |
| ECK120010820 | hupB | ctaagcgttgTCCCCAGTGGGGAtgtgacgaag | 196 |
| ECK120010829 | phoH | gttatcggtgCAGAGCCCGGGCGAACCGGGC | 197 |
| | | TTTGttttgggtgt | |
| ECK120010837 | fliDST | cgaataatccGATTACGGCTACGCTTCTAAT | 198 |
| | | GTTCCCCTTGAATGGAGTCGAAGAATG | |
| | | CGTAATCccacgctgtt | |
| BBa_B0011 | luxICDABEG | agagaatataaaaagccagattattaatccggcttttttattattt | 199 |
| ECK120015443 | rpiA | AATGATCTGACGGGGGAACCTCCCCCG | 200 |
| | | TTAAAAAAAT | |
| ECK120020526 | metY | CAGAATAACTGGGCTTTAGGCCCTTTTT | 201 |
| | | TTATG | |
| trp_t | trp t | GCCGCCAGTTCCGCTGGCGGCATTTT | 202 |
| ECK120010839 | endA | ttaacccctaCCCCACGCGTACAACCGCGTG | 203 |
| | | GGGagacgacgcg | |
| ECK120030803 | kdsAychQA- | cttgaaaaatAAAGTATTAGCGTTCTGCGTTA | 204 |
| | kdsA | AGACTTTtttcatgggt | |
| ECK120051407 | hscBA-fdx- | AATAATTTCCGCGTCATGCTTCACGCCG | 205 |
| | iscX | CAGATGCGTTGGCTGCG | |
| ECK120026312 | dnaTCyjjB- | TTCTGTGCTGTGCCATTGCCGCCAGCGC | 206 |
| | dnaTC | ATTAATTTCCA | |
| ECK120010866 | fldA-uof- | TAAGAAAAGCGAGAGTTACAGCTCTCA | 207 |
| | furfuruof-fur | CTTATTTGTT | |
| tetA | tetA | CCCTCTTGATAACCCAAGAGGGCATTTT | 208 |
| | | TTA | |
| rplKAJL_rpoBC | rplKAJL- | GGCGTGAGATTGGAATACAATTTCGCG | 209 |
| | rpoBC | CCTTTTGTT | |
| ECK120015526 | ruvAB | CGAACCGTAGGTCGGATAAGGCGCTCG | 210 |
| | | CGCCGCATCCGACAAATGTGTTC | |
| ECK120030801 | trmA | AGTAAAACCCATGCCGGATGCGCCAGC | 211 |
| | | ATCCGGCATAATACCGATTAC | |
| ECK120015445 | agp | ataacagaaaACTCCCCCGCGAGAAGCGGGG | 212 |
| | | GAGTcgctggttaa | |
| ECK120030800 | psrD | aattattggcAAAAGGCAACCACAGGCTGCC | 213 |
| | | TTTTtctttgactc | |
| ECK120010810 | malZ | cagccactgcTCTGACCACAAGTAATTGTTC | 214 |
| | | AGAttgataaaac | |
| ECK120011168 | gntRgntRKU | CCGGCTGGACAATGTTACCGATAACAG | 215 |
| | | TTACCCGTAACATTTTTAATTCTT | |
| ECK120015451 | hycABCDEF | ATGCTAAATTGCCCGATGCGCTGCGCTT | 216 |
| | GHI | ATCGGGCCTTCATGGTT | |
| ECK120048900 | cdh | aggaaagtaaGTGCCGGATATGAAATCCGGC | 217 |
| | | ACctgtcagact | |
| ECK120033738 | rpsP-rimM- | TTTAATATGACACCGGACTCCGTTCCTC | 218 |
| | trmD-rplS | GATGGGGTCCGGTTGTTTTATTCAC | |
| ECK120010847 | malS | aacagtaactTTTCCGGCTTCCCGTTCGTCAG | 219 |
| | | TACCTCGGGAAGCCGCCAAccaggataaa | |
| orfL | orfL | CCCTCTTGGGTTATCAAGAGGGTCATTA | 220 |
| | | TATTT | |
| ECK125109869 | rph-pyrE | TAAAGAAACTCGCCGGATGAAAAGTCA | 221 |
| | | TCCGGCGTCATATTACT | |
| ECK120010817 | cmr | gggtaaaaaaATGCCTGACTGCTTTGTGCGA | 222 |
| | | TCAGGCATictcgaatta | |
| ECK120020527 | yhdT-panF- | ccgattatttACGCAAATTTGCGTgccaaaattt | 223 |
| | prmA | | |
| ECK120035131 | pstS | AAAACTCCAGGCCGGGTACGGTGTTTT | 224 |
| | | ACGCCGCATCCGGCATTACAAAAT | |
| ECK120010838 | endA | acgcgtacaaCCGCGTGGGGAGACGACGCG | 225 |
| | | Gatttttaact | |
| ECK120027917 | yacC-speED | GTTTAACGGCTCTGGCGGAGCTCCCAG | 226 |
| | | GCTCCGCCAGATTTATTTACT | |
| ECK120010785 | rpiB | agaaattgagATTCATCCACTACTTGCATGG | 227 |
| | | ATGAGTaatgattaat | |
| ECK120029978 | yjbE | taaagtatgtATCCCCAAAATAATTCGAGTCA | 228 |
| | | TTGCATCTGTGGCTAGAAGTATGAAGG | |
| | | GAttaaccataa | |
| ECK120010858- | aspA | acaagaaaaaAGGCACGTCATCTGACGTGCC | 229 |
| R | | ttttttattt | |
| ECK120026481- | arcA | aaggttgaaaAATAAAAACGGCGCTAAAAA | 230 |
| R | | GCGCCGTTTTTTTTgacggtggta | |
| ECK120010835- | cysDNC | cgcaaataacCAGGAGATAAAACCGACCAC | 231 |
| R | | GGCACCAGGCAGTGACCATGTGGTTTC | |
| | | TTCAtcctcagtaa | |
| ECK120015457- | ruvAB | ctctggtagtCCTGGTAAGACGCGAACAGCG | 232 |
| R | | TCGCATCAGGcatattgcca | |
| ECK120010836- | ryjAsoxR | ACCGGAAAACAAACTAAAGCGCCCTTG | 233 |
| R | | TGGCGCTTTAGTTTTGTTCATCTT | |
| ECK120026314- | valS | tgtaagagatAAAAAAGGCCGGAGCATGCTC | 234 |
| R | | CGGCCttcgttttca | |
| ECK120010812- | gadAXgadX | aacggtttatTAGTCTGGAGACGGCAGACTAt | 235 |
| R | | cctcttccc | |
| ECK120010832- | lolB-ispE- | agacaaaaaaACCCGCCGCAGCGGGtctttgagcc | 236 |
| R | prsprs | | |
| ECK120011170- | fldA-uof- | tgcataaaaaAGCCAACCCGCAGGTTGGCttttc | 237 |
| R | furfuruof-fur | tcgtt | |
| ECK120010841- | rpoH | aaaaacaaaaACCCCGGACTCTCATCCAGGG | 238 |
| R | | ttctctgctt | |
| ECK120026315- | dnaTC- | aggggtaaaaCCGTCCAGGAGCAGGGaaaaggg | 239 |
| R | yjjAyjjB- | cag | |
| | dnaTC-yjjA | | |
| ECK120020622- | rpsO | gtcagcttgaAAAAAGGGGCCACTCAGGCCC | 240 |
| R | | CCTTTTctgaaactcg | |
| ECK120015439- | nudB-yebC- | lgcggagggaGTGGAAACGCCTCAGCCGGA | 24 |
| R | ruvCruvCyeb | ACTGACCGAGGCGGTATAACttaacgcagt | |
| | C-ruvC | | |
| ECK120010825- | rnb | ggggaaataaACGGCCCATCCATGAGGAAT | 242 |
| R | | GGGCCGTgaaaggagat | |
| ECK120010782- | fhuE | tggggagactAAGGCAGCCAGATGGCTGCCT | 243 |
| R | | Tttttacaggt | |
| ECK120017009- | ihfApheMST- | agtgaaaagaAAAAAGGCCGCAGAGCGGCC | 244 |
| R | ihfA | TTTTtagttagatc | |
| ECK120010806- | glnA | ttcctgacttAAGCGGCGCTGGTTATCCATcgg | 245 |
| R | | agccatc | |
| ECK120015446- | gcd | tccggcatgaACAACGCGCACGTTGTcagcaatct | 246 |
| R | | g | |
| ECK120010804- | aes | aaaaatatgaATATATTCCGGCGCTTAATGCC | 247 |
| R | | ACGCCGGAACATATcgaaatgatg | |
| ECK120029530- | guaBA | ttgaagataaAAAACCCTCTGTAGTAACAGA | 248 |
| R | | GGGTTTTgttcattcat | |
| ECK120010813- | exbBDexbD | gttaaaaaaaTGCCCGGTTGTGAAAAGCAAC | 249 |
| R | | CGGGcatcattgtg | |
| ECK120010793- | rnpB | aaagtaaaaaCCCGCCGAAGCGGGtttttacgta | 250 |
| R | | | |
| ECK120010856- | dcuB- | aaaagaggtgGCCAGGGGGATCACCTGGCAg | 251 |
| R | fumBfumB | catgctgcc | |
| ECK120015458- | aspS | ccttcgctcaTCCCATTCACATAGTTATCTAT | 252 |
| R | | GCTCATGGGAgttcactcag | |
| ECK120015447- | glk | tttaaaagatTATCGGGAGAGTTACCTCCCGA | 253 |
| R | | TAtaaaaggaag | |
| ECK120010861- | frdABCD | tccatacaaaACGGCCCGCCATAGGCGGGCC | 254 |
| R | | Ggatttacatt | |
| ECK120010863- | atpBEFHAG | cgcaaaaaaaAGCCAGCCTGTTTCCAGACTG | 255 |
| R | DCatpIBEFH | GCttttgtgctt | |
| | AGDC | | |
| ECK120010867- | xapAB | tatcataaaaGCAGCTCTGAAGAGCAGAGCC | 256 |
| R | | GCgaatcctttt | |
| ECK120010860- | araBAD | ccatcaaaaaACCAGGCTTGAGTATAGCCTG | 257 |
| R | | Gtttcgtttga | |
| ECK120010794- | rnpB | aaagcaaaaaCCCGCCGAAGCGGGtttttacgta | 258 |
| R | | | |
| ECK120034435- | secG-leuU | ggaccaaaacGAAAAAAGACGCTTTTCAGC | 259 |
| R | | GTCTCTTTTCTGGAATTtggtaccgag | |
| ECK120010789- | lacY AlacZYA | gggtcaaagaGGCATGATGCGACGCTTGTTC | 260 |
| R | | CTGCGCTTTGTTCATGCCGgatgcggcta | |
| ECK120010803- | edaedd-eda | gctacaaaaaTGCCCGATCCTCGATCGGGCAt | 261 |
| R | | tttgacttt | |
| ECK120010864- | glmUS | aggaagaaaaATGCCCCGCTTACGCAGGGC | 262 |
| R | | Atccatttatt | |
| ECK120015526- | ruvAB | gaacacatttGTCGGATGCGGCGCGAGCGCC | 263 |
| R | | TTATCCGACctacggttcg | |
| ECK120010802- | gntP | tcatcagtctGAGCCTGCCATTGGCAGGCTCtg | 264 |
| R | | gtgtcctt | |
| ECK120010874- | rpsJ- | gaaaaaataaACGGCTCATTTCTGAGCCGtttat | 265 |
| R | rplCDWB- | tcgta | |
| | rpsS-rplV- | | |
| | rpsC-rplP- | | |
| | rpmC-rpsQ | | |
| ECK120010843- | tsx | taacaatcagAAATGCCGGGAATAAATCCCG | 266 |
| R | | GCATTTtcataatcag | |
| ECK120048897- | malI | accaggtataGCAGATAAAGAGAGGATTATT | 267 |
| R | | TCGCTGCaatgagccgg | |
| ECK120010851- | rlmE-ftsH | ttgataagaaAAAACCCCGGAGCACGCCCCG | 268 |
| R | | GGGTTTTcggtacaaat | |
| ECK120021270- | infC-rpmI- | gaaacgaaaaGAGGGAGACTAGCTCCCTCTtt | 269 |
| R | rplTrplTrpmI- | caactggc | |
| | rplT | | |
| ECK120011168- | gntRgntRKU | aagaattaaaAATGTTACGGGTAACTGTTAT | 270 |
| R | | CGGTAACATTgtccagccgg | |
| ECK120015170- | rplM-rpsI | ttttagctatAAAAAAACCCGCCGAAGCGGGT | 271 |
| R | | TTTTTcgaaaattgt | |
| ECK120010781- | ompC | caatgaaaaaAGGGCCCGCAGGCCCtttgttcgat | 272 |
| R | | | |
| ECK120010815- | gadBC | aagcacgaaaAAGGGAGCGATGAATTATCG | 273 |
| R | | CTCCCTTgtcttataac | |
| ECK120010872- | rpmBGyicR- | ataacaaaaaACCCCGCCGGAGCGAGGttttttgtt | 274 |
| R | rpmBG | a | |
| ECK120027917- | yacC-speED | agtaaataaaTCTGGCGGAGCCTGGGAGCTC | 275 |
| R | | CGCCAGAgccgttaaac | |
| ECK120030221- | relBE-hokD | aaaaacaaaaACCCGCCGAAGCGGGTtaagtgcg | 276 |
| R | | gg | |
| ECK120015449- | glpFKX | aacaaaaaaaTTCCAGTCCCGAAGGACTGGA | 277 |
| R | | Aggctcaatcg | |
| ECK120030802- | ldrD | agtaagaacgAAAAAGGCCGCAGAGTTTCCC | 278 |
| R | | CCCTGCGGCCtggtccgggc | |
| ECK120021269- | pheMpheMST- | tacaaaaaaaGCCTCCACTGGGAGGCtttcaggcg | 279 |
| R | ihfArpIT- | c | |
| | pheM | | |
| ECK120030672- | dapD | tgcaaaaaaaTCGGGCAGCGTCGTGCTGCCC | 280 |
| R | | Gtgtgcatact | |
| ECK120010857- | focA- | tcgcgctaaaAAAGGCCCCACTTTCGTGGAG | 281 |
| R | pflBpflB | CCTTTATTGTACGCTTTTTACTGTACGA | |
| | | TTTCAGTCAAAtctaattaca | |
| ECK120023928- | pros | caatccatgtAAAAAAAGGGCCCTGAAATTC | 282 |
| R | | AGGACCCTTTCtggcatcagc | |
| ECK120015451- | hycABCDEF | aaccatgaagGCCCGATAAGCGCAGCGCATC | 283 |
| R | GHI | GGGCaatttagcat | |
| ECK120010824- | ilvY | tagggaaaaaTGCCTGATAGCGCTTCGCTTA | 284 |
| R | | TCAGGCctacccgcgc | |
| ECK120010844- | nuoABCEFG | ttcagaaaaaCCGCCGAGCATGTCGGCGGcttt | 285 |
| R | HIJKLMNnu | ctgact | |
| | oMN | | |
| ECK120010808- | bglGFB | tataaacaaaAAAACCCGACTTCACCAGTAT | 286 |
| R | | TCTCTGGTTATGTCAGGTTTTgcctgcgaat | |
| ECK120010833- | fliAZYfliY | acgcgtaaatAAAAAAGGCGCTAGTGAAAG | 287 |
| R | | CGCCCTTTTTTgtcattatgc | |
| ECK120010831- | aldB | tcaggttcccGTAGGCATGATAAGACGCGTA | 288 |
| R | | AGCGTCGCATCAGGCaatgaatacc | |
| ECK120035131- | pstS | attttgtaatGCCGGATGCGGCGTAAAACACC | 289 |
| R | | GTACCCGGCctggagtttt | |
| ECK120035132- | pstSCA | tcggtcggtcCCCTCGCCCCTCTGGGGAGAG | 290 |
| R | | GGttagggtgag | |
| ECK120010788- | cynTSXlacY | TATAAAAATTGCCTGATACGCTGCGCTT | 291 |
| R | AlacZYA | ATCAGGCCTACAAGTTC | |
| ECK120020528- | glyQS | cgataaaaaaAGCCTGCCAGATGGCAGGCTat | 292 |
| R | | ttaataac | |
| ECK120010798- | bglGFB | actccaaaaaAAAACCTGAAAAAAACGGCCT | 293 |
| R | | GACGTGAATCAAGCAATTTTTTTCAGGT | |
| | | TTTgcccgcttag | |
| ECK120010796- | csrB | aataaaaaaaGGGAGCACTGTATTCACAGCG | 294 |
| R | | CTCCCggttcgtttc | |
| ECK120010791- | malEFG | agaaaaaaagGCGCTCCGTGGAGCGCCgaataa | 295 |
| R | | cagt | |
| ECK120020526- | metY | cataaaaaaaGGGCCTAAAGCCCagttattctg | 296 |
| R | | | |
| ECK120016882- | rpsT | cataaaaaaaCCCGCTTGCGCGGGctttttcaca | 297 |
| R | | | |
| ECK120035135- | dacD | tgattatgcgAGCAGACTCGCACTCCTGCCA | 298 |
| R | | GTCTGCTGcaaaagaaag | |
| ECK120015456- | epd-pgkpgk | cgctgaaacaAGGGCAGGTTTCCCTGCCCTgt | 299 |
| R | | gatttttt | |
| ECK120030670- | mreB | cgtatcagacCAGGCAGGGTAAACAGACACT | 300 |
| R | | TCCCCTGCCTGcatccgatta | |
| ECK120010800- | gntKUgntRKU | tcgaaaagaaCCCGGTAGCGTCACAACTACC | 301 |
| R | | GGGcgaacaaact | |
| ECK120010855- | osmE | aggccaaaaaAAACCGGCGCAATGGCCGGT | 302 |
| R | | TTccgttgttac | |
| ECK120030801- | trmA | gtaatcggtaTTATGCCGGATGCTGGCGCAT | 303 |
| R | | CCGGCATgggttttact | |
| ECK120010868- | ampC | gcggagaaaaGGTCCGAAAATTCGGACCcgat | 304 |
| R | | ggaatt | |
| ECK120015443- | rpiA | atttttttaaCGGGGGAGGTTCCCCCGtcagatcatt | 305 |
| R | | | |
| ECK120011169- | gntKU | catgactaaaAACAGCAGCAGTAAAACAGAC | 306 |
| R | | CCTACTGCTGTTaaaacaagcg | |
| ECK120033738- | rpsP-rimM- | gtgaataaaaCAACCGGACCCCATCGAGGAA | 307 |
| R | trmD-rplS | CGGAGTCCGGTGtcatattaaa | |
| ECK120010807- | bglGFB | aaagaaagaaTTAACGCATCGCATCCAGACT | 308 |
| R | | GTTCTGAATGCGACGATAATTAAggtgcttt | |
| | | aa | |
| ECK120010779- | aroF- | TAATAATCCAGTGCCGGATGATTCACA | 309 |
| R | tyrApheLA | TCATCCGGCACCTTTTCATCAG | |
| ECK120010780- | glyA | taacgtagaaAGGCTTCCCGAAGGAAGCCttga | 310 |
| R | | tgatca | |
| ECK120020525- | metY | taacaaaaaaCCCCGATAAATCGGGGctttatataa | 311 |
| R | | | |
| ECK120029528- | aroP | aaagattattGCCCTCACCCTGTACGGGTGAG | 312 |
| R | | GGCgtagagagat | |
| ECK120010866- | fldA-uof- | aacaaataagTGAGAGCTGTAACTCTCgcttttctt | 313 |
| R | furfuruof-fur | a | |
| ECK120048899- | ansB | aagtgaaaaaGCCCCGGCACGATACCGGGGC | 314 |
| R | | gaggcgatta | |
| ECK120026312- | dnaTCyjjB- | tggaaattaaTGCGCTGGCGGCAATGGCAcag | 315 |
| R | dnaTC | cacagaa | |
| ECK120010852- | hslVU | ccaataaaaaTGGGGCCTTTCAGCCCCAtcaaac | 316 |
| R | | aatg | |
| ECK120010786- | recA | aaaagcaaaaGGGCCGCAGATGCGACCCttgtg | 317 |
| R | | tatca | |
| L3S2P21 | synthetic | CTCGGTACCAAATTCCAGAAAAGAGGC | 318 |
| | | CTCCCGAAAGGGGGGCCTTTTTTCGTTT | |
| | | TGGTCC | |
| L3S2P56 | synthetic | CTCGGTACCAAATTTTCGAAAAAAGAC | 319 |
| | | GCTGAAAAGCGTCTTTTTTCGTTTTGGT | |
| | | CC | |
| L3S2P51 | synthetic | CTCGGTACCAAAAAAAAAAAAAAAGA | 320 |
| | | CGCTGAAAAGCGTCTTTTTTCGTTTTGG | |
| | | TCC | |
| L3S1P56 | synthetic | TTTTCGAAAAAAGGCCTCCCAAATCGG | 321 |
| | | GGGGCCTTTTTTATTGATAACAAAA | |
| L3S3P41 | synthetic | AAAAAAAAAAAACACCCTAACGGGTGT | 322 |
| | | TTTTTTTTTTTTGGTCTCCC | |
| L3S3P51 | synthetic | AAAAAAAAAAAACACCCTAACGGGTGT | 323 |
| | | TTTTTTGTTTCTGGTCTCCC | |
| L3S1P52 | synthetic | TCTAACTAAAAAGGCCTCCCAAATCGG | 324 |
| | | GGGGCCTTTTTTATTGATAACAAAA | |
| L3S2P41 | synthetic | CTCGGTACCAAAAAAAAAAAAAAAGA | 325 |
| | | CGCTGAAAAGCGTCTTTTTTTTTTTTGG | |
| | | TCC | |
| L3S2P22 | synthetic | CTCGGTACCAAATTCCAGAAAAGAGGC | 326 |
| | | CGCGAAAGCGGCCTTTTTTCGTTTTGGT | |
| | | CC | |
| L3S2P11 | synthetic | CTCGGTACCAAATTCCAGAAAAGAGAC | 327 |
| | | GCTTTCGAGCGTCTTTTTTCGTTTTGGTC | |
| | | C | |
| L3S2P55 | synthetic | CTCGGTACCAAAGACGAACAATAAGAC | 328 |
| | | GCTGAAAAGCGTCTTTTTTCGTTTTGGT | |
| | | CC | |
| L3S3P21 | synthetic | CCAATTATTGAAGGCCTCCCTAACGGG | 329 |
| | | GGGCCTTTTTTTGTTTCTGGTCTCCC | |
| L3S3P56 | synthetic | TTTTCGAAAAAACACCCTAACGGGTGT | 330 |
| | | TTTTTTGTTTCTGGTCTCCC | |
| L3S2P52 | synthetic | CTCGGTACCAAATCTAACTAAAAAGAC | 331 |
| | | GCTGAAAAGCGTCTTTTTTCGTTTTGGT | |
| | | CC | |
| L3S1P51 | synthetic | AAAAAAAAAAAAGGCCTCCCAAATCGG | 332 |
| | | GGGGCCTTTTTTATTGATAACAAAA | |
| L3S2P13 | synthetic | CTCGGTACCAAATTCCAGAAAAGAGAC | 333 |
| | | GCTTAACAGCGTCTTTTTTCGTTTTGGT | |
| | | CC | |
| L3S3P22 | synthetic | CCAATTATTGAAGGCCGCTAACGCGGC | 334 |
| | | CTTTTTTTGTTTCTGGTCTCCC | |
| L3S1P53 | synthetic | CCAATTATTGAAGGCCTCCCAAATCGG | 335 |
| | | GGGGCCTTTTTTATTGATAACAAAA | |
| L3S3P00 | synthetic | CCAATTATTGAAGGGGAGCGGGAAACC | 336 |
| | | GCTCCCCTTTTTTTGTTTCTGGTCTCCC | |
| L3S2P53 | synthetic | CTCGGTACCAAACCAATTATTGAAGAC | 337 |
| | | GCTGAAAAGCGTCTTTTTTCGTTTTGGT | |
| | | CC | |
| L3S2P15 | synthetic | CTCGGTACCAAATTCCAGAAAAGAGAC | 338 |
| | | GCTTTTAGAGCGTCTTTTTTCGTTTTGGT | |
| | | CC | |
| L3S2P31 | synthetic | CTCGGTACCAAATTCCAGAAAAGAGAC | 339 |
| | | GCTGAAAAGCGTCTTTTTTTTTTTTGGT | |
| | | CC | |
| L3S1P13 | synthetic | GACGAACAATAAGGCCTCCCTAACGGG | 340 |
| | | GGGCCTTTTTTATTGATAACAAAA | |
| L3S3P11 | synthetic | CCAATTATTGAACACCCTTCGGGGTGTT | 341 |
| | | TTTTTGTTTCTGGTCTCCC | |
| L3S3P23 | synthetic | CCAATTATTGAAGACGCTTAACAGCGT | 342 |
| | | CTTTTTTTGTTTCTGGTCTCCC | |
| L3S1P12 | synthetic | GACGAACAATAAGGCCTCCCGAAAGGG | 343 |
| | | GGGCCTTTTTTATTGATAACAAAA | |
| L3S3P52 | synthetic | TCTAACTAAAAACACCCTAACGGGTGT | 344 |
| | | TTTTTTGTTTCTGGTCTCCC | |
| L3S3P12 | synthetic | CCAATTATTGAACACCCGAAAGGGTGT | 345 |
| | | TTTTTTGTTTCTGGTCTCCC | |
| L3S1P41 | synthetic | AAAAAAAAAAAAGGCCTCCCAAATCGG | 346 |
| | | GGGGCCTTTTTTTTTTTTAACAAAA | |
| L3S2P24 | synthetic | CTCGGTACCAAATTCCAGAAAAGACAC | 347 |
| | | CCGAAAGGGTGTTTTTTCGTTTTGGTCC | |
| L3S2P14 | synthetic | CTCGGTACCAAATTCCAGAAAAGAGAC | 348 |
| | | GCTAAATCAGCGTCTTTTTTCGTTTTGG | |
| | | TCC | |
| L3S1P54 | synthetic | TTCCAGAAAAGAGGCCTCCCAAATCGG | 349 |
| | | GGGGCCTTTTTTATTGATAACAAAA | |
| L3S1P22 | synthetic | GACGAACAATAAGGCCGCAAATCGCGG | 350 |
| | | CCTTTTTTATTGATAACAAAA | |
| L3S2P00 | synthetic | CTCGGTACCAAATTCCAGAAAAGAGGG | 351 |
| | | GAGCGGGAAACCGCTCCCCTTTTTTCGT | |
| | | TTTGGTCC | |
| L3S1P47 | synthetic | TTTTCGAAAAAAGGCCTCCCAAATCGG | 352 |
| | | GGGGCCTTTTTTTATAGCAACAAAA | |
| L3S3P45 | synthetic | TTCCAGAAAAGACACCCTAACGGGTGT | 353 |
| | | TTTTTCGTTTTTGGTCTCCC | |
| L3S2P44 | synthetic | CTCGGTACCAAACCAATTATTGAAGAC | 354 |
| | | GCTGAAAAGCGTCTTTTTTTGTTTCGGT | |
| | | CC | |
| L3S3P54 | synthetic | TTCCAGAAAAGACACCCTAACGGGTGT | 355 |
| | | TTTTTTGTTTCTGGTCTCCC | |
| L3S3P31 | synthetic | CCAATTATTGAACACCCTAACGGGTGTT | 356 |
| | | TTTTTTTTTTTGGTCTCCC | |
| L3S1P11 | synthetic | GACGAACAATAAGGCCTCCCTTCGGGG | 357 |
| | | GGGCCTTTTTTATTGATAACAAAA | |
| L3S3P43 | synthetic | TCTAACTAAAAACACCCTAACGGGTGT | 358 |
| | | TTTTTCTTTTCTGGTCTCCC | |
| L3S3P42 | synthetic | GAAAAATAAAAACACCCTAACGGGTGT | 359 |
| | | TTTTATTTTTCTGGTCTCCC | |
| L3S2P42 | synthetic | CTCGGTACCAAAGAAAAATAAAAAGAC | 360 |
| | | GCTGAAAAGCGTCTTTTTATTTTTCGGT | |
| | | CC | |
| L3S3P14 | synthetic | CCAATTATTGAACACCCAAATCGGGTG | 361 |
| | | TTTTTTTGTTTCTGGTCTCCC | |
| L3S2P43 | synthetic | CTCGGTACCAAATCTAACTAAAAAGAC | 362 |
| | | GCTGAAAAGCGTCTTTTTTCTTTTCGGT | |
| | | CC | |
| L3S3P35 | synthetic | CCAATTATTGAACACCCTAACGGGTGTT | 363 |
| | | TTTTCGTTTTTGGTCTCCC | |
| L3S1P00 | synthetic | GACGAACAATAAGGGGAGCGGGAAAC | 364 |
| | | CGCTCCCCTTTTTTATTGATAACAAAA | |
| L3S3P47 | synthetic | TTTTCGAAAAAACACCCTAACGGGTGT | 365 |
| | | TTTTTTATAGCTGGTCTCCC | |
| L3S1P42 | synthetic | GAAAAATAAAAAGGCCTCCCAAATCGG | 366 |
| | | GGGGCCTTTTTATTTTTCAACAAAA | |
| L3S3P15 | synthetic | CCAATTATTGAACACCCTTTAGGGGTGT | 367 |
| | | TTTTTTGTTTCTGGTCTCCC | |
| L3S2P47 | synthetic | CTCGGTACCAAATTTTCGAAAAAAGAC | 368 |
| | | GCTGAAAAGCGTCTTTTTTTATAGCGGT | |
| | | CC | |
| L3S1P34 | synthetic | GACGAACAATAAGGCCTCCCAAATCGG | 369 |
| | | GGGGCCTTTTTTTGTTTCAACAAAA | |
| L3S2P34 | synthetic | CTCGGTACCAAATTCCAGAAAAGAGAC | 370 |
| | | GCTGAAAAGCGTCTTTTTTTGTTTCGGT | |
| | | CC | |
| L3S3P33 | synthetic | CCAATTATTGAACACCCTAACGGGTGTT | 371 |
| | | TTTTCTTTTCTGGTCTCCC | |
| L3S3P25 | synthetic | CCAATTATTGAAGCGGCTAACGCCGCT | 372 |
| | | TTTTTTGTTTCTGGTCTCCC | |
| L3S2P33 | synthetic | CTCGGTACCAAATTCCAGAAAAGAGAC | 373 |
| | | GCTGAAAAGCGTCTTTTTTCTTTTCGGT | |
| | | CC | |
| L3S3P55 | synthetic | GACGAACAATAACACCCTAACGGGTGT | 374 |
| | | TTTTTTGTTTCTGGTCTCCC | |
| L3S3P32 | synthetic | CCAATTATTGAACACCCTAACGGGTGTT | 375 |
| | | TTTATTTTTCTGGTCTCCC | |
| L3S1P44 | synthetic | CCAATTATTGAAGGCCTCCCAAATCGG | 376 |
| | | GGGGCCTTTTTTTGTTTCAACAAAA | |
| L3S1P43 | synthetic | TCTAACTAAAAAGGCCTCCCAAATCGG | 377 |
| | | GGGGCCTTTTTTCTTTTCAACAAAA | |
| L3S3P37 | synthetic | CCAATTATTGAACACCCTAACGGGTGTT | 378 |
| | | TTTTTATAGCTGGTCTCCC | |
| L3S2P37 | synthetic | CTCGGTACCAAATTCCAGAAAAGAGAC | 379 |
| | | GCTGAAAAGCGTCTTTTTTTATAGCGGT | |
| | | CC | |
| L3S1P32 | synthetic | GACGAACAATAAGGCCTCCCAAATCGG | 380 |
| | | GGGGCCTTTTTATTTTTCAACAAAA | |
| L3S2P46 | synthetic | CTCGGTACCAAAGACGAACAATAAGAC | 381 |
| | | GCTGAAAAGCGTCTTTTTTATTGATGGT | |
| | | CC | |
| L3S1P31 | synthetic | GACGAACAATAAGGCCTCCCAAATCGG | 382 |
| | | GGGGCCTTTTTTTTTTTTAACAAAA | |
| L3S1P33 | synthetic | GACGAACAATAAGGCCTCCCAAATCGG | 383 |
| | | GGGGCCTTTTTTCTTTTCAACAAAA | |
| L3S1P45 | synthetic | TTCCAGAAAAGAGGCCTCCCAAATCGG | 384 |
| | | GGGGCCTTTTTTCGTTTTAACAAAA | |
| L3S2P36 | synthetic | CTCGGTACCAAATTCCAGAAAAGAGAC | 385 |
| | | GCTGAAAAGCGTCTTTTTTATTGATGGT | |
| | | CC | |
| L2U1H10 | synthetic | GGGCGGTCAGATGATCGCCCTTTTTTTT | 386 |
| | | T | |
| L2U1H09 | synthetic | ACGGCCCTAGATAGGGCCGTTTTTTTTT | 387 |
| | | T | |
| L3S1P15 | synthetic | GACGAACAATAAGGCCTCCCTTTAGGG | 388 |
| | | GGGGCCTTTTTTATTGATAACAAAA | |
| L3S1P35 | synthetic | GACGAACAATAAGGCCTCCCAAATCGG | 389 |
| | | GGGGCCTTTTTTCGTTTTAACAAAA | |
| L1U1H11 | synthetic | CCCGCTTCGGCGGGTTTTTTTTT | 390 |
| L1U1H03 | synthetic | GATCCAGCCCATTCGTGGGCTGGATCTT | 391 |
| | | TTTTTTT | |
| L3S1P23 | synthetic | GACGAACAATAAGACGCTAAATCAGCG | 392 |
| | | TCTTTTTTATTGATAACAAAA | |
| L3S1P25 | synthetic | GACGAACAATAAGCGGCAAATCGCCGC | 393 |
| | | TTTTTTATTGATAACAAAA | |
| L3S2P32 | synthetic | CTCGGTACCAAATTCCAGAAAAGAGAC | 394 |
| | | GCTGAAAAGCGTCTTTTTATTTTTCGGT | |
| | | CC | |
| L3S3P46 | synthetic | GACGAACAATAACACCCTAACGGGTGT | 395 |
| | | TTTTTATTGATTGGTCTCCC | |
| L2U1H07 | synthetic | AGGCCTCCCAGATGGGGGGCCTTTTTTT | 396 |
| | | TTT | |
| L1U4H02 | synthetic | CCCGCATGCCATTCGTGGCATGCGGGTT | 397 |
| | | TTCTCTG | |
| L1U4H07 | synthetic | GATCCAGCTTCGGCTGGATCTTTTCTCT | 398 |
| | | G | |
| L1U4H11 | synthetic | CCCGCTTCGGCGGGTTTTCTCTG | 399 |
| L1U4H08 | synthetic | CCCGCATGTTCGCATGCGGGTTTTCTCT | 400 |
| | | G | |
| L1U4H01 | synthetic | TCGGTTACCGCTTCGGCGGTAACCGATT | 401 |
| | | TTCTCTG | |
| L1U4H06 | synthetic | TCGGTTACATGTTCGCATGTAACCGATT | 402 |
| | | TTCTCTG | |
| L2U1H08 | synthetic | TGCCGGGAAGATTCCCGGCATTTTTTTT | 403 |
| | | T | |
| L2U1H12 | synthetic | CGCCCGCAGATGCGGGCGTTTTTTTTT | 404 |
| L1U4H03 | synthetic | GATCCAGCCCATTCGTGGGCTGGATCTT | 405 |
| | | TTCTCTG | |
| L1U4H04 | synthetic | CGACGATGCCATTCGTGGCATCGTCGTT | 406 |
| | | TTCTCTG | |
| L1U4H09 | synthetic | CGACGATGTTCGCATCGTCGTTTTCTCT | 407 |
| | | G | |
| L3S1P24 | synthetic | GACGAACAATAACACCCAAATCGGGTG | 408 |
| | | TTTTTTATTGATAACAAAA | |
| L1U4H05 | synthetic | CGACGATACCATTCGTGGTATCGTCGTT | 409 |
| | | TTCTCTG | |
| L2U6H06 | synthetic | CTAAAGCGCCTAACCACGGCGCTTTAG | 410 |
| | | TTTTTTGTA | |
| L2U1H06 | synthetic | CTAAAGCGCCAGATGGCGCTTTAGTTTT | 41 |
| | | TTTTT | |
| L2U2H09 | synthetic | ACGGCCCTCGCAAGGGCCGTTTTTTTGT | 412 |
| | | A | |
| L2U6H09 | synthetic | ACGGCCCTTAACCACAGGGCCGTTTTTT | 413 |
| | | TGTA | |
| L2U2H06 | synthetic | CTAAAGCGCCCGCAGGCGCTTTAGTTTT | 414 |
| | | TTGTA | |
| L1U1H07 | synthetic | GATCCAGCTTCGGCTGGATCTTTTTTTT | 415 |
| | | T | |
| L2U6H07 | synthetic | AGGCCTCCCTAACCACGGGGGGCCTTT | 416 |
| | | TTTTGTA | |
| L1U4H12 | synthetic | CGACGTTCGCGTCGTTTTCTCTG | 417 |
| L1U1H08 | synthetic | CCCGCATGTTCGCATGCGGGTTTTTTTT | 418 |
| | | T | |
| L2U7H07 | synthetic | AGGCCTCCCGAACAGGGGGGCCTTTTT | 419 |
| | | GCAAC | |
| L1U4H10 | synthetic | CGACGATATTCGTATCGTCGTTTTCTCT | 420 |
| | | G | |
| L2U1H11 | synthetic | TAGCGTGCAGATGCACGCTATTTTTTTT | 421 |
| | | T | |
| L1U2H03 | synthetic | GATCCAGCCCATTCGTGGGCTGGATCTT | 422 |
| | | TTTTGGG | |
| L1U5H03 | synthetic | GATCCAGCCCATTCGTGGGCTGGATCTT | 423 |
| | | TATTGTC | |
| L2U4H09 | synthetic | ACGGCCCTCGCAAGGGCCGTTTTTGCA | 424 |
| | | AC | |
| L2U1H04 | synthetic | GCCGGAGCGGAGATCTGCTCCGGCTTT | 425 |
| | | TTTTTT | |
| L1U3H07 | synthetic | GATCCAGCTTCGGCTGGATCTTTTGTTG | 426 |
| | | G | |
| L2U6H12 | synthetic | CGCCCGCTAACCACGCGGGCGTTTTTTG | 427 |
| | | TA | |
| L2U7H06 | synthetic | CTAAAGCGCCGAACAGGCGCTTTAGTT | 428 |
| | | TTGCAAC | |
| L1U5H02 | synthetic | CCCGCATGCCATTCGTGGCATGCGGGTT | 429 |
| | | TATTGTC | |
| L1U2H01 | synthetic | TCGGTTACCGCTTCGGCGGTAACCGATT | 430 |
| | | TTTTGGG | |
| L1U5H07 | synthetic | GATCCAGCTTCGGCTGGATCTTTATTGT | 431 |
| | | C | |
| L1U2H04 | synthetic | CGACGATGCCATTCGTGGCATCGTCGTT | 432 |
| | | TTTTGGG | |
| L2U7H09 | synthetic | ACGGCCCTGAACAAGGGCCGTTTTTGC | 433 |
| | | AAC | |
| L1U5H09 | synthetic | CGACGATGTTCGCATCGTCGTTTATTGT | 434 |
| | | C | |
| L2U2H12 | synthetic | CGCCCGCCGCAGCGGGCGTTTTTTGTA | 435 |
| L1U1H09 | synthetic | CGACGATGTTCGCATCGTCGTTTTTTTT | 436 |
| | | T | |
| L1U5H01 | synthetic | TCGGTTACCGCTTCGGCGGTAACCGATT | 437 |
| | | TATTGTC | |
| L2U2H11 | synthetic | TAGCGTGCCGCAGCACGCTATTTTTTGT | 438 |
| | | A | |
| L2U4H12 | synthetic | CGCCCGCCGCAGCGGGCGTTTTGCAAC | 439 |
| L1U2H07 | synthetic | GATCCAGCTTCGGCTGGATCTTTTTTGG | 440 |
| | | G | |
| L2U4H10 | synthetic | GGGCGGTCCGCAGATCGCCCTTTTGCA | 441 |
| | | AC | |
| L2U3H09 | synthetic | ACGGCCCTCGCAAGGGCCGTTTTGTTG | 442 |
| | | AG | |
| L1U5H08 | synthetic | CCCGCATGTTCGCATGCGGGTTTATTGT | 443 |
| | | C | |
| L1U2H11 | synthetic | CCCGCTTCGGCGGGTTTTTTGGG | 444 |
| L1U5H04 | synthetic | CGACGATGCCATTCGTGGCATCGTCGTT | 445 |
| | | TATTGTC | |
| L1U2H02 | synthetic | CCCGCATGCCATTCGTGGCATGCGGGTT | 446 |
| | | TTTTGGG | |
| L1U5H06 | synthetic | TCGGTTACATGTTCGCATGTAACCGATT | 447 |
| | | TATTGTC | |
| L1U1H06 | synthetic | TCGGTTACATGTTCGCATGTAACCGATT | 448 |
| | | TTTTTTT | |
| L1U5H05 | synthetic | CGACGATACCATTCGTGGTATCGTCGTT | 449 |
| | | TATTGTC | |
| L1U8H12 | synthetic | CGACGTTCGCGTCGTTAAAATAA | 450 |
| L2U6H04 | synthetic | GCCGGAGCGGTAACCACCTGCTCCGGC | 451 |
| | | TTTTTTGTA | |
| L2U6H11 | synthetic | TAGCGTGCTAACCACGCACGCTATTTTT | 452 |
| | | TGTA | |
| L2U2H10 | synthetic | GGGCGGTCCGCAGATCGCCCTTTTTTGT | 452 |
| | | A | |
| L2U6H10 | synthetic | GGGCGGTCTAACCACGATCGCCCTTTTT | 453 |
| | | TGTA | |
| L2U2H07 | synthetic | AGGCCTCCCCGCAGGGGGGCCTTTTTTT | 454 |
| | | GTA | |
| L2U4H06 | synthetic | CTAAAGCGCCCGCAGGCGCTTTAGTTTT | 455 |
| | | GCAAC | |
| L2U1H05 | synthetic | GCCGGATCGGAGATCTGATCCGGCTTTT | 456 |
| | | TTTTT | |
| L1U1H01 | synthetic | TCGGTTACCGCTTCGGCGGTAACCGATT | 457 |
| | | TTTTTTT | |
| L1U5H10 | synthetic | CGACGATATTCGTATCGTCGTTTATTGT | 458 |
| | | C | |
| L1U2H08 | synthetic | CCCGCATGTTCGCATGCGGGTTTTTTGG | 459 |
| | | G | |
| L1U2H05 | synthetic | CGACGATACCATTCGTGGTATCGTCGTT | 460 |
| | | TTTTGGG | |
| L2U2H08 | synthetic | TGCCGGGACGCATCCCGGCATTTTTTGT | 461 |
| | | A | |
| L2U3H07 | synthetic | AGGCCTCCCCGCAGGGGGGCCTTTTGTT | 462 |
| | | GAG | |
| L2U6H08 | synthetic | TGCCGGGATAACCACTCCCGGCATTTTT | 463 |
| | | TGTA | |
| L2U6H05 | synthetic | GCCGGATCGGTAACCACCTGATCCGGC | 464 |
| | | TTTTTTGTA | |
| L1U3H03 | synthetic | GATCCAGCCCATTCGTGGGCTGGATCTT | 465 |
| | | TTGTTGG | |
| L2U1H03 | synthetic | TAGCGTGACCGGAGATTCGGTCACGCT | 466 |
| | | ATTTTTTTTT | |
| L1U2H09 | synthetic | CGACGATGTTCGCATCGTCGTTTTTTGG | 467 |
| | | G | |
| L1U5H12 | synthetic | CGACGTTCGCGTCGTTTATTGTC | 468 |
| L1U1H04 | synthetic | CGACGATGCCATTCGTGGCATCGTCGTT | 469 |
| | | TTTTTTT | |
| L1U3H02 | synthetic | CCCGCATGCCATTCGTGGCATGCGGGTT | 470 |
| | | TTGTTGG | |
| L1U2H06 | synthetic | TCGGTTACATGTTCGCATGTAACCGATT | 471 |
| | | TTTTGGG | |
| L2U7H05 | synthetic | GCCGGATCGGGAACACTGATCCGGCTT | 472 |
| | | TTGCAAC | |
| L1U1H02 | synthetic | CCCGCATGCCATTCGTGGCATGCGGGTT | 473 |
| | | TTTTTTT | |
| L1U2H10 | synthetic | CGACGATATTCGTATCGTCGTTTTTTGG | 474 |
| | | G | |
| L2U7H12 | synthetic | CGCCCGCGAACAGCGGGCGTTTTGCAA | 475 |
| | | C | |
| L1U1H05 | synthetic | CGACGATACCATTCGTGGTATCGTCGTT | 476 |
| | | TTTTTTT | |
| L1U3H04 | synthetic | CGACGATGCCATTCGTGGCATCGTCGTT | 477 |
| | | TTGTTGG | |
| L1U3H05 | synthetic | CGACGATACCATTCGTGGTATCGTCGTT | 478 |
| | | TTGTTGG | |
| L2U3H12 | synthetic | CGCCCGCCGCAGCGGGCGTTTGTTGAG | 479 |
| L2U2H04 | synthetic | GCCGGAGCGGCGCACTGCTCCGGCTTT | 480 |
| | | TTTGTA | |
| L1U3H06 | synthetic | TCGGTTACATGTTCGCATGTAACCGATT | 481 |
| | | TTGTTGG | |
| L1U3H11 | synthetic | CCCGCTTCGGCGGGTTTTGTTGG | 482 |
| L1U3H08 | synthetic | CCCGCATGTTCGCATGCGGGTTTTGTTG | 483 |
| | | G | |
| L1UIH10 | synthetic | CGACGATATTCGTATCGTCGTTTTTTTT | 484 |
| | | T | |
| L2U4H07 | synthetic | AGGCCTCCCCGCAGGGGGGCCTTTTTG | 485 |
| | | CAAC | |
| L2U7H08 | synthetic | TGCCGGGAGAACATCCCGGCATTTTGC | 486 |
| | | AAC | |
| L2U4H11 | synthetic | TAGCGTGCCGCAGCACGCTATTTTGCA | 487 |
| | | AC | |
| L2U3H06 | synthetic | CTAAAGCGCCCGCAGGCGCTTTAGTTT | 488 |
| | | GTTGAG | |
| L2U7H10 | synthetic | GGGCGGTCGAACAGATCGCCCTTTTGC | 489 |
| | | AAC | |
| L2U8H07 | synthetic | AGGCCTCCCTAACCACGGGGGGCCTTT | 490 |
| | | GTTGTAT | |
| L1U2H12 | synthetic | CGACGTTCGCGTCGTTTTTTGGG | 491 |
| L2U2H02 | synthetic | CCTGGTAAGACGCCGCAGCGTCTTATC | 492 |
| | | AGGTTTTTTGTA | |
| L1U8H03 | synthetic | GATCCAGCCCATTCGTGGGCTGGATCTT | 493 |
| | | AAAATAA | |
| L2U4H04 | synthetic | GCCGGAGCGGCGCACTGCTCCGGCTTT | 494 |
| | | TGCAAC | |
| L2U3H11 | synthetic | TAGCGTGCCGCAGCACGCTATTTGTTGA | 495 |
| | | G | |
| L2U7H02 | synthetic | CCTGGTAAGACGCGAACAGCGTCTTAT | 496 |
| | | CAGGTTTTGCAAC | |
| L1U7H08 | synthetic | CCCGCATGTTCGCATGCGGGTTATCAA | 497 |
| | | AA | |
| L1U3H09 | synthetic | CGACGATGTTCGCATCGTCGTTTTGTTG | 498 |
| | | G | |
| L2U2H05 | synthetic | GCCGGATCGGCGCACTGATCCGGCTTTT | 499 |
| | | TTGTA | |
| L2U4H08 | synthetic | TGCCGGGACGCATCCCGGCATTTTGCA | 500 |
| | | AC | |
| L2U5H06 | synthetic | CTAAAGCGCCGAACAGGCGCTTTAGTT | 501 |
| | | GTTGTAT | |
| L2U6H03 | synthetic | TAGCGTGACCGGTAACCACTCGGTCAC | 502 |
| | | GCTATTTTTTGTA | |
| L1U3H12 | synthetic | CGACGTTCGCGTCGTTTTGTTGG | 503 |
| L2U8H10 | synthetic | GGGCGGTCTAACCACGATCGCCCTTGTT | 504 |
| | | GTAT | |
| L1U3H10 | synthetic | CGACGATATTCGTATCGTCGTTTTGTTG | 505 |
| | | G | |
| L2U2H01 | synthetic | CCTGGTAAGACGCCGCAGCGTCGCATC | 506 |
| | | AGGTTTTTTGTA | |
| L2U8H12 | synthetic | CGCCCGCTAACCACGCGGGCGTTGTTG | 507 |
| | | TAT | |
| L2U1H02 | synthetic | CCTGGTAAGACGCAGATGCGTCTTATC | 508 |
| | | AGGTTTTTTTTT | |
| L2U2H03 | synthetic | TAGCGTGACCGGCGCATCGGTCACGCT | 509 |
| | | ATTTTTTGTA | |
| L2U3H04 | synthetic | GCCGGAGCGGCGCACTGCTCCGGCTTT | 510 |
| | | GTTGAG | |
| L2U3H08 | synthetic | TGCCGGGACGCATCCCGGCATTTGTTG | 511 |
| | | AG | |
| L2U8H08 | synthetic | TGCCGGGATAACCACTCCCGGCATTGTT | 512 |
| | | GTAT | |
| L2U4H01 | synthetic | CCTGGTAAGACGCCGCAGCGTCGCATC | 513 |
| | | AGGTTTTGCAAC | |
| L2U3H03 | synthetic | TAGCGTGACCGGCGCATCGGTCACGCT | 514 |
| | | ATTTGTTGAG | |
| L2U7H11 | synthetic | TAGCGTGCGAACAGCACGCTATTTTGC | 515 |
| | | AAC | |
| L2U4H03 | synthetic | TAGCGTGACCGGCGCATCGGTCACGCT | 516 |
| | | ATTTTGCAAC | |
| L2U7H04 | synthetic | GCCGGAGCGGGAACACTGCTCCGGCTT | 517 |
| | | TTGCAAC | |
| L1U8H09 | synthetic | CGACGATGTTCGCATCGTCGTTAAAAT | 518 |
| | | AA | |
| L2U5H05 | synthetic | GCCGGATCGGGAACACTGATCCGGCTT | 519 |
| | | GTTGTAT | |
| L2U5H10 | synthetic | GGGCGGTCGAACAGATCGCCCTTGTTG | 520 |
| | | TAT | |
| L2U5H12 | synthetic | CGCCCGCGAACAGCGGGCGTTGTTGTA | 521 |
| | | T | |
| L1U8H04 | synthetic | CGACGATGCCATTCGTGGCATCGTCGTT | 522 |
| | | AAAATAA | |
| L1U6H08 | synthetic | CCCGCATGTTCGCATGCGGGTTTCAACA | 523 |
| | | A | |
| L1U7H01 | synthetic | TCGGTTACCGCTTCGGCGGTAACCGATT | 524 |
| | | ATCAAAA | |
| L2U4H02 | synthetic | CCTGGTAAGACGCCGCAGCGTCTTATC | 525 |
| | | AGGTTTTGCAAC | |
| L2U3H10 | synthetic | GGGCGGTCCGCAGATCGCCCTTTGTTG | 526 |
| | | AG | |
| L2U6H01 | synthetic | CCTGGTAAGACGCTAACCACGCGTCGC | 527 |
| | | ATCAGGTTTTTTGTA | |
| L1U7H04 | synthetic | CGACGATGCCATTCGTGGCATCGTCGTT | 528 |
| | | ATCAAAA | |
| L2U3H01 | synthetic | CCTGGTAAGACGCCGCAGCGTCGCATC | 529 |
| | | AGGTTTGTTGAG | |
| L2U8H02 | synthetic | CCTGGTAAGACGCTAACCACGCGTCTT | 530 |
| | | ATCAGGTTGTTGTAT | |
| L1U7H12 | synthetic | CGACGTTCGCGTCGTTATCAAAA | 531 |
| L1U8H11 | synthetic | CCCGCTTCGGCGGGTTAAAATAA | 532 |
| L2U7H03 | synthetic | TAGCGTGACCGGGAACATCGGTCACGC | 533 |
| | | TATTTTGCAAC | |
| L1U8H05 | synthetic | CGACGATACCATTCGTGGTATCGTCGTT | 534 |
| | | AAAATAA | |
| L2U5H02 | synthetic | CCTGGTAAGACGCGAACAGCGTCTTAT | 535 |
| | | CAGGTTGTTGTAT | |
| L1U6H02 | synthetic | CCCGCATGCCATTCGTGGCATGCGGGTT | 536 |
| | | TCAACAA | |
| L2U1H01 | synthetic | CCTGGTAAGACGCAGATGCGTCGCATC | 537 |
| | | AGGTTTTTTTTT | |
| L2U5H04 | synthetic | GCCGGAGCGGGAACACTGCTCCGGCTT | 538 |
| | | GTTGTAT | |
| L2U3H05 | synthetic | GCCGGATCGGCGCACTGATCCGGCTTT | 539 |
| | | GTTGAG | |
| L1U7H03 | synthetic | GATCCAGCCCATTCGTGGGCTGGATCTT | 540 |
| | | ATCAAAA | |
| L1U6H04 | synthetic | CGACGATGCCATTCGTGGCATCGTCGTT | 541 |
| | | TCAACAA | |
| L2U5H08 | synthetic | TGCCGGGAGAACATCCCGGCATTGTTG | 542 |
| | | TAT | |
| L2U4H05 | synthetic | GCCGGATCGGCGCACTGATCCGGCTTTT | 543 |
| | | GCAAC | |
| L1U6H01 | synthetic | TCGGTTACCGCTTCGGCGGTAACCGATT | 544 |
| | | TCAACAA | |
| L1U7H07 | synthetic | GATCCAGCTTCGGCTGGATCTTATCAAA | 545 |
| | | A | |
| L1U8H07 | synthetic | GATCCAGCTTCGGCTGGATCTTAAAAT | 546 |
| | | AA | |
| L1U6H07 | synthetic | GATCCAGCTTCGGCTGGATCTTTCAACA | 547 |
| | | A | |
| L1U7H06 | synthetic | TCGGTTACATGTTCGCATGTAACCGATT | 548 |
| | | ATCAAAA | |
| L1UIH12 | synthetic | CGACGTTCGCGTCGTTTTTTTTT | 549 |
| L2U3H02 | synthetic | CCTGGTAAGACGCCGCAGCGTCTTATC | 550 |
| | | AGGTTTGTTGAG | |
| L2U5H09 | synthetic | ACGGCCCTGAACAAGGGCCGTTTGTTG | 551 |
| | | TAT | |
| L1U8H08 | synthetic | CCCGCATGTTCGCATGCGGGTTAAAAT | 552 |
| | | AA | |
| L1U8H10 | synthetic | CGACGATATTCGTATCGTCGTTAAAATA | 553 |
| | | A | |
| L1U7H09 | synthetic | CGACGATGTTCGCATCGTCGTTATCAAA | 554 |
| | | A | |
| L1U6H12 | synthetic | CGACGTTCGCGTCGTTTCAACAA | 555 |
| L2U5H07 | synthetic | AGGCCTCCCGAACAGGGGGGCCTTTGT | 556 |
| | | TGTAT | |
| L1U7H05 | synthetic | CGACGATACCATTCGTGGTATCGTCGTT | 557 |
| | | ATCAAAA | |
| L1U8H02 | synthetic | CCCGCATGCCATTCGTGGCATGCGGGTT | 558 |
| | | AAAATAA | |
| L2U6H02 | synthetic | CCTGGTAAGACGCTAACCACGCGTCTT | 559 |
| | | ATCAGGTTTTTTGTA | |
| L1U8H06 | synthetic | TCGGTTACATGTTCGCATGTAACCGATT | 560 |
| | | AAAATAA | |
| L2U8H05 | synthetic | GCCGGATCGGTAACCACCTGATCCGGC | 561 |
| | | TTGTTGTAT | |
| L1U7H02 | synthetic | CCCGCATGCCATTCGTGGCATGCGGGTT | 562 |
| | | ATCAAAA | |
| L1U8H01 | synthetic | TCGGTTACCGCTTCGGCGGTAACCGATT | 563 |
| | | AAAATAA | |
| L1U7H11 | synthetic | CCCGCTTCGGCGGGTTATCAAAA | 564 |
| L1U6H11 | synthetic | CCCGCTTCGGCGGGTTTCAACAA | 565 |
| L1U6H06 | synthetic | TCGGTTACATGTTCGCATGTAACCGATT | 566 |
| | | TCAACAA | |
| L1U7H10 | synthetic | CGACGATATTCGTATCGTCGTTATCAAA | 567 |
| | | A | |
| L2U8H09 | synthetic | ACGGCCCTTAACCACAGGGCCGTTTGTT | 568 |
| | | GTAT | |
| L2U7H01 | synthetic | CCTGGTAAGACGCGAACAGCGTCGCAT | 569 |
| | | CAGGTTTTGCAAC | |
| L2U8H01 | synthetic | CCTGGTAAGACGCTAACCACGCGTCGC | 570 |
| | | ATCAGGTTGTTGTAT | |
| L1U6H09 | synthetic | CGACGATGTTCGCATCGTCGTTTCAACA | 571 |
| | | A | |
| L1U6H05 | synthetic | CGACGATACCATTCGTGGTATCGTCGTT | 572 |
| | | TCAACAA | |
| L1U6H03 | synthetic | GATCCAGCCCATTCGTGGGCTGGATCTT | 573 |
| | | TCAACAA | |
| L2U5H03 | synthetic | TAGCGTGACCGGGAACATCGGTCACGC | 574 |
| | | TATTGTTGTAT | |
| L2U8H04 | synthetic | GCCGGAGCGGTAACCACCTGCTCCGGC | 575 |
| | | TTGTTGTAT | |
| L2U5H01 | synthetic | CCTGGTAAGACGCGAACAGCGTCGCAT | 576 |
| | | CAGGTTGTTGTAT | |
| L2U8H03 | synthetic | TAGCGTGACCGGTAACCACTCGGTCAC | 577 |
| | | GCTATTGTTGTAT | |
| L1U6H10 | synthetic | CGACGATATTCGTATCGTCGTTTCAACA | 578 |
| | | A | |
| L2U8H11 | synthetic | TAGCGTGCTAACCACGCACGCTATTGTT | 579 |
| | | GTAT | |
| L2U8H06 | synthetic | CTAAAGCGCCTAACCACGGCGCTTTAG | 580 |
| | | TTGTTGTAT | |
| L2U5H11 | synthetic | TAGCGTGCGAACAGCACGCTATTGTTG | 581 |
| | | TAT | |
| BBa_J61048 | rnpB-T1 | ccggcttatcggtcagtttcacctgatttacgtaaaaacccgcttc | 820 |
| | | ggcgggtttttgcttttggaggggcagaaagatgaatgactgtcc | |
| | | acgacgctatacccaaaagaaa | |

APPENDIX 2: Promotor Sequences

| | | | SEQ |
|---|---|---|---|
| | | | ID |
| Name | Description | Promoter Sequence | NO: |
| BBa_J23100 | constitutive promoter | . . . ggctagctcagtcctaggtacagtgctagc | 582 |
| | family member | | |
| BBa_J23101 | constitutive promoter | . . . agctagctcagtcctaggtattatgctagc | 583 |
| | family member | | |
| BBa_J23102 | constitutive promoter | . . . agctagctcagtcctaggtactgtgctagc | 584 |
| | family member | | |
| BBa_J23103 | constitutive promoter | . . . agctagctcagtcctagggattatgctagc | 585 |
| | family member | | |
| BBa_J23104 | constitutive promoter | . . . agctagctcagtcctaggtattgtgctagc | 586 |
| | family member | | |
| BBa_J23105 | constitutive promoter | . . . ggctagctcagtcctaggtactatgctagc | 587 |
| | family member | | |
| BBa_J23106 | constitutive promoter | . . . ggctagctcagtcctaggtatagtgctagc | 588 |
| | family member | | |
| BBa_J23108 | constitutive promoter | . . . agctagctcagtcctaggtataatgctagc | 589 |
| | family member | | |
| BBa_J23109 | constitutive promoter | . . . agctagctcagtcctagggactgtgctagc | 590 |
| | family member | | |
| BBa_J23110 | constitutive promoter | . . . ggctagctcagtcctaggtacaatgctagc | 592 |
| | family member | | |
| BBa_J23111 | constitutive promoter | . . . ggctagctcagtcctaggtatagtgctagc | 593 |
| | family member | | |
| BBa_J23112 | constitutive promoter | . . . agctagctcagtcctagggattatgctagc | 594 |
| | family member | | |
| BBa_J23113 | constitutive promoter | . . . ggctagctcagtcctagggattatgctagc | 595 |
| | family member | | |
| BBa_J23114 | constitutive promoter | . . . ggctagctcagtcctaggtacaatgctagc | 596 |
| | family member | | |
| BBa_J23115 | constitutive promoter | . . . agctagctcagcccttggtacaatgctagc | 597 |
| | family member | | |
| BBa_J23116 | constitutive promoter | . . . agctagctcagtcctagggactatgctagc | 598 |
| | family member | | |
| BBa_J23117 | constitutive promoter | . . . agctagctcagtcctagggattgtgctagc | 599 |
| | family member | | |
| BBa_J23118 | constitutive promoter | . . . ggctagctcagtcctaggtattgtgctagc | 600 |
| | family member | | |
| BBa_J44002 | pBAD reverse | . . . aaagtgtgacgccgtgcaaataatcaatgt | 601 |
| BBa_K256002 | J23101:GFP | . . . caccttcgggtgggcctttctgcgtttata | 602 |
| BBa_K823004 | Anderson promoter | . . . ggctagctcagtcctaggtacagtgctagc | 603 |
| | J23100 | | |
| BBa_K823005 | Anderson promoter | . . . agctagctcagtcctaggtattatgctagc | 604 |
| | J23101 | | |
| BBa_K823006 | Anderson promoter | . . . agctagctcagtcctaggtactgtgctagc | 605 |
| | J23102 | | |
| BBa_K823007 | Anderson promoter | . . . agctagctcagtcctagggattatgctagc | 606 |
| | J23103 | | |
| BBa_K823008 | Anderson promoter | . . . ggctagctcagtcctaggtatagtgctagc | 607 |
| | J23106 | | |
| BBa_K823010 | Anderson promoter | . . . ggctagctcagtcctagggattatgctagc | 608 |
| | J23113 | | |
| BBa_K823011 | Anderson promoter | . . . ggctagctcagtcctaggtacaatgctagc | 609 |
| | J23114 | | |
| BBa_K823013 | Anderson promoter | . . . agctagctcagtcctagggattgtgctagc | 610 |
| | J23117 | | |
| BBa_K823014 | Anderson promoter | . . . ggctagctcagtcctaggtattgtgctagc | 611 |
| | J23118 | | |
| BBa_I14018 | P(Bla) | . . . gtttatacataggcgagtactctgttatgg | 612 |
| BBa_I14033 | P(Cat) | . . . agaggttccaactttcaccataatgaaaca | 613 |
| BBa_I14034 | P(Kat) | . . . taaacaactaacggacaattctacctaaca | 614 |
| BBa_J23119 | constitutive promoter | . . . agctagctcagtcctaggtataatgctagc | 615 |
| | family member | | |
| BBa_J23150 | 1 bp mutant from J23107 | . . . ggctagctcagtcctaggtattatgctagc | 616 |
| BBa_K1330002 | Constitutive promoter | . . . ggctagctcagtcctaggtactatgctagc | 617 |
| | (J23105) | | |
| BBa_I742126 | Reverse lambda cI- | . . . gaggtaaaatagtcaacacgcacggtgtta | 618 |
| | regulated promoter | | |
| BBa_J01006 | Key Promoter absorbs 3 | . . . caggccggaataactccctataatgcgcca | 619 |
| BBa_J23151 | 1 bp mutant from J23114 | . . . ggctagctcagtcctaggtacaatgctagc | 620 |
| BBa_J48104 | NikR promoter, a protein | . . . gacgaatacttaaaatcgtcatacttattt | 623 |
| | of the ribbon helix-helix | | |
| | family of trancription | | |
| | factors that repress expre | | |
| BBa_J54200 | lacq_Promoter | . . . aaacctttcgcggtatggcatgatagcgcc | 624 |
| BBa_J56015 | lacIQ - promoter sequence | . . . tgatagcgcccggaagagagtcaattcagg | 625 |
| BBa_J64951 | E. Coli CreABCD | . . . ttatttaccgtgacgaactaattgctcgtg | 626 |
| | phosphate sensing operon | | |
| | promoter | | |
| BBa_K119000 | Constitutive weak | . . . ttatgcttccggctcgtatgttgtgtggac | 627 |
| | promoter of lacZ | | |
| BBa_K119001 | Mutated LacZ promoter | . . . ttatgcttccggctcgtatggtgtgtggac | 628 |
| BBa_K137085 | optimized (TA) repeat | . . . tgacaatatatatatatatataatgctagc | 629 |
| | constitutive promoter with | | |
| | 13 bp between −10 and - | | |
| | 35 elements | | |
| BBa_K137086 | optimized (TA) repeat | . . . acaatatatatatatatatataatgctagc | 630 |
| | constitutive promoter with | | |
| | 15 bp between -10 and | | |
| | −35 elements | | |
| BBa_K137087 | optimized (TA) repeat | . . . aatatatatatatatatatataatgctagc | 631 |
| | constitutive promoter with | | |
| | 17 bp between -10 and | | |
| | -35 elements | | |
| BBa_K137088 | optimized (TA) repeat | . . . tatatatatatatatatatataatgctagc | 632 |
| | constitutive promoter with | | |
| | 19 bp between -10 and | | |
| | -35 elements | | |
| BBa_K137089 | optimized (TA) repeat | . . . tatatatatatatatatatataatgctagc | 633 |
| | constitutive promoter with | | |
| | 21 bp between -10 and | | |
| | -35 elements | | |
| BBa_K137090 | optimized (A) repeat | . . . aaaaaaaaaaaaaaaaaatataatgctagc | 634 |
| | constitutive promoter with | | |
| | 17 bp between -10 and | | |
| | -35 elements | | |
| BBa_K137091 | optimized (A) repeat | . . . aaaaaaaaaaaaaaaaaatataatgctagc | 635 |
| | constitutive promoter with | | |
| | 18 bp between -10 and | | |
| | -35 elements | | |
| BBa_K1585100 | Anderson Promoter with | . . . ggaattgtgagcggataacaatttcacaca | 636 |
| | lacI binding site | | |
| BBa_K1585101 | Anderson Promoter with | . . . ggaattgtgagcggataacaatttcacaca | 637 |
| | lacI binding site | | |
| BBa_K1585102 | Anderson Promoter with | . . . ggaattgtgagcggataacaatttcacaca | 638 |
| | lacI binding site | | |
| BBa_K1585103 | Anderson Promoter with | . . . ggaattgtgagcggataacaatttcacaca | 639 |
| | lacI binding site | | |
| BBa_K1585104 | Anderson Promoter with | . . . ggaattgtgagcggataacaatttcacaca | 640 |
| | lacI binding site | | |
| BBa_K1585105 | Anderson Promoter with | . . . ggaattgtgagcggataacaatttcacaca | 641 |
| | lacI binding site | | |
| BBa_K1585106 | Anderson Promoter with | . . . ggaattgtgagcggataacaatttcacaca | 642 |
| | lacI binding site | | |
| BBa_K1585110 | Anderson Promoter with | . . . ggaattgtgagcggataacaatttcacaca | 643 |
| | lacI binding site | | |
| BBa_K1585113 | Anderson Promoter with | . . . ggaattgtgagcggataacaatttcacaca | 644 |
| | lacI binding site | | |
| BBa_K1585115 | Anderson Promoter with | . . . ggaattgtgagcggataacaatttcacaca | 645 |
| | lacI binding site | | |
| BBa_K1585116 | Anderson Promoter with | . . . ggaattgtgagcggataacaatttcacaca | 646 |
| | lacI binding site | | |
| BBa_K1585117 | Anderson Promoter with | . . . ggaattgtgagcggataacaatttcacaca | 647 |
| | lacI binding site | | |
| BBa_K1585118 | Anderson Promoter with | . . . ggaattgtgagcggataacaatttcacaca | 648 |
| | lacI binding site | | |
| BBa_K1585119 | Anderson Promoter with | . . . ggaattgtgagcggataacaatttcacaca | 649 |
| | lacI binding site | | |
| BBa_K2486171 | A reverse complement | . . . cattgtacctaggactgagctagccataaa | 650 |
| | version of BBa_J23114 | | |
| BBa_K418000 | IPTG inducible Lac | . . . ttgtgagcggataacaagatactgagcaca | 651 |
| | promoter cassette | | |
| BBa_M13101 | M13K07 gene I promoter | . . . cctgtttttatgttattctctctgtaaagg | 652 |
| BBa_M13102 | M13K07 gene II promoter | . . . aaatatttgcttatacaatcttcctgtttt | 653 |
| BBa_M13103 | M13K07 gene III | . . . gctgataaaccgatacaattaaaggctcct | 654 |
| | promoter | | |
| BBa_M13104 | M13K07 gene IV | . . . ctcttctcagcgtcttaatctaagctatcg | 655 |
| | promoter | | |
| BBa_M13105 | M13K07 gene V promoter | . . . atgagccagttcttaaaatcgcataaggta | 656 |
| BBa_M13106 | M13K07 gene VI | . . . ctattgattgtgacaaaataaacttattcc | 657 |
| | promoter | | |
| BBa_M13108 | M13K07 gene VIII | . . . gtttcgcgcttggtataatcgctgggggtc | 658 |
| | promoter | | |
| BBa_M13110 | M13110 | . . . cttgcttctgactataatagtcagggtaa | 659 |
| BBa_M31519 | Modified promoter | . . . aaaccgatacaattaaaggctcctgctagc | 660 |
| | sequence of g3. | | |
| BBa_R1074 | Constitutive Promoter I | . . . caccacactgatagtgctagtgtagatcac | 661 |
| BBa_R1075 | Constitutive Promoter II | . . . gccggaataactccctataatgcgccacca | 662 |
| BBa_S03331 | -- Specify Parts List -- | ttgacaagcttttcctcagctccgtaaact | 663 |
| BBa_J23107 | constitutive promoter | . . . ggctagctcagccctaggtattatgctagc | 664 |
| | family member | | |
| BBa_K088007 | GlnRS promoter | . . . catacgccgttatacgttgtttacgctttg | 665 |
| BBa_K137029 | constitutive promoter with | . . . atatatatatatatataatggaagcgtttt | 666 |
| | (TA)10 between -10 and | | |
| | -35 elements | | |
| BBa_K137030 | constitutive promoter with | . . . atatatatatatatataatggaagcgtttt | 667 |
| | (TA)9 between -10 and | | |
| | -35 elements | | |
| BBa_K137031 | constitutive promoter with | . . . ccccgaaagcttaagaatataattgtaagc | 668 |
| | (C)10 between -10 and | | |
| | -35 elements | | |
| BBa_K137032 | constitutive promoter with | . . . ccccgaaagcttaagaatataattgtaagc | 669 |
| | (C)12 between -10 and | | |
| | -35 elements | | |
| BBa_K1824896 | J23100 + RBS | . . . gattaaagaggagaaatactagagtactag | 670 |
| BBa_K256018 | J23119:IFP | . . . caccttcgggtgggcctttctgcgtttata | 671 |
| BBa_K256020 | J23119:HO1 | . . . caccttcggggggcctttctgcgtttata | 672 |
| BBa_K256033 | Infrared signal reporter | . . . caccttcggggggcctttctgcgtttata | 673 |
| | (J23119:IFP:J23119:HO1) | | |
| BBa_K292000 | Double terminator + | . . . ggctagctcagtcctaggtacagtgctagc | 674 |
| | constitutive promoter | | |
| BBa_K292001 | Double terminator + | . . . tgctagctactagagattaaagaggagaaa | 675 |
| | Constitutive promoter + | | |
| | Strong RBS | | |
| BBa_K418002 | IPTG inducible Lac | . . . ttgtgagcggataacaagatactgagcaca | 676 |
| | promoter cassette | | |
| BBa_K418003 | IPTG inducible Lac | . . . ttgtgagcggataacaagatactgagcaca | 677 |
| | promoter cassette | | |
| BBa_I0500 | Inducible pBad/araC | . . . gtttctccatacccgtttttttgggctagc | 678 |
| | promoter | | |
| BBa_I1051 | Lux cassette right | . . . tgttatagtcgaatacctctggcggtgata | 679 |
| | promoter | | |
| BBa_I12006 | Modified lamdba Prm | . . . attacaaactttcttgtatagatttaacgt | 680 |
| | promoter (repressed by | | |
| | 434 cI) | | |
| BBa_I12007 | Modified lambda Prm | . . . atttataaatagtggtgatagatttaacgt | 681 |
| | promoter (OR-3 | | |
| | obliterated) | | |
| BBa_I12036 | Modified lamdba Prm | . . . tttcttgtatagatttacaatgtatcttgt | 682 |
| | promoter (cooperative | | |
| | repression by 434 cI) | | |
| BBa_I12040 | Modified lambda P(RM) | . . . tttcttgtagatacttacaatgtatcttgt | 683 |
| | promoter: -10 region from | | |
| | P(L) and cooperatively | | |
| | repressed by 434 cI | | |
| BBa_I12210 | plac Or2-62 (positive) | . . . ctttatgcttccggctcgtatgttgtgtgg | 684 |
| BBa_I13406 | Pbad/AraC with extra | . . . ttttttgggctagcaagctttaccatggat | 685 |
| | REN sites | | |
| BBa_I13453 | Pbad promoter | . . . tgtttctccataccgtttttttgggctagc | 686 |
| BBa_I14015 | P(Las) TetO | . . . ttttggtacactccctatcagtgatagaga | 687 |
| BBa_I14016 | P(Las) CIO | . . . ctttttggtacactacctctggcggtgata | 688 |
| BBa_I14017 | P(Rhl) | . . . tacgcaagaaaatggtttgttatagtcgaa | 689 |
| BBa_I721001 | Lead Promoter | . . . gaaaaccttgtcaatgaagagcgatctatg | 690 |
| BBa_I723020 | Pu | . . . ctcaaagcgggccagccgtagccgttacgc | 691 |
| BBa_I731004 | FecA promoter | . . . ttctcgttcgactcatagctgaacacaaca | 692 |
| BBa_I739104 | Double Promoter | . . . gttctttaattatttaagtgttctttaatt | 693 |
| | (LuxR/HSL, positive/ | | |
| | P22 cII, negative) | | |
| BBa_I739105 | Double Promoter | . . . cgtgcgtgtgataacaccgtgcgtgttga | 694 |
| | (LuxR/HSL, positive/cI, | | |
| | negative) | | |
| BBa_I741018 | Right facing promoter (for | . . . gttacgtttatcgcggtgattgttacttat | 695 |
| | xylF) controlled by xylR | | |
| | and CRP-cAMP | | |
| BBa_I741019 | Right facing promoter (for | . . . gcaaaataaaatggaatgatgaaactgggt | 696 |
| | xylA) controlled by xylR | | |
| | and CRP-CAMP | | |
| BBa_I741020 | promoter to xylF without | . . . gttacgtttatcgcggtgattgttacttat | 697 |
| | CRP and several binding | | |
| | sites for xylR | | |
| BBa_I741021 | promoter to xylA without | . . . atttcacactgctattgagataattcacaa | 698 |
| | CRP and several binding | | |
| | sites for xylR | | |
| BBa_I746104 | P2 promoter in agr operon | . . . agattgtactaaatcgtataatgacagtga | 699 |
| | from S. aureus | | |
| BBa_I746360 | PF promoter from P2 | . . . gacatctccggcgcaactgaaaataccact | 700 |
| | phage | | |
| BBa_I746361 | PO promoter from P2 | . . . gaggatgcgcatcgtcgggaaactgatgcc | 701 |
| | phage | | |
| BBa_I746362 | PP promoter from P2 | . . . catccgggactgatggcggaggatgcgcat | 702 |
| | phage | | |
| BBa_I746363 | PV promoter from P2 | . . . aacttttatatattgtgcaatctcacatgc | 703 |
| | phage | | |
| BBa_I746364 | Psid promoter from P4 | . . . tgttgtccggtgtacgtcacaattttctta | 704 |
| | phage | | |
| BBa_I746365 | PLL promoter from P4 | . . . gtctgctgaaaatattcacaaaataaagcg | 705 |
| | phage | | |
| BBa_I751501 | plux-cI hybrid promoter | . . . gtgttgatgcttttatcaccgccagtggta | 706 |
| BBa_I751502 | plux-lac hybrid promoter | . . . agtgtgtggaattgtgagcggataacaatt | 707 |
| BBa_I760005 | Cu-sensitive promoter | atgacaaaattgtcat | 708 |
| BBa_I761011 | CinR, CinL and glucose | . . . acatcttaaaagttttagtatcatattcgt | 709 |
| | controlled promotor | | |
| BBa_I765001 | UV promoter | . . . ctgaaagcgcataccgctatggagggggtt | 710 |
| BBa_I765007 | Fe and UV promoters | . . . ctgaaagcgcataccgctatggagggggtt | 711 |
| BBa_J01005 | pspoIIE promoter (spo0A | . . . aacgaatataacaggtgggagatgagagga | 712 |
| | J01004, positive) | | |
| BBa_J03007 | Maltose specific promotor | . . . aatatttcctcattttccacagtgaagtga | 713 |
| BBa_J06403 | RhIR promoter repressible | . . . tacgcaagaaaatggtttgttatagtcgaa | 714 |
| | by CI | | |
| BBa_J07007 | ctx promoter | . . . atttaattgttttgatcaattatttttctg | 715 |
| BBa_J102001 | Reverse Lux Promoter | . . . tcttgcgtaaacctgtacgatcctacaggt | 716 |
| BBa_J13210 | pOmpR dependent POPS | . . . attattctgcatttttggggagaatggact | 717 |
| | producer | | |
| BBa_J15502 | copA promoter | . . . ccttgctggaaggtttaacctttatcacag | 718 |
| BBa_J16101 | BanAp - Banana-induced | atgatgtgtccatggatta | 719 |
| | Promoter | | |
| BBa_J16105 | HelPp - “Help” | atgatagacgatgtgcggacaacgtg | 720 |
| | Dependant promoter | | |
| BBa_J45503 | hybB Cold Shock | . . . cattagccgccaccatggggttaagtagca | 721 |
| | Promoter | | |
| BBa_J58100 | AND-type promoter | . . . atttataaatagtggtgatagatttaacgt | 722 |
| | synergistically activated | | |
| | by cl and CRP | | |
| BBa_J61051 | [Psal1] | . . . ataaagccatcacgagtaccatagaggatc | 723 |
| BBa_J61054 | [HIP-1] Promoter | . . . tttgtcttttcttgcttaataatgttgtca | 724 |
| BBa_J61055 | [HIP-1fnr] Promoter | . . . tttgtcttttcttgcttaataatgttgtca | 725 |
| BBa_J64000 | rhlI promoter | . . . atcctcctttagtcttccccctcatgtgtg | 726 |
| BBa_J64010 | lasI promoter | . . . taaaattatgaaattigcataaattctica | 727 |
| BBa_J64712 | LasR/LasI Inducible & | . . . gaaatctggcagtttttggtacacgaaagc | 728 |
| | RHLR/RHLI repressible | | |
| | Promoter | | |
| BBa_J64800 | RHLR/RHLI Inducible & | . . . tgccagttctggcaggtctaaaaagtgttc | 729 |
| | LasR/LasI repressible | | |
| | Promoter | | |
| BBa_J64804 | The promoter region | . . . cacagaacttgcatttatataaagggaaag | 730 |
| | (inclusive of regulator | | |
| | binding sites) of the B. | | |
| | subtilis RocDEF operon | | |
| BBa_K091107 | pLux/cI Hybrid Promoter | . . . acaccgtgcgtgttgatatagtcgaataaa | 731 |
| BBa_K091117 | pLas promoter | . . . aaaattatgaaatttgtataaattcttcag | 732 |
| BBa_K091143 | pLas/cI Hybrid Promoter | . . . ggttctttttggtacctctggcggtgataa | 733 |
| BBa_K091146 | pLas/Lux Hybrid | . . . tgtaggatcgtacaggtataaattcttcag | 734 |
| | Promoter | | |
| BBa_K091156 | pLux | . . . caagaaaatggtttgttatagtcgaataaa | 735 |
| BBa_K091157 | pLux/Las Hybrid | . . . ctatctcatttgctagtatagtcgaataaa | 736 |
| | Promoter | | |
| BBa_K100000 | Natural Xylose Regulated | . . . gttacgtttatcgcggtgattgttacttat | 737 |
| | Bi-Directional Operator | | |
| BBa_K100001 | Edited Xylose Regulated | . . . gttacgtttatcgcggtgattgttacttat | 738 |
| | Bi-Directional Operator 1 | | |
| BBa_K100002 | Edited Xylose Regulated | . . . gttacgtttatcgcggtgattgttacttat | 739 |
| | Bi-Directional Operator 2 | | |
| BBa_K112118 | rmB P1 promoter | . . . ataaatgcttgactctgtagcgggaaggcg | 740 |
| BBa_K112320 | {<ftsAZ promoter>} in | . . . aaaactggtagtaggactggagattggtac | 741 |
| | BBb format | | |
| BBa_K112322 | {Pdps} in BBb format | . . . gggacacaaacatcaagaggatatgagatt | 742 |
| BBa_K112402 | promoter for FabA gene - | . . . gtcaaaatgaccgaaacgggtggtaacttc | 743 |
| | Membrane Damage and | | |
| | Ultrasound Senstitive | | |
| BBa_K112405 | Promoter for CadA and | . . . agtaatcttatcgccagtttggtctggtca | 744 |
| | CadB genes | | |
| BBa_K112406 | cadC promoter | . . . agtaatcttatcgccagtttggtctggtca | 745 |
| BBa_K112701 | hns promoter | . . . aattctgaacaacatccgtactcttcgtgc | 746 |
| BBa_K112900 | Pbad | . . . tcgataagattaccgatcttacctgaagct | 747 |
| BBa_K116001 | nhaA promoter, that can | . . . cgatctattcacctgaaagagaaataaaaa | 748 |
| | be regulated by pH and | | |
| | nhaR protein. | | |
| BBa_K116401 | external phosphate | . . . attaatgatcgcaacctatttattacaaca | 749 |
| | sensing promoter | | |
| BBa_K116500 | OmpF promoter that is | . . . aaacgttagtttgaatggaaagatgcctgc | 750 |
| | activated or repressesed | | |
| | by OmpR according to | | |
| | osmolarity. | | |
| BBa_K116603 | pRE promoter from λ | . . . tttgcacgaaccatatgtaagtatttcctt | 751 |
| | phage | | |
| BBa_K117002 | LsrA promoter (indirectly | . . . taacacttatttaattaaaaagaggagaaa | 752 |
| | activated by AI-2) | | |
| BBa_K118011 | PostA (glucose- | . . . tagaaacaaaatgtaacatctctatggaca | 753 |
| | repressible promoter) | | |
| BBa_K121011 | promoter (lacI regulated) | . . . acaggaaacagctatgaccatgattacgcc | 754 |
| BBa_K135000 | pCpxR (CpxR responsive | . . . agcgacgtctgatgacgtaatttctgcctc | 755 |
| | promoter) | | |
| BBa_K136010 | fliA promoter | . . . gttcactctataccgctgaaggtgtaatgg | 756 |
| BBa_K145150 | Hybrid promoter: HSL- | . . . tagtttataatttaagtgttctttaatttc | 757 |
| | LuxR activated, P22 C2 | | |
| | repressed | | |
| BBa_K1520010 | Prlux-rbs-rfp-Ter | . . . caccttcggggggcctttctgcgtttata | 758 |
| BBa_K1520515 | MC7-rbs-luxI-Ter- | . . . caccttcggggggcctttctgcgtttata | 759 |
| | Pcons2-rbs-luxR-Ter- | | |
| | Prlux-rbs-OMPA-golB- | | |
| | rbs-luxI-Ter. | | |
| BBa_K1520516 | MC31-rbs-luxI-Ter- | . . . caccttcgggtgggcctttctgcgtttata | 760 |
| | Pcons2-rbs-luxR-Ter- | | |
| | Prlux-rbs-OMPA-golB- | | |
| | rbs-luxI-Ter | | |
| BBa_K1660005 | RFP controlled by the | . . . caccttcggggggcctttctgcgtttata | 761 |
| | PompR promoter | | |
| BBa_K180000 | Hybrid promoter (trp & | . . . cgagcacttcaccaacaaggaccatagcat | 762 |
| | lac regulated -- tac pR) | | |
| BBa_K180002 | tac pR testing plasmid | . . . caccttcggggggcctttctgcgtttata | 763 |
| | (GFP) | | |
| BBa_K180003 | PTAC testing plasmid | . . . catggcatggatgaactatacaaataataa | 764 |
| | (GFP) - basic | | |
| BBa_K180004 | Game of Life - Primary | . . . caccttcggggggcctttctgcgtttata | 765 |
| | plasmid | | |
| BBa_K180005 | GoL - Primary plasmid | . . . caccttcgggtgggcctttctgcgtttata | 766 |
| | (part 1)/RPS - Paper | | |
| | primary plasmid (part 1) | | |
| | [LuxR generator] | | |
| BBa_K180006 | Game of Life - Primary | . . . caccttcggggggcctttctgcgtttata | 767 |
| | plasmid (part 2) [lux pR, | | |
| | GFP and LacI generator] | | |
| BBa_K180007 | Game of Life - Secondary | . . . caccttcgggtgggcctttctgcgtttata | 768 |
| | plasmid [tac pR, LuxI | | |
| | generator | | |
| BBa_K180010 | Rock-paper-scissors - | . . . caccttcggggggcctttctgcgtttata | 769 |
| | Rock primary plasmid | | |
| BBa_K180011 | Rock - Primary plasmid | . . . caccttcggggggcctttctgcgtttata | 770 |
| | (part 1) [RhlR generator] | | |
| BBa_K180012 | Rock - Primary plasmid | . . . caccttcggggggcctttclgcgtttata | 771 |
| | (part 2) [tac pR, mCherry | | |
| | and LasI generator] | | |
| BBa_K180013 | Rock-paper-scissors - | . . . caccttcgggtgggcctttctgcgtttata | 772 |
| | Rock secondary plasmid | | |
| | [rhl pR, LacI generator] | | |
| BBa_K180014 | Rock-paper-scissors - | . . . caccttcggggggcctttctgcgtttata | 773 |
| | Paper primary plasmid | | |
| BBa_K180015 | Paper - Primary plasmid | . . . caccttcggggggcctttctgcgtttata | 774 |
| | (part 2) [tac pR, GFP and | | |
| | RhlI generator] | | |
| BBa_K180016 | Rock-paper-scissors - | . . . caccttcggggggcctttctgcgtttata | 775 |
| | Paper secondary plasmid | | |
| | [lux pR, LacI generator] | | |
| BBa_K180017 | Rock-paper-scissors - | . . . caccttcggggggcctttctgcgtttata | 776 |
| | Scissors primary plasmid | | |
| BBa_K180018 | Scissors - Primary | . . . caccttcgggtgggcctttctgcgtttata | 777 |
| | plasmid (part 1) [LasR | | |
| | generator] | | |
| BBa_K180019 | Scissors - Primary | . . . caccttcggggggcctttctgcgtttata | 778 |
| | plasmid (part 2) [tac pR, | | |
| | mBanana and LuxI | | |
| | generator] | | |
| BBa_K180020 | Rock-paper-scissors - | . . . caccttcgggtgggcctttctgcgtttata | 779 |
| | Scissors secondary | | |
| | plasmid [las pR, LacI | | |
| | generator] | | |
| BBa_K206000 | pBAD strong | . . . tgtttctccataccgtttttttgggctagc | 780 |
| BBa_K206001 | pBAD weak | . . . tgtttctccataccgtttttttgggctagc | 781 |
| BBa_K2558001 | lux pR-HS | . . . caagaaaatggtttgttactttcgaataaa | 782 |
| BBa_K259005 | AraC Rheostat Promoter | . . . ttttatcgcaactctctactgtttctccat | 783 |
| BBa_K259007 | AraC Promoter fused with | . . . gtttctccattactagagaaagaggggaca | 784 |
| | RBS | | |
| BBa_K266000 | PAI + LasR -> LuxI (AI) | . . . caccttcggggggcctttctgcgtttata | 785 |
| BBa_K266005 | PAI + LasR -> LasI & | . . . aataactctgatagtgctagtgtagatctc | 786 |
| | AI + LuxR --\| LasI | | |
| BBa_K266006 | PAI + LasR -> LasI + GFP | . . . caccttcgggtgggcctttctgcgtttata | 787 |
| | & AI + LuxR --\| LasI + GFP | | |
| BBa_K266007 | Complex QS -> LuxI & | . . . caccttcgggtgggcctttctgcgtttata | 788 |
| | LasI circuit | | |
| BBa_K3205003 | luxPR_3A | . . . caagaaaatggtttgttatagtcgaataaa | 789 |
| BBa_K3205004 | luxPR_3G | . . . caagaaaatggtttgttatagtcgaataaa | 790 |
| BBa_K3205005 | luxPR_4G12T | . . . caagaaaatggtttgttatagtcgaataaa | 791 |
| BBa_K3254014 | Optimized Ptac promoter | . . . taatgtgtggaattgtgagcgctcacaatt | 792 |
| BBa_K3254015 | Mutant Ptac promoter | . . . tactgtgtggaattgtgagcgctcacaatt | 793 |
| | No.1 | | |
| BBa_K3254016 | Mutant Ptac promoter | . . . taatgtgtggaattgtgagcgctcacaatt | 794 |
| | No.2 | | |
| BBa_K3254017 | Mutant Ptac promoter | . . . tactgtgtggaattgtgagcgctcacaatt | 795 |
| | No.3 | | |
| BBa_K338029 | +OmpR, +(CinR-HSL) | . . . tgctttccacgaacttgaaaacgctggagg | 796 |
| | Double Promoter | | |
| BBa_K427003 | Pm promoter of Mu | . . . tcctcaatatcctgtgatgaataaccgtac | 797 |
| | bacteriophage | | |
| BBa_K427004 | Pmom promoter of Mu | . . . tttttaagatagtggcgaattgatgcaaag | 798 |
| | bacteriophage | | |
| BBa_K658006 | position 3 mutated | . . . caagaaaatggtttgttatagtcgaataaa | 799 |
| | promoter lux pR-3 (luxR | | |
| | & HSL regulated) | | |
| BBa_K658007 | position 5 mutated | . . . caagaaaatggtttgttatagtcgaataaa | 800 |
| | promoter lux pR-5 (luxR | | |
| | & HSL regulated) | | |
| BBa_K658008 | position 3&5 mutated | . . . caagaaaatggtttgttatagtcgaataaa | 801 |
| | promoter lux pR-3/5 | | |
| | (luxR & HSL regulated) | | |
| BBa_K731201 | Arabinose inducible araC- | . . . ctctactgtttctccatacccgtttttttg | 802 |
| | pBAD promoter | | |
| BBa_K808000 | araC-Pbad - Arabinose | . . . tgtttctccatacccgttttttgggctaac | 803 |
| | inducible regulatory | | |
| | promoter/repressor unit | | |
| BBa_K864400 | Ptac, trp & lac regulated | . . . aatgtgtggaattgtgagcggataacaatt | 804 |
| | promoter | | |
| BBa_R0062 | Promoter (luxR & HSL | . . . caagaaaatggtttgttatagtcgaataaa | 805 |
| | regulated -- lux pR) | | |
| BBa_R0065 | Promoter (lambda cI and | . . . gtgttgactattttacctctggcggtgata | 806 |
| | luxR regulated -- hybrid) | | |
| BBa_R0071 | Promoter (RhIR & C4- | . . . gttagctttcgaattggctaaaaagtgttc | 807 |
| | HSL regulated) | | |
| BBa_R0078 | Promoter (cinR and HSL | . . . ccattctgctttccacgaacttgaaaacgc | 808 |
| | regulated) | | |
| BBa_R0079 | Promoter (LasR & PAI | . . . ggccgcgggttctttttggtacacgaaagc | 809 |
| | regulated) | | |
| BBa_R0080 | Promoter (AraC | . . . ttttatcgcaactctctactgtttctccat | 810 |
| | regulated) | | |
| BBa_R0082 | Promoter (OmpR, | . . . attattctgcatttttggggagaatggact | 811 |
| | positive) | | |
| BBa_R0083 | Promoter (OmpR, | . . . attattctgcatttttggggagaatggact | 812 |
| | positive) | | |
| BBa_R0084 | Promoter (OmpR, | . . . aacgttagtttgaatggaaagatgcctgca | 813 |
| | positive) | | |
| BBa_R1062 | Promoter, Standard (luxR | . . . aagaaaatggtttgttgatactcgaataaa | 814 |
| | and HSL regulated -- lux | | |
| | pR) | | |
| BBa_K1614000 | T7 bacteriophage | TAATACGACTCACTATAG | 819 |
| | Promoter | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of creating or modifying a cell-free expression vector, the method comprising:
(a) obtaining a cell-free expression vector comprising
(i) an origin of replication (ori),
(ii) a nucleic acid sequence encoding a protein or RNA,
(iii) a promoter arranged to drive expression of the protein or RNA, and
(iv) one or more selectable markers; and
(b) inserting into the cell-free expression vector an insulating terminator sequence at a location that is between 0 and 10,000 nucleotides in a 5′ direction from the promoter.

2. A method of performing protein or RNA synthesis in vitro, the method comprising synthesizing protein or RNA in vitro using a cell-free expression vector, wherein the cell-free expression vector comprises:
(i) an origin of replication (ori);
(ii) a nucleic acid encoding protein or RNA to be synthesized;
(iii) a promoter arranged to drive expression of the protein or RNA;
(iv) an insulating terminator sequence located between 0 and 10,000 nucleotides in a 5′ direction from the promoter; and
(iv) one or more selectable markers.

3. The method of claim 1 or claim 2, wherein the method avoids or reduces read-through of toxic product proteins during plasmid generation, while avoiding or decreasing a reduction in protein synthesis of the protein during cell-free protein synthesis.

4. The method of any one of claims 1-3, wherein the method avoids or reduces production of toxic RNA products.

5. The method of any one of claims 1-4, wherein the cell-free expression vector with the insulating terminator enables synthesis of the protein or RNA at a higher yield, with a higher growth rate, and/or with fewer sequence mutations than synthesis of the protein or RNA using a cell-free expression vector without an insulating terminator.

6. The method of any one of claims 1-4, wherein the cell-free expression vector with the insulating terminator enables synthesis of the protein or RNA at a higher level compared to a level of synthesis of the protein or RNA using a cell-free expression vector without an insulating terminator.

7. The method of any one of claims 1-6, wherein the cell-free expression vector contains a gene for a protein or RNA that inhibits or slows cellular replication in cells containing the cell-free expression vector.

8. The method of any one of claims 1-2 wherein the cell-free expression vector contains a gene for a protein or RNA that reduces plasmid yield from the cells containing the cell-free expression vector.

9. The method of any one of claims 1-8, wherein the insulating terminator sequence is rnpB-T1 (SEQ ID NO: 820), rrnB T1 (SEQ ID NO: 14), L3S2P21 (SEQ ID NO: 318), or L3S2P56 (SEQ ID NO: 319).

10. The method of claim 9, wherein the insulating terminator sequence is rnpB-T1 (SEQ ID NO: 820).

11. The method of any one of claims 1-10, wherein the promoter is a T7 phage promoter, a lac promoter, a trp promoter, a recA promoter, a ribosomal RNA promoter, a Sp6 promoter, a araBad promoter, a pTac promoter, or a J23119 promoter.

12. The method of claim 11, wherein the promoter is a T7 phage promoter.

13. The method of any one of claims 1-12, wherein the insulating terminator sequence is located 27 to 37 nucleotides in the 5′ direction from the promoter.

14. The method of any one of claims 1-13, wherein the insulating terminator sequence is located 35 to 37 nucleotides in the 5′ direction from the promoter.

15. The method of any one of claims 1-14, wherein the insulating terminator sequence is located 37 nucleotides in the 5′ direction from the promoter.

16. The method any one of claims 1-15, wherein the insulating terminator sequence is 0 to 10,000 nucleotides in a 3′ direction of the ori.

17. The method of claim 16, wherein the insulating terminator sequence is located 30 to 40 nucleotides in the 3′ direction of the ori.

18. The method of claim 17, wherein the insulating terminator sequence is located 40 nucleotides in the 3′ direction of the ori.

19. The method of claim 2, wherein the synthesizing step comprises using a cell-free protein synthesis platform.

20. The method of claim 19, wherein the cell-free protein synthesis platform comprises a system for in vitro transcription of mRNA and/or translation of polypeptides.

21. The method of any one of claims 1-20, wherein the cell-free expression vector further comprises a Ribosome-binding site (RBS).

22. The method of any one of claims 1-21, wherein the cell-free expression vector further comprises an Open Reading Frame (ORF).

23. A kit for use in a method of creating or modifying a cell-free expression vector, the kit comprising:
(a) a cell-free expression vector comprising
(i) an origin of replication (ori),
(ii) a nucleic acid sequence encoding a protein or RNA,
(iii) a promoter arranged to drive expression of the protein or RNA, and
(iv) one or more selectable markers;
(b) an insulating terminator sequence that is to be inserted at a location between 0 and 10,000 nucleotides in a 5′ direction from the promoter in the cell-free expression vector; and
(c) one or more cloning reagents.

24. A kit for performing protein or RNA synthesis in vitro, the kit comprising:
(a) reagents for cell-free protein or RNA synthesis; and
(b) a cell-free expression vector comprising
(i) an origin of replication (ori);
(ii) a nucleic acid encoding protein or RNA to be synthesized;
(iii) a promoter arranged to drive expression of the protein or RNA;
(iv) an insulating terminator sequence that is located between 0 and 10,000 nucleotides in a 5′ direction from the promoter; and
(iv) one or more selectable markers.

25. The kit of any one of claims 23-24, wherein the insulating terminator sequence is mpB-T1 (SEQ ID NO: 820), rrnB T1 (SEQ ID NO: 14), L3S2P21 (SEQ ID NO: 318), or L3S2P56 (SEQ ID NO: 319).

26. The kit of claim 25, wherein the insulating terminator sequence is mpB-T1 (SEQ ID NO: 820).

27. The kit of any one of claims 23-26, wherein the promoter is a T7 phage promoter, a lac promoter, a trp promoter, a recA promoter, a ribosomal RNA promoter, a Sp6 promoter, a araBad promoter, a pTac promoter, or a J23119 promoter.

28. The kit of claim 27, wherein the promoter is a T7 phage promoter.

29. The kit of any one of claims 23-28, wherein the insulating terminator is located between 0 and 10,000 nucleotides in a 5′ direction from the promoter

30. The kit of any one of claims 23-29, wherein the insulating terminator sequence is located 27 to 37 nucleotides in the 5′ direction from the promoter.

31. The kit of any one of claims 23-30, wherein the insulating terminator sequence is located 35 to 37 nucleotides in the 5′ direction from the promoter.

32. The kit of any one of claims 23-31, wherein the insulating terminator sequence is located 37 nucleotides in the 5′ direction from the promoter.

33. The kit of any one of claims 23-32, wherein the insulating terminator sequence is 0 to 10,000 nucleotides in a 3′ direction of the ori.

34. The kit of any one of claims 23-33, wherein the insulating terminator sequence is located 30 to 40 nucleotides in the 3′ direction of the ori.

35. The kit of any one of claims 23-34, wherein the insulating terminator sequence is located 40 nucleotides in the 3′ direction of the ori.

36. The kit of any one of claims 23-35, wherein the cell-free expression vector further comprises a Ribosome-binding site (RBS).

37. The kit of any one of claims 23-36, wherein the cell-free expression vector further comprises an Open Reading Frame (ORF).

38. A cell-free expression vector comprising:
(i) an origin of replication (ori),
(ii) a nucleic acid sequence encoding a protein or RNA,
(iii) a promoter arranged to drive expression of the protein or RNA,
(iv) one or more selectable markers, and
(v) an insulating terminator sequence at a location that is between 0 and 10,000 nucleotides in a 5′ direction from the promoter.

39. The cell-free expression vector of claim 38, wherein the insulating terminator sequence is mnpB-T1 (SEQ ID NO: 820), rrnB T1 (SEQ ID NO: 14), L3S2P21 (SEQ ID NO: 318), or L3S2P56 (SEQ ID NO: 319).

40. The cell-free expression vector of claim 39, wherein the insulating terminator sequence is rnpB-T1 (SEQ ID NO: 820).

41. The cell-free expression vector of any one of claims 38-40, wherein the promoter is a T7 phage promoter, a lac promoter, a trp promoter, a recA promoter, a ribosomal RNA promoter, a Sp6 promoter, a araBad promoter, a pTac promoter, or a J23119 promoter.

42. The cell-free expression vector of 41, wherein the promoter is a T7 phage promoter.

43. The cell-free expression vector of claim 43, wherein the vector comprises a backbone.

44. The cell-free expression vector of any one of claims 38-42, wherein the backbone is from one of the following vectors: pJL1, pY71, p70a, pBEST, pEXP5, or pT7CFE.

45. The cell-free expression vector of claim 44, wherein the backbone is from a pJL1 plasmid.

46. The cell-free expression of any one of claims 38-45, wherein at least a portion of the cell-free expression vector comprises the following sequence: gggcggagcctatggaaaaacgccagcaacgcggcctttttacggttcctggccttttccggcttatcggtcagtttcacctgattt acgtaaaaacccgcttcggcgggtttttgcttttggaggggcagaaagatgaatgactgtccacgacgctatacccaaaagaaa gctggccttttgctcacatgttcttatcccgcgaaattaatacgactcactatag (SEQ ID NO: 818).

47. The cell-free expression vector of any one of claims 38-46, comprising, consisting of, or consisting essentially of the features shown in FIG. 5.

48. The cell-free expression vector of any one of claims 38-47, wherein the insulating terminator is located between 0 and 10,000 nucleotides in a 5′ direction from the promoter

49. The cell-free expression vector of any one of claims 38-48, wherein the insulating terminator sequence is located 27 to 37 nucleotides in the 5′ direction from the promoter.

50. The cell-free expression vector of any one of claims 38-49, wherein the insulating terminator sequence is located 35 to 37 nucleotides in the 5′ direction from the promoter.

51. The cell-free expression vector of any one of claims 38-50, wherein the insulating terminator sequence is located 37 nucleotides in the 5′ direction from the promoter.

52. The cell-free expression vector of any one of claims 38-51, wherein the insulating terminator sequence is 0 to 10,000 nucleotides in a 3′ direction of the ori.

53. The cell-free expression vector of any one of claims 38-52, wherein the insulating terminator sequence is located 30 to 40 nucleotides in the 3′ direction of the ori.

54. The cell-free expression vector of any one of claims 38-53, wherein the insulating terminator sequence is located 40 nucleotides in the 3′ direction of the ori.

55. The cell-free expression vector of any one of claims 38-54, wherein the cell-free expression vector further comprises a Ribosome-binding site (RBS).

56. The cell-free expression vector of any one of claims 38-55, wherein the cell-free expression vector further comprises an Open Reading Frame (ORF).
